SEQUENCE LISTING (276 sequences)

SEQ ID NO: 1 - DNA, 46208 bp, Homo sapiens
  source 1..46208: mol_type = genomic DNA
actcctcctctctgcccctcagctcgctcatctttcttcccgccccctctcttttccttctttggttctttgaagtgatgagctagcgcaaccacaaaccatacattccttttgtagaaaaacccgtgcctcgaatgaggcgagactcagagaggacccaggcgcggggcggacccctccaattccttcctcgcgcccccgaaagagcggcgcaccagcagccgaactgccggcgcccaggctccctggtccggccgggatgcggccggtacccgctccccgccgggaacaacctctccactcttcctgcagggagctggtgccagccgacagccgcgccagggccgctccgggtaccagggtcggatcgggtgacgtcgcgaacttgcgcctggccgccaagccggcctccaggctgaagaaggacccgccccggccttgacccgggccccgcccctccagccggggcaccgagccccggccctagctgctcgcccctactcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggtaagacccgagcccgcgcccgggatagtagtcccggactgagagaggagccgggctggaaaagcagcggatggcagagtgtggcaagcctaggtccagggcgcggagagatgtggggataaagtgagactccggccagtgtgccagctggatgcatagaagtcccttggacgattcgtcttaaacggacgttattgcgcccccactatgagatgggagtatttagaggagcaggacccagtcctgcctgcgaggggcttaaggtggggtgcgtagaagcactgacagttatccagagctagggactgaatcggatccctcacgcagctctcatttactgctagatctttcaaagaatgctttatctcaatctagatctttcgaaaaatgctttatctcaatcttattgaaagggaaaccgcgtcgaagaagctgaataaattaacaggcaccatccccgcaaaaagggagacgtgataagaatggacaccagatctctttgaccccggagcttgagagtaattaagctcacagctttggggtagattctctgttcaattaccttctatgtgaccttgagcaagttatttactctttcttgcctcagttctatcctgtataaaaataaggattataattgtacatagtgcacatagtgtggtctctatattattaccagacacatagtgtggtgtctatatatgttgttattaccagacactataatatagggttaagaaaaggaaagaaagaaagaaagagaaagaaagaaagaaacttcagagagagccaaaatgtggaaagacttgggtttcaaaggatttcaataaagtaggtttggcggcagagatttaggaagagtccaacttaagagataatgggctcttaaaacatgtgcaaaagtttagttcaaagtcccaaatgttaggttaaaggtaatgccttttctattgcactaaattctagaaagggagcagaggtggttgcacagtaataaataagtacaaccaagatgtatatatggtggttaaaaaaaaaataaaaccaccacccaaatttaaccctgtgtaaaagccttgagtaaagggattcagcaagactgtggagaaacttgggagaagaaaaaagagctttggaagaggaagaactgagaaatctccagagaaacgtccagagaaatctgtgaagtccacaagtgtggaagggaaagggaagtgtgattaggctgggatacagaaggctaacagtacactttatatgagaagagcttagaggtcaggagagcacaatgctgaggggtttgtttggttttattttcccctctggaaacttggagaaaggaggacaggaaaagatgataagagggcaccattgtttggttttaaaggtccctccccaaccccccacaacaagaaagctcattttaatgagatgagagaaggggagaaagctccctccgttctgatgcttttgagcttatcagggacaccctgcagcagaaattccagctgatccttcagccttcagagttgatgaggtggggctcacacacatctggatttgaagaagaatcgtgggtttcaagaactctgtggtctcttaagcattggtattccacagctacacttccctgatactttgcactcagtaatgacaaaataaatatttgtagaattgagttgttgctaccctgtgtctccacttcctcccagaagggtagaataaacttacatgtgtttgttttattttgcatataaaccacgactttgtaaactttactttttgtgacactcttttctccacaattgtgtgaaccctttttagtggaaaaataagaatgaagaattcctagttttccctaataatatttccctcttgtaagtctagttgagctcctggtgttaatcatggtatcaaatgtttatgacaggacatctaggtgaggcctgagtaaactgctgtaagttacagcctatctcttctgtgtttgttcagaatcctttttgttggatgaaattactttctttcaaattttgctcaagaaaattgaattgggttctgaggaaaaataggctacttacgtaatgttggcagggggtaaagattgggagcggatcacatggcaaggttagatggctgtgtcctcaggacttactctgtgcagtttgggggtgtgtcaaacatgatttgacgtgcataaaatcccagttctgtagtgctaaagcatgtgctaaagctctgctgtactccagtattgcacagatgtttcactgtcaattgtttatattatactaaccctcagagaatttattgctgttgtggtataaacgttcctccactgtaaccactcaccgcatattgattcctaggacactggggattcaaatcagggtggaaaacagttgtggaggggcatctccagtttcctggagactggacctagttcaaaggagccacataaaaggactgaagcatatggaggacaaagaggaatttcctagaatccagctaaaacacaggagtcccctcgagcaggaacttccaaggaggtgtacactttctagacctactggagtaatcctgattgctgtagaggacccagggcaatcctggagaccccctggaaccactactaggcagaaaactgggaagaggatggcaaaactctccagttagtgtagctgtctcagtggaaggactgttgtcccctgaggcacgaggaagacagcatggaagcactgacagtatgcgcttggcctggttactttctctgagcttcatgttccttagctgtaaaacaggagtgataacccccatcttgcagaggttttgtgaggaatacatgagataatccctgctggtgctcactgaaagtgagtttcatgtgccctgggtggggaggaggggaattgtgaggaagggaaggtttgggttgtgcatcagattatctgattctcacagcctggcacagtctaccacctgcctgccctctaccatcttacctaagtaccattgcctaaactatttttgctatagttactcattgagcagacttatgaaatctccccacagcacaaaactgaaggtatccacctccagaggtgagattctcaccaagacaggctgtggggaaggtagctttgagcaggtctctagggtcccctagaaaggtgccttttctctgcttggctgctgcttctgaggccctggctgctgagcccacttcctgcccataaggctgcagccttagccctagccaattgctcctaaatgggggaggcagcagccagggtgggaaccatggcccccgcctttaactcacccaggggacaggttagtcagatagatggtcctttgttcgccaccctccacctcccacatagacaacattctaatgtgtttgatggactgcatatagttttgtttctacatgcactttcaaaacctttgtgtgcttatattttataatggcatacccagtcctgttatttagccctttctgtttcttacttttgtcctgaacttattgtttttatgatttgtccatgttgccctagatgcccttatacctatacatcactgtgaattgtgctctgtgtattttacccatctccacgtattttacccatctgctccctggctgatgggtttggagatggcgtctaacaccctgcctccacaaataatgctgctccagatatcttcctggatggtctcaccagatgatttctacccaggagcagaattgctggccacagggcatactttgagtactacaagactggtttcctcaatgactgacatcctccattctcttccacctactcccatccccaaacaggacatgtaaatttctatattttacctatatatcatggcagtatccagctttattgtaggtgtaaaatacacattgctgctttatcatgtacttttctaataactagtgagtttggacattttttgatatgtttgttaactttttaagcttcctcttctctaattgcttattcatattctttggtattttttaaattgggttagctttatattttttgactttcaggagttataggttttgatctgtgatcaattttagacattgcaaatatattatgatgatattaggtctattaattttgtccatggtatctttcattgaacagaaattcttcatttcttgtaattgaattaatctttttttccttgtggcttgtgcttttgaaagtttgcctaatagtttgctaatttaaagcttatcacctttaacactcctcactggaaattcagagatcaccacggttctctctgccttttaagagataaatctacaatctgtggctatgcaattcacttctcagcatcaccagttcctcatctataaaatagaagtttgaaccagcttacctctgaggtcccatacatatttgaacaactggagccctacagtgaaatgttttgattcagtgaagggacaggaggatggggcaaaggtgtagaggcaggaatctacacaatggaccaggttggaccagagtgaggtagcaggaatcatggcagaagaggttgtgtaaactctaaaggccacctgagaagtttgagttatgttgccatgggcagccatgtggtttctgcgtagaagaccactgtggtataataaaaaataaatatttggtccttgtccccagtacctgtcacagagttcctaaaaccctagaaatttcctaagtaatagaaatgtcctttgtgattcataataaaccccatgataatacctgggtttatgctcctgcagacttagggtgagatccctagatagcttcaagatggaaccagtcaccagcaagtccaagagattaggggatcaaagagttggaactttcggtaccacccaccaaagtccaggaaaggagggtgggactggagattcagctctacaaaaactcttgaacaagatgtgaggagctttttagttgctgagcatttggaggttcctggagggtgcacacccaggtagggcatgaaagctccgcattgcccccatatcttggcctatccattcttcacttggctgttcatctatatcctttgtatagcctttataataaaccaatgagtgagttctgtaagctgtcctagcaagttaattgaagccaaggaggaagtaatggaaacccctgatttatagccattcagccagaagcacaacctcaggcttgtgatcagcctgaagtcagggcagtcttgtaggattgagcccttaacctatgggatctgacaatgtcttgagatagtatcagaattgaattcaattataggacactcagtttgtgttttctggagtactgtttggtgtctgggggaaatcccacacacatctggtcacagaagtattctgtgttcagtgtgacagtagaaaaagcagtttgtgtgtatgtgtgtgtttgtgcttttgtatgctaatgagatgatctatggctgggggcccttagatagcttcgggaagggggctggtcgccagaaagactaatttatgactagagggttagaactttcagccctacctctcccacttctagagggtagatgggctacagattgagttcggtctccaacagccagtgaattaatcaatcatgccttcacgataaaccttcatgaagacccctaaacaaccagattcagagagcttccaagttggtgaacatattgaggtgctgggaaggtggtgcacatggagtgggcatggaagctttgcccatccactcccatgccttgccctgtgcatctcttcacttgcttgttccctagtcgtatgctttataataagccagtaaagtgctttccaagttctgtgactcgttctagccaattatcaatcccaaggatagggtcatgggaacctccaatttataactagttggttgggaatacaggtggcccaggactctcgactggtgtctgaagtgggggcagtcttgtgcatttgagccttctcacttgtgggatctgatgctaagtccaggtagagaatgtcagaatgcatctgaattgttggacaccaagttggtgtccacacagaattggggaattggttgatgtgaggaaaaaccctcccagctacatagggaaatcacggttttggaagatgagcagaaaagggtgggagggagaagacaagagagaagagggtgaggaaagaggccaggcagcagtccaagtgtgagatgataacacctgaactctggtaattgaggagaccaaatccacgggaaccagctgcaaaaggcattctgtctggtaggtatagggctaagggagtaaacgtgggggtgagactgggatcaagtccccttcctaccacttagaatctgtggactgggagcaggttatttaatctcatgaaacctccatttccacatgtgtgaggtatgtgattgttgtctccacccctagggtgctgtggggtttacaagagatagtagtgcacataatgtgcttagcacagggccagcccctagtaagtgccggctgtacttgccttctctttgcaggggtggctcaggagagggtgtaatcaaaagatgattatgagggttcaagcaccaggcaatgggagagtcagcaggttttaatgcttccacacagaggctggattcttctacttccgcttaccgctgacagctgaggcctgctacctcaagccctgaaatgcagtctgaagacttttcctggacttactgggcaccattggactgtacagggctgtggcagacaggagcagtggaaagagatttggatgggacgtcagaacacccagattttacttcttggctcttccacttactagctgtgtgaatttggcagtggtttccttatctataaaatgggggaattggagagggtggtctactgcatgttacttctcagcacctcagctcctccccacccctctctcttgcttctaatctgcctacatcacttggtttgtctctggacaagtctaagtttgcatgacaactctctaatttctccttcacagttatttatttattttttggcagctatttccttgtggagagatagcgtaggtctttcaggtgcttccatttggccactgtttgcaggtgaagaggtttccaggatgctcgcgggatgttactctggtagtaacagaaatgatggcctggggaatgggaagagtatgagcccagttttgagcctgagtctttggggctcatttattcattcaatctgcaaatcttcattcagcattactacatgccaggctctctgctaagtgctgaggatagcgatcaggacctgctagggtgaaaattactcttcagaagatagcagagtggataatggccattcgagttcctcttaactacacagagggactgggtaagatctataaaggagagaggttaaacagcaaagcaaaagtgactgaaattctaacacccatgcatggggattcttgagctaaagatctggtctgtagaagaagacacatcagaccctcatatttagcatctaagccgaaatcagcagtaagcatgtttcttctcccataaaaacttgtccctaagaagagagacaagtgcttacaaggactcttatgttaaaggaaactgtcacaattactttatcttcagtcatgaagcctttcttgaggaaagaaaaattagcaatactagtttgggaagcttgaaagagagattctgtttgcaatgacacttttaaaaaggcaacaacaaccaaaaaagtaattattcctaaagctcaggtgtcagaagttgtgaatgtctagggggttgtggatccccaacctcagagacaggaacagcaccactcccatcctggggctgtggcctcaggcctctgcttcctctttttcagtctaaattccagagcagagaggtgctggatgaccagcttcaggcaggggagggaggaagtaaaggtagtttctcagctcatcttgtcctcgtactattaatagcatctgttgttttcttaactgaaaattggagagaaccaaacaaatcaacatcatattggcggctttctgcacatcactcacaaatacatctgaatgctctgtactctccagtgtttattttcctcagccacataatcggaatttgaacattcttttgacacttcttttccctcacttgaataattagttctatatataatccaaatattagctggagccctctagcccctcagagatatagttgaaccttcctggacctcaattagttgggcagttaattctgaatgttgatgtgcctattttgcacgtatggagagtttgtacgtcccccctccccaaacaagtagaatgctgtctatttggtacttgctcattcatacattctttcaacaaccaaacccagttgcagctgggttgaaccccagggtgtttcctgctattcagtgtgatgccacattcacctgctgtggccaacattccttggcccaggactgcagggatggaagccaacccaacatcagaagtgggcaacaatgccagaaacctatcaaccaaaacaaatgtaatttgtaatcctccctggatgatgttgagtgattggcaactgagccccgtcatatagtaaatcaggctgtctctctttaaaaaagagagacagagagagagagagtctcccaatgttgcctaggctggtctcaaactcctgggctcaagcaatcatcccttcttgcctcccaaaatgctgggattacaggcatgagccaccatgcccagcctaggctgtctctttagctcttcaattatagggcttcctgcaacctcagcccttaacgaatgagctagcgtgtaagtgactagctaaataaatgtgctgcaaacctctgacaaatggctttttgacattattaaaatttatttatgtggaacaaaaaaattcttttcttctgcatcatgcatcctacactgcttttaaagaaactctgctatcctttgttttaccgtctaacatttctttgtttcccttctgacatgtttcagcctcttgtcatcacttggaacatttttactgcctcatccctctagtttccatttcctagcctagcccagcccagtatttttgaaacccaaatacctggaattttgcctgatgtttaaagccaaaaccaaatgaaaatgtgaagaaccaaaggagattttattcttctgtgactcaaaatcctgttgttattgttaaatgaaccctgtgggatacagtctggtatttttaacaccttctgggggtatttgtcaatatttacatttgatgtggatagcttataaaagtccagtggctttgggtaaaccctgttcactaagaataagacccagaaggtcatatgaatcaacgatcaaagaatgaagatattctgaatccaagtttcttgttttgattattaaaaataccttgcaattagttaggcatgagagaaagcttgtgtataaagtttggcttgtaattcacatgaattctctatacaggccaaaaactaccttccctttgggaatatacaatgaccttatttggaaattacttcaggtaattatgaagtaataatatctctattcatatgctaaaaagaattcaaaataacattcaattatcatctcttagaaactaaattaaacgggtactcacccaagttgaaatgcaaacaaatgcatgatatcattatgtgaccctcccctagcccttaccctgtccatggcagatgctaagaaaaacttgtccctaagaagagagaagtcaattgatttagtcagcctttcccactgtgtttggactgagcctcagaaccctccttatccccatactgcaaggagttactgtcaatcaactagagatggcgctcaagataaaattattttccatcctgtactaatataataataagcatggaatctatttattggttttttcacaacaataaaatatatctttagcaagtaacaaatggtagaggtaatacttttagataaagagggaagaatccttctgacttttctctttatcacattttacccacacacaatgtggatggaatctgcaactgaatttagcagtcacactgaaactctgctgcatgaccacactgtcctgaggccatgtgccaacttctatgtttttttttcccaaagctcttccaaggaagagctgccacttctccatttactcttttcacctgtaaatgaatgctgccgttggtattgagaaaatgggtgaatgaaagaaatcatgagaacactcctggggtgagagcaagaggaatgaaaacacgtggtgtgagagcaggtcagagatggtggttaacacaggaaccaggatggaggcctcagacttgaccctggcctgcaggacgttggcctgcattgtctgcaggaggaaacacagaggtcagtggatatcctgcggtcttccattaaggcctcttactattctggaacattttatttttatagagaaatctcccacttcctgatctccccagttagaattgattgatcaaagcagtccctctccctgttattaaataattaaataatatgcattagttgatccctataaccacttaaaatgtatctaacacaggagaaatctttggtaaatatttactgaatgaatgaacaaataaataattggagggagggagaaagagacagagttaaatcctgaggctggagtttttggtccttcagccccttatcccattgcccctggcagccccatccatgtgaccttgaattctgtccaagtctcatcactttctatgtgtgttgtaatggaaaaaaggttacaaaacacagtctttctggcctggagtaccaaaatagtaaagacttctgttttgaaagcctctgacaaatgaacacaacagccacagtgataactttagcaggaaattgaaccacaaaaagcaagcgagccacaaagccctgaaggctaaaaatgcctcacaggccaagaggcagttaggttgcacctgagacagaggctgctttcttaactgggaaatgggtgtctccagatatataaagctgtgaggagatcaccatggcccagctctgacgagccactggtacagcattgttggttagtttgcctcaacctgcagcagagtttttattccaagagttaacagttagttaggctttttccaaaggttgagatgacaccccaactcccacacccatttccgtcctgccccagcttgaaaccaggcaaagtgagttaagacattgggagatgggaggggacaggaggggtagaggcctggcacccctctctcttctgtttcaagagacactttgtggagttgtctagagagactgggggtaactgctccttctgcccccaatctccccttagctccccactcctccctatcctgccaggccaaccaccagcagagaagggcgcttagctccagtgacgctgtgagtgaggtacttgtgaggatgcatatggttcctgttggaaatgcacggaggagcagtgaaacaccagaagccccatcctctctcctgcccgtgccctcatgcccagccagctgaatattcctgtggcttccactcctgcactgtcccctcagtgtcacctccccaccacattcccacagccccacttgctgtgtcacctctcccctgagggagaggtgtcttcctgccagtctcttcaccaccaggctcacacaactcccatccattcactgcaggctgccagactgagctttccaagcacccccggactgcctccagccctactttctcttcaaggcttcccacttccctcaaaatgaaagccctgactccttggacaccatacagaccctccctgatctcccagcgatgctcttagcagcatcatttttttttctgccagattttcaggtgccctcagttccaggagcaccaaatcacctggatgccatgagctgcttatgacacagttcaaggtgtgacatctttgaggtctctccataacacctctactgcagacgtctgtctctggtgtatcagctgtaccttgcacacatgtctctaaccatctgtccactgtgtctgtctgttcacacatcagattgaccctatgttctctgtgacaccaggtgagctctggcaccagtgcccagcagatgattactgaatgattaataaaactactcactccacctgctgcttttaaatgggctccattttggtatgatttggtgcgtgaaccattaaaacttttcagaagtcaggatttgaaacaaacttggtaaaaagtagttaagcaatgtcttcgttgtaaagtgagaaaccaagttgcttggaaacaggtgaaaagcttagccagcagcttcacaattcacagcctggcactccagtagccctctctggacaattgcccaggttttcttgttttcatttctgtgttttaatttacaagttacctttatatttctactaataccttactggcacttgtttgtgaatgaaacaatttttttgagagtttgttaattgttctgtgtgagtgtggaaagtttctttcatataagatcatcaattctttttttaaaaatagtgttttaggttttctcctacataaagaaagtggtgggggtgaggagctgggggtggaccagatgctcacattgtatttctatgattttgctgttggagatactgaagatcattttgctgggctttgctctcttaaattacagaaaagtgcatttggtgaagtttagtgtttttagtaaacatatacaagatgtcatagggatagaattagtgaaggaatagggttcgtgaatgaatgagctcaatcttagtaatgaaatgtttggaaagcaacagaacaagagagaaagagcatcagaacatgatttaggtaccccatacatttatacaaataaaaaaaagatctgatttagggtcatagaaatcacatacctaaactgatggttaaacaataagtggcaagtaacagtttagctacaaaaaatgtacaggaacacataaggaaagttacatgggaactttcagagaggaccaaaaaaggtggaataagattatctgctctgttatgatcccctgtatttctctgctctatgttcttctcttgggcagtcttttcctgagatgttttcatgttattaatcaaaaataatagaaacgaataaactagatcaatgcatttacaagattttttgagtgcctgcagtatgcctcggtgtggtgggaacttggtgtactgaggtgagtagcatgatcctgaagagcttaggaatcactgagttctcgtaagtgcaatagaagaaaaactggtagaaggaagtcaaatgaaaatacaagtttctaaagaaaataagtatttacaaaggaatttgtctgcattaatttctgtggaaaagttagctatgtatagaagaaagaaacttgttagtaattctttcattcattcatttattcattcaacagtgaatccctgccatggtagtactaactacttgcactgtggtagtgcaagggatacatttgctgtgcaagtagttaggaattttttttttttttttttttttttttagtgctttgtgtatgctaggctttatgcaaatatacagccaagcacagaggttaagaactttggaatcaaaccgaagtggttctagtcccagctcccccttacaaattgtgtgatcttcaacagttcatttaacttctctagatctcagattttctcatcagtaaaatgagtgcagtaagagtaccaccctcacagcatgaaatcagattttctgtactctttactaactcactttccatgacgctggcatgtggcaagtgctgactcaatactggctcatttccttaactgcttttattttattcattcactgccattccagaggaaactgcggttctgagatgtgccctgggtcactgctgaggagcggacggagcctgggagggagctacccctgccttgtttcctcacactttatggggtgggtgtcaggaatgaggctggaggggaaggcaaaaccagatccctaagagccccctggaccctgcttagggtgtttacttctatcataaaggctgtgcttctgtagagacctaaggcagagagtgccaaggtcaggtttgcatttgagaaaagtttttctggcagcaatataaagaatagtttagcaaaagaaatgagaatagctggaaacccagttaggaggctgtggtagtccaggagcacactaaggcagcaacaataggtggatggaaactggagcatttaggtaagggagagggatgccagaggacgtgatggtgagggagagggaagagggtgggactccaccgcaccagggcttctggctttattgactgggtatatggtggcatcctgggccaagacagtgagtcacagggaagaggctttgagcctggtaagcttgggacacttttgagacattgggaggaaatgacacatggcagttgacatacagatctagacgtcaggagaaaagtcagggctggagattttcggtttacagttgaaggtatgagtaggaatgagatcatttactcagtccatcccaccacattgagcaccatgaataaggggctcctctccccatggggcaagagaggtgaaaagacagtcaaagtcactactttcaggaggattaggctcaagttaggatggggaggtggagaaggcatcaaacaaataaatgaaaactataacttcagttattgccaagtgctaggaatataatgttgctgttaaactgcaaggtggctaacaagttggatagtctttcttgaagaactgtagaaacattcattaaaaacaaaataattttatagttaagcaagaaagtgattgtactataacaagggagacttatttcaccataggaaaaaaatcataacattctaagaatagaaggaaaaaataacaattacccaagcctgcaacaacctgtaagaccagatcagcttttaagaggaaagttaattagataacaggaagaggtaaaaatgagatcatcagagatgggggaaaaggcaaatttagaaattctgaatttaataggggagacaagtatgtaggaatataaacacattgtgctttctctgcacaaacacattcacatgaggatgtatacacaacccttgcaggcacaggtactcagtgaatgcctgcgtgccaaggagggggacagcactgagcaggagaggtggcagggcctggtggcccaggagcactcatatagtcaggagactgatataaaagaaagatcctgctaacatgccattacagagtatggaatgagcaaggaaggaagagggtgaggctgtaagagagtgcacaggccctcatttaaatggacgggtcaccagccagtacttcatgcctgttcctagatgacccacaaagtgaaacgaaactggatgtggtatttagcaagtctgagatttgctggtttaagtactaagttagcaaaccagccttggcagccagagcaggtagggtagggggcccaaggagatgggtgtatccaggaggccaatggaggaaggagaggaaggttttctgggaagccagctgacatgtcatgctttgtcagtggggcctctgcatggattccctccactcccccccagcccccaactcactacggggagctgcaaaatgacagacaatgggtggttttgtacccagcagccactcctcaaccagtgaggaatgggagagagataagaaggtgggaaacaaggattgcttgtcaccatccctgctcctcctgaagccctgcgctgggagcaggtacagaacccgctgaaggtcacctaatcatttcaccatggagttcttccccagatgtgaaactgctgggactggagcaaagggtgcccagagctgactggaggaggaagccagagccacactggaacatatggagatgctagctcctgcctgctattgaggaacttcattttcccagaagaattagaggaccacaggagtgcttgggaagaagcaattgtggcaacctaggtgaatgatgacctaggaggtgaaatgaggaaggaggcctttggtctctgccaaccatacatttcaggttgccttttgctaaagtcctgctgatgttaatcattctctccagcagatcgcggcttgaatcatggggtctgaaatgtgggtttatgagagaaaaacaacatggaaaaattgggcagaggctggtaaatgtgaacattcaaacctgcccccaatctgagatggtgggacagtggacccctggccagctctccaggtgttgcgtatgcacattggcaaaccacctttattaggtggtgtgacacctgggtagcaattctcaagcaatgtgatgatctgaatgtccctgggcctgacccaggtgaggaggacacaaaatcatgcaatatgtctcatagagcttagtcagttttgagaaatactggactaatgttatgtttttaaagcacctaattctttttctcttttctgttttatctaggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccaggtattttctacttttatagcactcaatgcttgaaaatagatgccctaaatcacagcttggcattatatttatggactgtagataaaagtaggaacaactgggtaaatagtgcactagaataacactgataaaagcaatctggtaaattctatgctgttgttccaaggtacttcttaaagagatgtcctcttctgctcagtgaacttctttgttgacaaaaacagtatctttgtctaattcagtgctttcaaaatgggaagaggagacaatgtatagccagtgctaaacagtacagtttcatttactagagaatgagagcctcaaaactcactgcccttctcctgtggagccatgagcacagagccagagttttacctcaatttaccttattgctccttttctaggatgattattacatgtccagctaactcacctttctcgcttttcattattcatctcgttttaatgacattcttgtatctgactttctgggaaatatatatagtggtcttatgatatatatggatatatataagtgtatgtatatacatttatagacatatatacacatatgtatatataatatatataatcttaagacacacacatacatatatgacacatatacatacatacatataagacacacacacatatataatcttaagacccttcaagaaactattttgaggcacttttataagaaaactttttggaatattttggcaatgagaacaaaaatgacattttctttaaattatccaaattaaatgtgtttccatatttgaagaaaaaaagggaaaataggagccagggttatctagaaacatgagttttcattaagaactaactgataaaggacatataattgacaattcaaaaatatatatttactgttgttgttgttataaaataaataagtccaggccattggaaacaacaacagaaaagtcagctgtgtttgctgagagcagcaagcgggtcctgcagctggcatagaggcagagctggaggcagggtggagcggctggggctggagtggtcagtgagaagagtgagagaacttgctaaagaaccccccaaccccaccctggggtcaggtttacttgctgaagaacctccttggtccccttgagagctgtgtaaaacatccctggggcacaggacattccagaagtgaggtcagagtcacaagctaggatgcaggctccatgaggacagaaacctctgctgtctcactattgcccaatgactgtgcacagtgcctggcatatgataagtcctcaggaagcattgctgggatgaatacatgagagcaggaaggtataggggccacagtcacagaacagcagatagttccatttcctggagcatgggttctgactaggaaaagctagaaagttggatcagggctagattgtagggctgagaagttcaggtgtgatttggaggaaagcattgtctcttctttaaaaggcacccataaggaggtaggaggtgcaggtcttggtggaaaggcacagcagctccagaaatcaaactgccccagctgggactctgaccagccctttaccaggctgtaacagagaggggatcaaaagtgacactgctgtttccagcagctgctggaagagagggctgcattttgcagggcccagtgtggggggaaagggatggggtggaatagaatgtttccaggagcactgcttcttccaagactacaggcccagcggcctggtgtgggttcttcctacagttctttgaccctaatcaagcccagttctcccagtcctagtggcctgggcacccacagggttataacctgtatttctagcacatttaatctaatttattttagctagatcattaaatacctgaaatacaactctcctcatgacatcttgaaatataagccatgctttgtatatatgggatgcctggtcctgtgaggtttctgatataatagaggaatacattcctttgtcctcagaggcatttctctgaattttttttccttcttggcagccataccttaagcaaaagatggtagcatactctcgacatttagtcattcccctgggtacctgtctgtgtgccaggcactgttttaggcaccagggatactgaccaaaaaaaaaaaagttgcccactggagcttacatattcagtgggggaatcaataaatgagtaaatggtttggtatgtcagaaggtgctaaggctgtggagaagaattaagcaggaggggaatggcaagtgcagcaggggggaaggtataccttgttgtttcaaagagatgctcaaggaaaggctcacatagaaggcaacatagcactgagggtcaacggggagccctgggcttcttggggcaaccaacaggaagcagattgtccttgatgttctcataatagacaacagtggcaaaactacaactcttgatttttcaacattgcctatgagttacagacagccttgcctgtccaacacatctgtgagctgctaattattaggttgtcattctggggcctccctggctatgatgtcagccatgaattatcttccatggtaatttgcacttgtggagttggctaatgttaaggattcaggagttcccctgctgaagcacacagacattcctaaacctcacagtggaacaagggatgcatttctaataatcttgttactctttggattagtacttttcaaactggagtttgcagactcctgcattttgaaagcttttaatgaaatgtcattctcccaccctccttcccttgtttagtgttagttcttagaaatcagtgtaagaacgtctggatctactggaggactccctagtcacagagtaccagacatgacttcagggcagcatgtctgtatttgcagtcatcttcatgccagtgatagccataaaacctcaaagcttcctcaaatgtggctaacagagccagttggaggaaccagttgccttgctgcacacagagaacctgggttgcacagtgagagccatgcttgactactgataacaaaaggctatcgtttgttaccactatgtgcagtaataagcacccagggaagaatgtacacaatcccagtgccagcaacatttagatgaggccctcatgattaacacagagctcaataatgctctaatgctggcattatttatatatttatatattttttgaggcagagtcttgctcttgttgcccaggctggagtgcagtggtatggtctcggctcattgcagcctccgcctggcaggttcaagacattctcctgcctcagccttccaagtacctggcatgtgcctccacgcctgaataatttttgtatttttagtagagacagggtttcgccattttgcctgggctggtctcaaactcctggcatcaagtgatctgcctgctgcagcctcccaaagtactgagattacaagcgtgagccactgtgcctggcctgctggcattaaagagaacatgacaaagttatcaactgccatattaaaataggcattcaaggactacttgctgacagttttaagtttgaacactgattatcccaagtttccaacgaacgtcttggagttgctaatactcttttgtgtatcatgcttacgtgcttaggcattttctgcattggtattgtggaagttgagatgtacaaatatataaaacaatatctgaagcctcctcacagtttgcaaccaagttcaagcaaaacaatatccacaaccttaaactaatacaacataaagtgaatgtaatgaggatattgatttgaatttttatttacttcttaggggttttgtatttaatgaataaatatgttttggttttactgactcttgagctttagtaaattaaggtgtttaacctttgcatgttaatatgtatattaagtaatataataaaaattgttaataaatatccattttatttattttgttccttagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggtaattgtttcaaaaggattgcatgggccaggatgtccagataagcactgtgtctcttttgcctttgtaactgttattactctttttactgctatttaatatgtaatgtatattatatgatctataatatatatgtaatatacattaaatgggaacatgtgcaaatcttacaaacattactaagaatttattgatttcatgattataagtcaattccatatatttatgtatataataaatatataagacataaaatatgtataaaacatatatataccatacatatgaaaaacaaaaagactattgagccccaaccaagtcatttatgatagattcttatcaaaaagcaacatgtcatataaaaactttaagcataaacaaaataatgtaaataaggacctttataactcaaggtggatttgatttgtaggttgcacaggtagcagaggctggacctcgagtgtgagtcctaactctgctgcagactaggaatataagtatcctggaagccacatgaccctgggtcctcaggagagcatgcactcttcctacccactacctgactacccccaccactctctttttttctctctcaaattggtgttaagtagttttcatcagaataactcttttatgatttttccactttaaacctataatgtcccaaaacttttgaatctggttccacctttcccacttcccccaccttgcactgcatagatctttgaagtattcccaaggaaaggaagaagaggctggtaaacagttcttctatcagcattcgcaaagaaggctttggcgtgggaacagccctccccttgaagaggctcctgcttctaccatccttatgtgggctgtcaaagaccatgctattttgataaaagcgaaactcaaaataccccaaacaataagtaggaaatacccaaaaacattaaaagggattatcactaagaaagtaatttttattttctttctagtactttggagtattttcaaataatgaatgcatgttacttttgcaatcggaaagcaaaatataaattataaaaaggaaagaaacacaatattgggtcaaaagtccccattcctaattcacttgaatgactttgagcaggtcacttagcacctctgaatctgctagcttagcttcaaaataggggatgatcatgctcaaagttccacgttactgtgaaaattatatgagatgtaaaaatatgtgaaagcagtttataaactataaatagctatattctggtatgcagcgtcatggaggttgcctgcttataaaaatatgtcagtcaaaatcataactcacaaactaaagttggccttcacttaagaaaataccagtatgaatgtgtgctaattaaattccttcacagatattttacttcttggtaaacacaggctaaggaataatttcaaactgagttaaactgacaggttgtgaaatcaatttatgggttatgaccagtagtagggcttttttttttgacagataagactaaaatagattaggcacttacattactcataagaatgataaatattgttccatgggaaaagatgtatataagatgtcaagaacagccccaggcacatagcagacaatccataaatgtgatgaggatgacgactagcactggacattgttagcatggataacattgcaatcaaagagtttcaacaactccatgagggttaaatgaactaagagagccaagacgaggaaataattggagcaggtcactgggaagcactgggtaggggtgaagactcaacaaagtccttctgactctaccactaatggtggggtcctttcaatgcctgattaccctcatctgtggaatgaaggtgataacaacagtacctattataaggggtcactgtgatttaatatgtgtaatgaatgtataggtaaatggccttagcacagggcctagtatgttagctgttcttattttagttatgtgctggctcactggataagacctgtgaaaacccaaaccttagcttttcactgaggcagtgtcagcacccttggacacacagtgggagctagatatgtctcctcccccttcttcctttagaataatggtttcaaaaagtttaaactatgacccgtagtaagaagaacattttagacagggagccaaaacacacaaacatatatgtgtgtgtgcgttcacatacgaacatgcaaaagcacttgtatgtgcaaacacatacaagtgaaagaaaactttcacaaagcacatggaatgcaccccaacttttctgttttactgttttattcacttaatgccagctaccactcattgaattgattttcttgtactgtagttaatccatgtttctaaaaactcagctttaaatgggacgtgaccaaaataatgcatgaacactcaaaggagtttcatcccttcattcagatgtgaacattcctgtgttccccttgacttgtctttacaccatcttaatcaatgctgtcttacagcaacaattagtcattcatagcctgtggtttgtgtccatcagtgagtgtgccctgatagacctgaaaagattgggagaccatgcccagcttcccattatttctgtagcctgtcctttgttcccataacagtacagtcatgccttagtatctgtgggggattggttctagaactccctgtggataccaaaatccatggatgctcaagtcccttatataaatggtgtagcatttgcatatgacctgtgcacatcctcgcatatattttaagtcatctctagattacttataatacttgatacaatgtaaatgatatgtaaatagttgttatactgtattgtttagggagcaaagacaagaaaaaatatatgttcagtacagataaaaccatccttttttagaaaataactttttgttcatggctggctgaatccaacaatgcaaaacccacagttagagacagccaactgtatgtgcctttgctaagtagataaatttagttaatgatgaaggactggatgaaagaaagatttgaggataaaaattagagtttgcttatctgtaaatcctgaagtaaataattacagtctctgagaaaactgagataactaatacttagtcatttacttcacatttcatgattgcttaccaagattaaacttgttctgatgaaaatcttcacttttcatactcatcgttttgatgagtgaacttaacaattaaaggtggctgggcacagcagctcatgcctataatcccagaattttgggaggctgaggcagaagaattgcttgagcccaggagttcgagaccggcctgggcaagatagggagacccccatatctacaaaaaaaatttttaaaaattagccaagtgtaggggcatatgcctgtagtcccacctactcaggaggctgaggtgggagaattgcttgaatcggggaggttgaggatgcagtgagctatgatcactccactgaactccagtctgagtgacagagtgaaaaccctgtctcaacaacagcaacaacagcaaaaacattaaaggtaattccaggacacaatagggatgaggcatattgcctgtttaaataaactttcctggccttaagaaaacaactctgctgaataattttatggaattagaatgacattaaaataatgaatctgataaatatgattagttaataaaaatacagttaaaataatacatcccttggaattttaaatcttactgagaaccatttctgcacgcacagtatccaatatacaaacacacatggaagaaaacctttccaaagctcgtggattgcaccctcatattttctgttctgttatttgagtttttaaatgctggttaccacccattatattaattttattgtattgtagcttgctcatatttttttaaaactcagcttcaaataaggcatgactaaaacagtgcatgaatggatcccttccaaatgttgaaagttattccagtttctttcctttccaagtaccatgcatatgcagctaaccctagcattggctgaccagtctaacatcagagtgaagagaagggaacttccctctacaaggcttacaaaatgataaagttacaaagataagagctaaaatgcaaacgctttctctaaagaagttaaatcctagagtcacgaactggtccatgttgcttcctttagagaaaaaagtagtaaaatcagtgcaggttgaaacctgcagcaggtacaacaggggactctattctctgtgctgtccttgaagggactccaaagatgctgtttctctgtttgcctcccagtattttaagtgagttttggattctgcaaatcaatctggctagctatggccttgggtgaatataagcaccagtttctatagccgcaagttaaagggatttgaattgttaattctgtcatccctcccattctaacatcctgttacctagaaaatcatccatacatagagcctgaaagcttacttttcaggatttggggaaataaaaaaaaatttggtagaatacctcatactttcattaggctactgatatgaacttaattattcctctaactcctttgaggctcagaccttctgtcccctgtcttatcactctcatctaatcacctccaagcaatgtcccacgtcactgagaatttagtatccaatttcctatcttctgctccactccagctcctgccttcacctggcagagtgtccatgtgggtagttgatccaataccccaagttcagagtcccttgatctcctcaatccagtgactgacacctccactccatttcagcaacttcacttctggggccacaggctgagacttgattatctccagaatttctccatgtccaaaactgaaatgggatagttcccttgaccccttcgtgggactcatgaaggggttgtcttgtttactcatcctgcagctctcaactcctcatgggagggagagcacataggtgagtgggtgcagagggtgggacaagtgcttctgggtgctggcaggagtagaactctgtgcggccccgcagcagcatctaggtgagtatctgtgacccctggagccgcagagggcatgtgttacagtgtgctcttttagctttgctgtccatggacagcttaagtgttttaacagctcagtgtgacagccctctgtatcctgaactcttgttcagcaaccaggaaggatcagcttgcacgaacaaattgaagatggtgaatgtggagaactttattgctgatgaaaatggctctcagtgggatggggagctggaaaggggatggagtgggaaggtgtctccccctggagttaggccatccccagctgaactcttctccgaggtcctaccgtcaagccatccctctgaagtcaagctgcttttcaccagtgtcaagcttcttctcttctctccttctctgccattccactgccagtggatcctggggtttttatggggacaggatggggggcagggcgggccagggtgattttggaaaaggcaacattcaggcagcaaaatggggatataaagttctcactttgggccacgggtccaggcttgagggtggagccattgccagggaccccacccttttctacctagtatttccctgcctccactctccaactctcttatcacaggcctcctatccataaatctttctcactcccttattttttttagagctcaccttcagacttatgaaagctcaagtcttttgaattcctctttccctcccagtccagcaggcttcacttctctccttgctagcctggaagtgtccattacacccttgactgtgtcgcggctgacacatggaaggcaggtgtctggtattgttaaatgaacgttaacaagtgcaacttctttaaaaagtaagatttctttatttgattgatcagttttttttttaattaagtatctaatttccattgcttctggatttaggcacacatattgaatataaaattcaaaacttttcatgtatctaaagaaataattttaatgaccacagaattttgctcttaaaacaaatggagcttgcaatataattataactaagacagactacaagcacctgctgtgttttcttcatgctgccatcaggctggtgcatagtctgatgggatggatgcatccctgctgtaataggaataagtaaagagggtgggagactgttgttggtgtttacaatcattattcatctctccattaattttcatccttgcagttgagataatcttgccttttagaacgagaaaacaatcttattacttacaacattcacaagacagggaaacactcctcaacaaagaaccatttcctttgggcatttgataagaactgctgacatctgtgtttattaagtgatgtgggagaatcagccaaggggcagaggtgtgggtggtagcttcaaaggtaatttcctgactgagtgtggttgggcatgttggcaatcccagtgtttgcaattccagtgctttgggaggctgaggcaggagcatcacttgagcccaggagtttgaggctgcagtgagctatgattacgccactgcactccagccagtgtgacagagagagaccttgtctcaaaaaaaaaaaaaaaaaggattttgctttcctggtctgtttgaatgtggtaagcacaagaacttatttgtactggtggtcacccacttgggtcctttcagtaaacttctggccatggcccagcctgagcacctgcttcctttacagagcaaagccagagccttctggtttgtaatttttgcagccacataacactgatccattttagctgtctcatgaaactgctttccaagatgaagtgagactatccgaaaccaaaatctaaaaagaatgactacagaacgaatggcagtccaaggtcatttggcagggtagaattataaggaaaggtgatggccagctactgtgaagtcttaagcccactcataggtacgaatctcttaccaggactgaagtttgtgtttccttaactttgctgcttgatggtcttctgcaaaggatctcatttctccagctttcactgggaagtggcatggggttttcgaaagtgctttaaactctaagaactggtttcctctcaggcttagccaccactagctgtctgactttggacaagttactcaaccactctgagcatcagctttctttttttaaaatggtgacagtgatatggctaccttagagagttgtggagattaagtgagataatatatgaacaaaatatataaatatgtctatattagacatatatgatctgttaaatatatttatatatatatatagaaagaatattagagaactgacatatttatcttagacctactatgttctagggcccagttctgctcatgtattatctctctatatatatagtattgagagagagaaagagagagagagctagagaaatagaaaccagtaggatatatcctatctgaagaactctaatacaggtatttcactgaacaaaatggcttctccttaaaatgtattccaacaccaatttcccctccttgcttctcatacatattaattcacagaaattatattggtcataaggcggtaggactatgaataatttttttcttctgcatttgccaaatattctgtaatgctagttttatttttataacaaatttttaaagtccctgtaaaccctataaaattaactactaatcctaaacttgaactgtaatacatatttttgttaaataaaaaaaaagatatcctaaatttgagaatctaagctaaattcacaaaatgactaaatgtatcttcttactcttaaacgataagaaaccaggaatgcagtggtttaacccaaacagcctaggtattcttcactcatttctcttgtcctcagtatatgtttcccccaggccaagaaacatttatggctaggaattcatgactgctcatctgtaagacactctccaagtactctgtcatgatttggaaccagagctttggccttccattccagatgacctccaggttctaagctttatctgaagtactccacactacattgcacaccagcatgatgggaaggaatgactcaagggaactgggcattcagaccactcaccaactacacacagcctttcacacagcagtagacaacctttgtaggaagacctctggccaaatatttatatatcaggagtatatcaatgtatggtcacagttacctcctttttaatctcaagaaataaagtcattacttctgaaaagaacaccagtaatggaactacacgggcatgccaagatgaaaattagtctctctctctctctccaccccattcttttggccactgcattttaaacaggccatagtgatagaaccagaaggtagctgtgtttcgattatttttgttttcttatttttaaacattgctttatagaagcctgttctataatgtaagaagtgaataggaatcctagtatctggaatgatgctgagccatggagatggccagattccatgtgaagagaagaggctggcagaaagctaagtatttgctcgctttccctaccctgtattcatggagcagccattacaaaagatgacgcaccagaccatgggcctggtcttgctaggtggtggagatccaaaatgctgggcactcactggagctccgtgaagatctgttgaatgagtgactaaatgtgtggtgaactgtccctacctacagggttccctgctttgcccagcctatcaggcttgggtacaagcagatagcctggattggatttcatgagggctttgtgcccactctgtatactaccggaagatgcataatctctttagacttcctgggtcatcactgaaaggccagaggcaagaatttgagcagtaaattgaagcatgtgcagagtgacatagtctgggcatagataacagtacctgacccctcaaattccctaaagttgacatgacagcttgtgcatcatggtttggtacagagatgactgaagagacagtggttgctcctctggttgctggacaccttttgaatgggctgtgatgactggagagcgtgctctgtcctggtcacctgaaggtgctcctagactagcagaagcagagacagcagccaggcctgactggagagctggagggactgggtgcaggggacttctaggccagcagtttctgtgctgtgctcggaggggctctggtggcaggaacctagtcctgggttccagctctaccagggtgcagcctgaggaactcagctttcctatttattggcttgccttgccacaaaggatttcttgtgtgttaaagatactaatagcttaaacaaacaaacaaacaaacaaacaaactattgatttaaaccactaatcaaatctttggttttacaaatttagaaaataaaattaggccccagggactaaatgtctctcctatggtccagaaaccagactgaaactgacctagtagcaaaacccattctcctgtatcccaactcccagttcaggttcctcttcattagctcccacccagctgcatagttgaggacactgaattgaggggacctggggtaaagggctggcactcaggcatcgctggcttctacatgattccaccacatggaaagcatcataatcatgtatgatccctagaaaagtgtttgctaaactccttaagttgtcattcaacaaacatttatctagcacctaccacatgctaggcaaattctagagacacagctgcccctgccctcatggaactaaatgtcttgaaagagatactggccctgatcagattttttaaaaatacataattatgcaaataaatcatattatatataattacaaagcatggtatgaaaggaaaagtacaagatgctgtgaaagcgtataaaaggaggacttggccaagtctagaggatcatcagtgagtgttccctgatggactaacattagggccaagatcagaaagatgtccaggagttaacatggtcaataatcactgttaatctttattaagcatttcccttgagtcgagtcaggcttcacttgctgcattaacttctttaatctccacaataactctgtaaggtaggtgcttttgctttttttccactttacagatgaataaacaagaagtaaaaagatgtaactaattcacatgtagccctctggcttcagaagctctttactccataaggttcctgggcacgtgggtgtggtctaggaatgggaagaggctaagaatgctgaagggcagactgaatgttccagatgaagctggcgtgataagaaagggccacatcatgtgggtcttataggttatgatattcatgttaaaactgttaatgtttttgaaatatctaaaaccaggaagtgcatgatcatatttatgttttagaaagatcactcaggctgctgtgtggagactggagtagaaagaagcatagaagagacctacagagactaggaagctgtcacagtaacctaggcacaagatagtagtggtcaagactagcttgcttgaggaagtgtagagaagtagacagataaaaggagcatccaaggagtgtacacttctaagtaatttaaaagaaagagtcaaatcccttagaagcaatcatttgaaataacctaattaccaatcctttttatccagtcctcttatgttgtcccaattcaggaattcttaaaccagttcttgtgttaaaatcatagcatggactgccttacaacattctatacttcagtccattgaaaaccacatcaggccaggtgcgtggctcatgcctataagcccagcacttttggaggccaaggcgagaggactgcttgagcccaggaattggagaccactgaactctagcctgggcaacagaatgagaccctgttgaaagagagaaaaggaagaaagaaagaaaaaagaggaaggaaggagggaaaaaaggaaaaaagggagggagggaaggtggaagaaagaaaaaaaaagaaagagaaagaaaaggctgcttcctggcacttctattaccatgtgacacgagaagggcaattgtccagacccacacttaggttcaccacagtccttggttcccaggacagagtcctttgaggttccttcaagcacccacaccatgctggaaaatgcaggctttataaactccagatgtcagtccacaactccacagccccattgggtttggttcctggatgaattctcggctagatgactggaatagagttctggtgaacatcttccttcctgccaagatgacaagtcaggtttctaaagacagatgtgtagctctttgtggaaaaaatattctgggcacgaacatcttcggctgaacggcatggaatggtgtggaaaatgtgaagccctggactggtttctgtgtcagtggtttcctgggactgccgagttgaaggggagcagaaagggctcagctgtggaggaggcagagattagccaaactagaccagctatcgccccagctgcatggatagcatcttccttcttgttcctgattaagctcatttaatatgccaatgacaaaaggatcatgaaggtctttccaaatgaatgaatgttttctggagctgagattgctggctcctatttgcatagtagtgcatactgaggcatctgaccggattactggattcctaacttttacattctttttaagctcagatatttctacggaaggcagaaagataaaggccaaaaaaagatcaggcttgcctagctcttgggtttccaggaaactgacagactcctgatatctgcaactagtgctttctctccctgggcggggcgttctccgccaccttgtggacatttggggaactgcaccgccccagcatctatgatcagaggaactgacagaggctgccataactacctggtcccctgaaataaaaaccaggcaccaggtttgcccaaaggaggaccaacacatagcagaatgaggctgagtaggatgttaaccttccatgctggtcttccattatcacgttatctgccactgtcacagacaacatctgcatccgctacagatggagatggaacaaaggctttcactggatctcaagttggatgcttctgtaaaagtaaagttccaagacctttttcttttttttccccagtgacagcaagaaaattggatgttactattggcaacaactatttcctgcttgtgaagcgttggtatttgcagtgcttctgccctgtggttattgagctgaaaggggggctgctcttgacataggagcatggcaggattggattcttcataacatgctacccagcttgtcttatgtgaaaagagaagcccagaaagagcttgggacacaaagatgcaaatcctgggtttgcatttgatttgttaagtgtcctttcacaaccagcttgcagactttaggggaaaaaaaatacatggtaggcttgtgggtagtggagaaaacataagctccagtacctcacagcctgggttttcagtctgcctcctcccaaccctcatcacataccagctgtgtgaccgtgggtactctgtctatgacaatcaattttcttgtctgcagagtacagataatagaggacctgccccatgggaagcttggaggactaaatgagattaagcccagggcctggctcatggccgattctcgtaacaggtccatgtgtgtaatcaacatgtgctgaagttctctggaccctctgtaaatatctaataggaacgactagccatgggtgtgatgagggagatgctagaaacagtaatggggtcaaatgaatctgtcccatagtctctaatttgaaacagctgaactgtgccattgtctccttggaggtaagatataagccacagagtgcctgaagcaggtctacagttaaggaacttaaatgtgctaagtgattaaaggctgggctgcctactccctgtatggcgacactctccaccctcctgcatactctccactactccaggcagtggtgggcctcctagcctctgggtgatgagcagctcccacatctcccctttgagacagaggcctgcagaacctgaccagtgtgagggctggctggggtcaggagtgtgcctgctggttggccctcaaggtgaatgtggctcttctaaaagacacctgaagggatgcagaatttagcagcaaacgccaagaacagctctttgtcatctctgcatcttctactaaagattttaagctccgaagtatatgtctggggtttctagagattgctattcccttataagggaaaaaatacacccttattgtatatttattgagcacctacaatgtgccaggcaccccagctagggagtttagatatttaatcctctcagctgccctgtaaaatagggattgtttaaaatggatttcgcccaccttataggcaaggaaactgaactacagtatggtaaagtggcttggctaatccacctggctagtgagaatggagccacagtgaacctggaggatactcttcacggcgttccatctttgtgtgccccacagcacaagcacaatgcccgggtcttaggaagagcttggtaaatgactgctgagttgagaatggtaggattcatgtaccaaaagggcatttggaaatgggcaaatgcccagatagggctctgaaagactagctatgtagagcaacagatggcaaatcatcaatttaaaactctgtcatccagccagtagcccgctggcctacagccagccatgtatgtacaagggctgacttgatcagctgtggctcttttatttcaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaaggtaagtgaagttcaggggaatgttccaccaatctaaaatttagatggctggatattttgctccttcccatttttttcctttaatgtttcaggaaaagactataatactgttgaagaatttagtttcttccttgagtttgccctcttcatggatagatttaaaacacacccaaatgccaaaagccccacttgcttcttcaagtcttctcagcatcagattccatacatcactgcttcctctgccttttaaaatcttgatcctgacatgccaaagccactctcagcttgtgtttacaggagagataactgaactaaaggggttctaattgggcagagcggggctcaagtcctggccccccaactagaggcagtgtgccttaggcaagttgcccaatctcatgggcctcagtttctttatcaaatgagggagtaccttatttatatgtctgagtctattgcgggatctggccagcagcccgcaatgcaatggggctctctctttgttcctaggcagattggcaggttgagaaataatagacacacacaagatagtgaaagctgggtccaggggggtcactgccttctggtcctgcggtgccaacaatgcactggatataccagcatttattattaagtttagtgagggcaggggtacgttagtgagggatttagggtcatttgattatgaggtgagatggtcacatggggatgaagtaattctttaacataacatttgtatgtagaagtacagtacatttgtatgtagaagtacagtatgagataagaatttacaatatagtgtgtgcgtcagtaatttctaacagagccttagaacagaaacacagtctttccataacctatgattagcaagatattaatcagcagtaacaattgcaacaaaaactggttacaaataatccatggaaacaggacatgaagctagataaccagttagaccagaaattctcagaggggagtacgccttaaccctaaagaggcctagaagagcggcggcaagatgagcgtgtttatagccctatcttatccatatggacaggccccccccatgcgtccgtttataggctccccacaagggtcacattccattcccagagctatgaacatctgcttttctgggataggaatcttggtgatgggaaacctccctgactgcacgtccatttgtaggctctctgcagggggaagcacatcacgtgctgttggctcattgtggcagtccaacctggcattgtctttacacaatcctgcatgcaattttgtatttacaataatcaggagcatttcatcttttattccgtagcaatagtttcagggcgtctccctacatgagtctcagagtaaaaatacatttttatatcattgattttcatatgtgtacagaacacactcctgtgcttgtctctcactttcccttttctgttgactatgatcatgtcaggaagatgaggtgcacagcacccaggtgagcctgtagtcacaacacagccctctcagtagccagatgggaaaggggcccaggaccacactgacagcgatagcccagctgtacctcagcaggccttttataactgcccctgggcctttcagagctcctaccgccaccactaatgctgtcccaggtgggccaatgggtagctcatacagatgtgatgagctggtagatgttcagaatattcttaaattaagatgcattccaggtcttattaccaggaaacaaaatagaagttcctaaggaaatttcatggtcaaaaccaaaatgcacacaaatatggtaaacaaatatgttctcacagcaagtaagatagatgatgtttcattccttttccagaatttagcccagtgtgagatttaattgtagggtaccttcttttctttcttctagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatggtaagtcatcagcaggagtggacatatgctagggaggaaggaaaggaagagggaagaggaaggaaggatgcgagaagggatagatcgatagctacagaatgagggatttcaaaacatttttagccagggtggtggcatgtgcctatagtcccagctactcgggaggcttaggtgggaagatggcttgagcccaggaggctgcagtgagctataattgcaccagtgcactccagcctgggcaaaagagcgagactctgtctctaaaattaaataaatatgaaaaataaataaaacattagtgcctaaacaaaagcctgatgtagcttcatactcagtaccttcttaaggtgctggaaggagatcaggtaaccaccaacatactgctttacaagaactaaaacctttattttaaggtgttcttagatgtctgaattttggatgttgctttccttttgtaagatgatgcaaatgcacaaactctgctaccctggagatatcaacataatagataaaagcatatctgtttggccagactgtttttcactcatctgttcacttgactccagagaaccctggccttgtgtgacaggaacctctttcacaggcacagaaagacaacacacagttttaatggtaataacaagtcctatggctgagcaagaccaggtatgaagaacaagcccgtctacttgtcaccaggcggaatggccatggtagctctctgggggccttactgagccaaggtctcctatgtcggactactacttccatgggctcacagcctgcacccagggaagtcatttctgtcagtgcttggcatgcattgcccagtgcccaggatacaactgcactcgataaatgtttgccgtgtaaatgacaacaatcatgctcatgccaagaccagactcatgatgtctcctcagcatcatgcagtcaatgccagagaagtagttaaggagaagctggtatataaatgttaagagaagtctggagggaaacaacccaggctgttggcagtgcttatccactctgttaaagaggctaggacaaaggagagcttttgctttttatttatttatttatttaattaattaatttatttatttattgagactgagtcttgctctgtcgcccaggctggagtgcagtggcatgatctcagctcactgcaacctccgtcccttgggttcaagcgattctcctgcttcagcctcccaagtagctgggattagaggtgtccactaccacacccagctaatttttgtatctttagtagagacggggtttcaccatgttggctagactggtctcgaactcctgaccttaggtgatccacctgccacagcctcccaaagtgctgggatttacaggtgtgagccactatgcctggccttttttttttttttttttcggctttttgtctcctaaatgagaatatattcattttatttcttataactaaacatatattttgtaaaataaaagtaagaatggatgtgattctccccagcttcaaaaggctgtctctgtctttccaaaattgccaacttgggaatcagctgcagacatagacgtggaggccaggttctctttggcatgaggtctggtgttgagcaggctctagggccaagccctcagaatcaggcagagggcatggcctgccctggagaagcctgtgcaggagtgactctgcaggccaccttgcatgggaggcccagtcccgacaccagggctacaaccgagagaccattttgctaaggccacctggcctgacagtatgggagttttaggagggggaaataagagtttgacatggactggtaacagtatatttttacttctgggagcataaaattagttctaattatagttgagagatccctgggaacacagcagcaggtgaaaagcagctttatttcctcactagagctaaccagattcctggagctgatcctaaggaagaagagccagagtaactagtgtaaatctgtgctactgttgcagattaacaacaacctgagacacacggatgaaatgttctggaaccacgtatccatgtgcattttaaatggaggtggtatccggtcgcccattgatgaacgcaacaatggtatgctcccaggcccagctcctcagtgtgtcatgttctctctctgatctccttttctgttatggttcttgccctgaacacacccatgtgcagcctcagttcatctctgctgccagccatgtaccagaatgtcagtcctttaaattagggttagccattattaaagtctaaaacctagcctgaaggacacacacaagcttgtgtgttcttatatgtattcacacacagaagctgcaggtaaggttagtggaactcaaacttggtctgaatttctgacttacaaagctatactagttgtttctgatttatgcaaaataacaacagtcaccaccttggcatctgaaaagagacaaaatcaccaatcttctgtcctgaaaaagacataccactggattaaaatgttaccttagtatatttacagatatccaagattatatcatatggcaaaacaaatcaactacacaaatactcaagacactcttgtagccctccctgatttactgtgaaaaccaatcagtatcagtataaggccccaaacgcatcccattgtcagacaacagggacaggttgcaggggcacattgtttgtctggaggaacggaacaatgcaaaacaaactactggagatccagccccaagctagcagatctaagactgagcatggtgaacttctgaggcacagccatttgtgagctcccgccatcttgcccagtggtggagtgcagggccagaggcctcctaaactcattcccttcctcagtgtcaggaaccacttagacatagctaatgatgctctataagcttcccatgtccccctcatttcttcccccagcgctgagggaaactggtcatggtgtaagcattttctcaagctattttccttcttgcctcatctgtgactaccctcaggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggtaagtcacccatcctgtagggctggcccatccaaagtgacatggcatttcctgctggttggctcagcttccccttcaccaaaattcctgtggtcaaactggtttggatttttcccgacccaacaccaataccttaccctttaatggctgtggactctcagagccactaagattaggttggggtcaaagccccaaagccccaaagccctggcaataagcagtatttgccaagtctgatttggacatcagatgggccctctaagcaggtcacctgctttgaggtctagtttctaataaaatcttatttaagcattcaattattttttttttaaatcctctcctctgttctactcatcgttgcctggaaccatgccaagatttaaaggaatgctgtccaatctgtattaaccaggatgattcctggaaggagcagcctactgcacagacatacctgaacacctggttctagtcccactgtgacctggggcaagttctcaacccctctgggcacagggagaaaacctttcctgtttatctcatggactgcagtgaaagttgaatggaaataaatgcttcctaaattgccaagggcttctgaaacacttttgttaataattccacagtccagtgaattaatcctttaaaagcaatcttattttacctggaaatcataatctggcctcatttttttaaccttaggttgtggtcccgcttgacagatagaattgctttcctgagttttcctctgcagaacacattgctgttgagactgaagggctacagaaggcagtggggccctgtcctaatgcctatggggcaacccaaacatcctccattttcccagaagggagttgagggaaaagcagttttctgatgctctagcaaagcactgcatagctctatgccttgtggaagtcaccaaatcccaaatgattaccatttaatggtcttcaaaattgtaaaatgctctctgcctctgatctgtttctcactttctaaggccattgaatcaatttagtagaccaattcagcattttaagggaaaaacagaaaaagatgtggttttgtccatgtagagtcaccctttgcctattctggtccagagctggttttccttccagtggttctcatatgtcctgtaaggactcaaggataaagggtggctgcaatgtcacaattctcttgacaaaatttcactcaggtttaaaccaaaggaaaaaccacagccccatgccccaattgtagagtttggccaaaattccttaggaaacagaaatctccctttggatctggtgaaaacagattcatttcttttctaggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtaagcatgactgtctcttcctttctctaaagaacaacaaaattgggccaagaaggggagctactagtggtgccaggagggcaaagtgactccctgtattgctgactccctcctgctttgcttgttggaaagcagcagcacagcacagcagagaggaatatgtaccctgtcaatccccgagcacacacatgccttcatccaaccccagagacaaaacatttgtaaatcaaaaatgttttaaaattcttttaactacatctaagcagctatttaaattgtgaaatataagtgagttttgcattaacagaaatatcacacctctggaattttaggatatggaaaaccatcacattaaatcatttaaattttctttcttgaagaagtttctgtgtgaatcagtcaggtagaagtggatggggtgagcgccgccctttcagcacagtgggttggtggtacaaatcataggaataaccatacttgatgtcagttggtacttctggcagcattgagggacattatatgccagcctcacttttagaaattctgttgagagtcacattatgtgcatacagaccactgacataggacacacttccccttcctcttcctagggcagagaccagcatgcttacaagacacacaagcagcttcttggcatctctgggtagttccaccaggatcttcctcagtcttatcatgctggacctgtcattttagctacttattccaaggagcgtggagggcaggaccagaccaaaggaggcagcccttcctgggaggaaaggtgctggcttcttctgtcccttccgtgacagaagatggaagggcaggcgtaaaagccccatgagacaaagaggctgatagccatcagctgtccaccactcaagcctagactgaaggatgttagagagaggagctcatgcagaaggctaaggaagccctggacctaagcgttctcaacactggttatacattagaatcacttcaggagcattttaaattaaataccaatggctcggccgcagctcttaagagattctaagttaactggattggggctaaactctttttcagtgattctagtgtacacccagaggtgaaaaccactgaagagagaagggttcttaagtactggagtgatatttttaaaataaaattctgttgggttcccaacagaacatgggttcccaacaaacatgggtttgttgtagctataggcaattagcatttatcctctcctctctttggtaaaggaaatcccccaagtcccatcatcaattacatgaaaataattattgggcaggcaaacaaaaggaagccagttttatctctcaactaattgctcttgacccagttccctaatgcccttgttctggagtggttcaaaggctatgatgtcaatgacctctttctagagtcatcacagagctgaggcaactctacttcccccaattacccaatattgaacaacagacacattttacatctggagcatgaaaaagcaatcaaattctttgtacttatgatcactagcgttcttgtcttctgtgacactttacccctgcttatgaaattgcttcccttttataattacaaaggactaccttactgttgattgataattaccttttctgtagttaaaataatgtatatgtttttctaggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttacgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccagcattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttattttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaagtcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataagctctttggtc

SEQ ID NO: 2 - DNA, 1725 bp, Homo sapiens
  source 1..1725: mol_type = genomic DNA
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 3 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 4 - DNA, 1575 bp, Homo sapiens
  source 1..1575: mol_type = genomic DNA
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 5 - protein, 524 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 6 - DNA, 250 bp, Homo sapiens
  source 1..250: mol_type = genomic DNA
gcattttctcaagctattttccttcttgcctcatctgtgactaccctcaggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggtaagtcacccatcctgtagggctggcccatccaaagtgacatggcattt

SEQ ID NO: 7 - DNA, 18 bp, synthetic construct
  source 1..18: mol_type = other DNA; note = Oligonucleotide nucleobase sequence
aatagcttgagaaaatgc

SEQ ID NO: 8 - DNA, 18 bp, synthetic construct
  source 1..18: mol_type = other DNA; note = Oligonucleotide nucleobase sequence
gaaaatagcttgagaaaa

SEQ ID NO: 9 - DNA, 18 bp, synthetic construct
  source 1..18: mol_type = other DNA; note = Oligonucleotide nucleobase sequence
aaggaaaatagcttgaga

SEQ ID NO: 10 - DNA, 18 bp, synthetic construct
  source 1..18: mol_type = other DNA; note = Oligonucleotide nucleobase sequence
aagaaggaaaatagcttg

SEQ ID NO: 11 - DNA, 18 bp, synthetic construct
  source 1..18: mol_type = other DNA; note = Oligonucleotide nucleobase sequence
ggcaagaaggaaaatagc

SEQ ID NO: 12 - DNA, 18 bp, synthetic construct
  source 1..18: mol_type = other DNA; note = Oligonucleotide nucleobase sequence
tgaggcaagaaggaaaat

SEQ ID NO: 13 - DNA, 18 bp, synthetic construct
  source 1..18: mol_type = other DNA; note = Oligonucleotide nucleobase sequence
agatgaggcaagaaggaa

SEQ ID NO: 14 - DNA, 18 bp, synthetic construct
  source 1..18: mol_type = other DNA; note = Oligonucleotide nucleobase sequence
cacagatgaggcaagaag

SEQ ID NO: 15 - DNA, 18 bp, synthetic construct
  source 1..18: mol_type = other DNA; note = Oligonucleotide nucleobase sequence
agtcacagatgaggcaag

SEQ ID NO: 16 - DNA, 18 bp, synthetic construct
  source 1..18: mol_type = other DNA; note = Oligonucleotide nucleobase sequence
ggtagtcacagatgaggc

SEQ ID NO: 17 - DNA, 18 bp, synthetic construct
  source 1..18: mol_type = other DNA; note = Oligonucleotide nucleobase sequence
gagggtagtcacagatga

SEQ ID NO: 18 - DNA, 18 bp, synthetic construct
  source 1..18: mol_type = other DNA; note = Oligonucleotide nucleobase sequence
cctgagggtagtcacaga

SEQ ID NO: 19 - DNA, 18 bp, synthetic construct
  source 1..18: mol_type = other DNA; note = Oligonucleotide nucleobase sequence
gtgcctgagggtagtcac

SEQ ID NO: 20 - DNA, 18 bp, synthetic construct
  source 1..18: mol_type = other DNA; note = Oligonucleotide nucleobase sequence
attgtgcctgagggtagt

SEQ ID NO: 21 - DNA, 18 bp, synthetic construct
  source 1..18: mol_type = other DNA; note = Oligonucleotide nucleobase sequence
gtaattgtgcctgagggt

SEQ ID NO: 22 - DNA, 18 bp, synthetic construct
  source 1..18: mol_type = other DNA; note = Oligonucleotide nucleobase sequence
caggtaattgtgcctgag

SEQ ID NO: 23 - DNA, 18 bp, synthetic construct
  source 1..18: mol_type = other DNA; note = Oligonucleotide nucleobase sequence
tcccaggtaattgtgcct

SEQ ID NO: 24 - DNA, 18 bp, synthetic construct
  source 1..18: mol_type = other DNA; note = Oligonucleotide nucleobase sequence
ttctcccaggtaattgtg

SEQ ID NO: 25 - DNA, 18 bp, synthetic construct
  source 1..18: mol_type = other DNA; note = Oligonucleotide nucleobase sequence
aggttctcccaggtaatt

SEQ ID NO: 26 - DNA, 18 bp, synthetic construct
  source 1..18: mol_type = other DNA; note = Oligonucleotide nucleobase sequence
gccaggttctcccaggta

SEQ ID NO: 27 - DNA, 18 bp, synthetic construct
  source 1..18: mol_type = other DNA; note = Oligonucleotide nucleobase sequence
gcagccaggttctcccag

SEQ ID NO: 28 - DNA, 18 bp, synthetic construct
  source 1..18: mol_type = other DNA; note = Oligonucleotide nucleobase sequence
acagcagccaggttctcc

SEQ ID NO: 29 - DNA, 18 bp, synthetic construct
  source 1..18: mol_type = other DNA; note = Oligonucleotide nucleobase sequence
aatacagcagccaggttc

SEQ ID NO: 30 - DNA, 18 bp, synthetic construct
  source 1..18: mol_type = other DNA; note = Oligonucleotide nucleobase sequence
ggcaatacagcagccagg

SEQ ID NO: 31 - DNA, 18 bp, synthetic construct
  source 1..18: mol_type = other DNA; note = Oligonucleotide nucleobase sequence
aagggcaatacagcagcc

SEQ ID NO: 32 - DNA, 18 bp, synthetic construct
  source 1..18: mol_type = other DNA; note = Oligonucleotide nucleobase sequence
ccaaagggcaatacagca

SEQ ID NO: 33 - DNA, 18 bp, synthetic construct
  source 1..18: mol_type = other DNA; note = Oligonucleotide nucleobase sequence
cctccaaagggcaataca

SEQ ID NO: 34 - DNA, 18 bp, synthetic construct
  source 1..18: mol_type = other DNA; note = Oligonucleotide nucleobase sequence
gtgcctccaaagggcaat

SEQ ID NO: 35 - DNA, 18 bp, synthetic construct
  source 1..18: mol_type = other DNA; note = Oligonucleotide nucleobase sequence
aatgtgcctccaaagggc

SEQ ID NO: 36 - DNA, 18 bp, synthetic construct
  source 1..18: mol_type = other DNA; note = Oligonucleotide nucleobase sequence
tcaaatgtgcctccaaag

SEQ ID NO: 37 - DNA, 18 bp, synthetic construct
  source 1..18: mol_type = other DNA; note = Oligonucleotide nucleobase sequence
aggtcaaatgtgcctcca

SEQ ID NO: 38 - DNA, 18 bp, synthetic construct
  source 1..18: mol_type = other DNA; note = Oligonucleotide nucleobase sequence
actaggtcaaatgtgcct

SEQ ID NO: 39 - DNA, 18 bp, synthetic construct
  source 1..18: mol_type = other DNA; note = Oligonucleotide nucleobase sequence
tggactaggtcaaatgtg

SEQ ID NO: 40 - DNA, 18 bp, synthetic construct
  source 1..18: mol_type = other DNA; note = Oligonucleotide nucleobase sequence
aactggactaggtcaaat

SEQ ID NO: 41 - DNA, 18 bp, synthetic construct
  source 1..18: mol_type = other DNA; note = Oligonucleotide nucleobase sequence
tttaactggactaggtca

SEQ ID NO: 42 - DNA, 18 bp, synthetic construct
  source 1..18: mol_type = other DNA; note = Oligonucleotide nucleobase sequence
ccttttaactggactagg

SEQ ID NO: 43 - DNA, 18 bp, synthetic construct
  source 1..18: mol_type = other DNA; note = Oligonucleotide nucleobase sequence
gaaccttttaactggact

SEQ ID NO: 44 - DNA, 18 bp, synthetic construct
  source 1..18: mol_type = other DNA; note = Oligonucleotide nucleobase sequence
gtggaaccttttaactgg

SEQ ID NO: 45 - DNA, 18 bp, synthetic construct
  source 1..18: mol_type = other DNA; note = Oligonucleotide nucleobase sequence
agggtggaaccttttaac

SEQ ID NO: 46 - DNA, 18 bp, synthetic construct
  source 1..18: mol_type = other DNA; note = Oligonucleotide nucleobase sequence
ttcagggtggaacctttt

SEQ ID NO: 47 - DNA, 18 bp, synthetic construct
  source 1..18: mol_type = other DNA; note = Oligonucleotide nucleobase sequence
ttcttcagggtggaacct

SEQ ID NO: 48 - DNA, 18 bp, synthetic construct
  source 1..18: mol_type = other DNA; note = Oligonucleotide nucleobase sequence
gccttcttcagggtggaa

SEQ ID NO: 49 - DNA, 18 bp, synthetic construct
  source 1..18: mol_type = other DNA; note = Oligonucleotide nucleobase sequence
aaggccttcttcagggtg

SEQ ID NO: 50 - DNA, 18 bp, synthetic construct
  source 1..18: mol_type = other DNA; note = Oligonucleotide nucleobase sequence
tcaaaggccttcttcagg

SEQ ID NO: 51 - DNA, 18 bp, synthetic construct
  source 1..18: mol_type = other DNA; note = Oligonucleotide nucleobase sequence
tgctcaaaggccttcttc

SEQ ID NO: 52 - DNA, 18 bp, synthetic construct
  source 1..18: mol_type = other DNA; note = Oligonucleotide nucleobase sequence
ctatgctcaaaggccttc

SEQ ID NO: 53 - DNA, 18 bp, synthetic construct
  source 1..18: mol_type = other DNA; note = Oligonucleotide nucleobase sequence
acgctatgctcaaaggcc

SEQ ID NO: 54 - DNA, 18 bp, synthetic construct
  source 1..18: mol_type = other DNA; note = Oligonucleotide nucleobase sequence
tgcacgctatgctcaaag

SEQ ID NO: 55 - DNA, 18 bp, synthetic construct
  source 1..18: mol_type = other DNA; note = Oligonucleotide nucleobase sequence
cggtgcacgctatgctca

SEQ ID NO: 56 - DNA, 18 bp, synthetic construct
  source 1..18: mol_type = other DNA; note = Oligonucleotide nucleobase sequence
tagcggtgcacgctatgc

SEQ ID NO: 57 - DNA, 18 bp, synthetic construct
  source 1..18: mol_type = other DNA; note = Oligonucleotide nucleobase sequence
ccgtagcggtgcacgcta

SEQ ID NO: 58 - DNA, 18 bp, synthetic construct
  source 1..18: mol_type = other DNA; note = Oligonucleotide nucleobase sequence
tggccgtagcggtgcacg

SEQ ID NO: 59 - DNA, 18 bp, synthetic construct
  source 1..18: mol_type = other DNA; note = Oligonucleotide nucleobase sequence
gactggccgtagcggtgc

SEQ ID NO: 60 - DNA, 18 bp, synthetic construct
  source 1..18: mol_type = other DNA; note = Oligonucleotide nucleobase sequence
gtggactggccgtagcgg

SEQ ID NO: 61 - DNA, 18 bp, synthetic construct
  source 1..18: mol_type = other DNA; note = Oligonucleotide nucleobase sequence
ccagtggactggccgtag

SEQ ID NO: 62 - DNA, 18 bp, synthetic construct
  source 1..18: mol_type = other DNA; note = Oligonucleotide nucleobase sequence
tctccagtggactggccg

SEQ ID NO: 63 - DNA, 18 bp, synthetic construct
  source 1..18: mol_type = other DNA; note = Oligonucleotide nucleobase sequence
aactctccagtggactgg

SEQ ID NO: 64 - DNA, 18 bp, synthetic construct
  source 1..18: mol_type = other DNA; note = Oligonucleotide nucleobase sequence
aggaactctccagtggac

SEQ ID NO: 65 - DNA, 18 bp, synthetic construct
  source 1..18: mol_type = other DNA; note = Oligonucleotide nucleobase sequence
tgcaggaactctccagtg

SEQ ID NO: 66 - DNA, 18 bp, synthetic construct
  source 1..18: mol_type = other DNA; note = Oligonucleotide nucleobase sequence
acctgcaggaactctcca

SEQ ID NO: 67 - DNA, 18 bp, synthetic construct
  source 1..18: mol_type = other DNA; note = Oligonucleotide nucleobase sequence
cccacctgcaggaactct

SEQ ID NO: 68 - DNA, 18 bp, synthetic construct
  source 1..18: mol_type = other DNA; note = Oligonucleotide nucleobase sequence
ccgcccacctgcaggaac

SEQ ID NO: 69 - DNA, 18 bp, synthetic construct
  source 1..18: mol_type = other DNA; note = Oligonucleotide nucleobase sequence
ttaccgcccacctgcagg

SEQ ID NO: 70 - DNA, 18 bp, synthetic construct
  source 1..18: mol_type = other DNA; note = Oligonucleotide nucleobase sequence
gacttaccgcccacctgc

SEQ ID NO: 71 - DNA, 18 bp, synthetic construct
  source 1..18: mol_type = other DNA; note = Oligonucleotide nucleobase sequence
ggtgacttaccgcccacc

SEQ ID NO: 72 - DNA, 18 bp, synthetic construct
  source 1..18: mol_type = other DNA; note = Oligonucleotide nucleobase sequence
atgggtgacttaccgccc

SEQ ID NO: 73 - DNA, 18 bp, synthetic construct
  source 1..18: mol_type = other DNA; note = Oligonucleotide nucleobase sequence
aggatgggtgacttaccg

SEQ ID NO: 74 - DNA, 18 bp, synthetic construct
  source 1..18: mol_type = other DNA; note = Oligonucleotide nucleobase sequence
tacaggatgggtgactta

SEQ ID NO: 75 - DNA, 18 bp, synthetic construct
  source 1..18: mol_type = other DNA; note = Oligonucleotide nucleobase sequence
ccctacaggatgggtgac

SEQ ID NO: 76 - DNA, 18 bp, synthetic construct
  source 1..18: mol_type = other DNA; note = Oligonucleotide nucleobase sequence
cagccctacaggatgggt

SEQ ID NO: 77 - DNA, 18 bp, synthetic construct
  source 1..18: mol_type = other DNA; note = Oligonucleotide nucleobase sequence
ggccagccctacaggatg

SEQ ID NO: 78 - DNA, 18 bp, synthetic construct
  source 1..18: mol_type = other DNA; note = Oligonucleotide nucleobase sequence
atgggccagccctacagg

SEQ ID NO: 79 - DNA, 18 bp, synthetic construct
  source 1..18: mol_type = other DNA; note = Oligonucleotide nucleobase sequence
tggatgggccagccctac

SEQ ID NO: 80 - DNA, 18 bp, synthetic construct
  source 1..18: mol_type = other DNA; note = Oligonucleotide nucleobase sequence
ctttggatgggccagccc

SEQ ID NO: 81 - DNA, 18 bp, synthetic construct
  source 1..18: mol_type = other DNA; note = Oligonucleotide nucleobase sequence
tcactttggatgggccag

SEQ ID NO: 82 - DNA, 18 bp, synthetic construct
  source 1..18: mol_type = other DNA; note = Oligonucleotide nucleobase sequence
atgtcactttggatgggc

SEQ ID NO: 83 - DNA, 18 bp, synthetic construct
  source 1..18: mol_type = other DNA; note = Oligonucleotide nucleobase sequence
gccatgtcactttggatg

SEQ ID NO: 84 - DNA, 18 bp, synthetic construct
  source 1..18: mol_type = other DNA; note = Oligonucleotide nucleobase sequence
aatgccatgtcactttgg

SEQ ID NO: 85 - DNA, 20 bp, synthetic construct
  source 1..20: mol_type = other DNA; note = Oligonucleotide nucleobase sequence
ccaggtaattgtgcctgagg

SEQ ID NO: 86 - DNA, 20 bp, synthetic construct
  source 1..20: mol_type = other DNA; note = Oligonucleotide nucleobase sequence
cccaggtaattgtgcctgag

SEQ ID NO: 87 - DNA, 20 bp, synthetic construct
  source 1..20: mol_type = other DNA; note = Oligonucleotide nucleobase sequence
tcccaggtaattgtgcctga

SEQ ID NO: 88 - DNA, 20 bp, synthetic construct
  source 1..20: mol_type = other DNA; note = Oligonucleotide nucleobase sequence
ctcccaggtaattgtgcctg

SEQ ID NO: 89 - DNA, 16 bp, synthetic construct
  source 1..16: mol_type = other DNA; note = Oligonucleotide nucleobase sequence
gatttcccagtgccat

SEQ ID NO: 90 - DNA, 16 bp, synthetic construct
  source 1..16: mol_type = other DNA; note = Oligonucleotide nucleobase sequence
gcactcgacacttggt

SEQ ID NO: 91 - DNA, 18 bp, synthetic construct
  source 1..18: mol_type = other DNA; note = Oligonucleotide nucleobase sequence
gtacatccacttattcaa

SEQ ID NO: 92 - DNA, 20 bp, synthetic construct
  source 1..20: mol_type = other DNA; note = Oligonucleotide nucleobase sequence
ccaaatcttataataactac

SEQ ID NO: 93 - DNA, 18 bp, Homo sapiens
  source 1..18: mol_type = genomic DNA
gcattttctcaagctatt

SEQ ID NO: 94 - DNA, 18 bp, Homo sapiens
  source 1..18: mol_type = genomic DNA
ttttctcaagctattttc

SEQ ID NO: 95 - DNA, 18 bp, Homo sapiens
  source 1..18: mol_type = genomic DNA
tctcaagctattttcctt

SEQ ID NO: 96 - DNA, 18 bp, Homo sapiens
  source 1..18: mol_type = genomic DNA
caagctattttccttctt

SEQ ID NO: 97 - DNA, 18 bp, Homo sapiens
  source 1..18: mol_type = genomic DNA
gctattttccttcttgcc

SEQ ID NO: 98 - DNA, 18 bp, Homo sapiens
  source 1..18: mol_type = genomic DNA
attttccttcttgcctca

SEQ ID NO: 99 - DNA, 18 bp, Homo sapiens
  source 1..18: mol_type = genomic DNA
ttccttcttgcctcatct

SEQ ID NO: 100 - DNA, 18 bp, Homo sapiens
  source 1..18: mol_type = genomic DNA
cttcttgcctcatctgtg

SEQ ID NO: 101 - DNA, 18 bp, Homo sapiens
  source 1..18: mol_type = genomic DNA
cttgcctcatctgtgact

SEQ ID NO: 102 - DNA, 18 bp, Homo sapiens
  source 1..18: mol_type = genomic DNA
gcctcatctgtgactacc

SEQ ID NO: 103 - DNA, 18 bp, Homo sapiens
  source 1..18: mol_type = genomic DNA
tcatctgtgactaccctc

SEQ ID NO: 104 - DNA, 18 bp, Homo sapiens
  source 1..18: mol_type = genomic DNA
tctgtgactaccctcagg

SEQ ID NO: 105 - DNA, 18 bp, Homo sapiens
  source 1..18: mol_type = genomic DNA
gtgactaccctcaggcac

SEQ ID NO: 106 - DNA, 18 bp, Homo sapiens
  source 1..18: mol_type = genomic DNA
actaccctcaggcacaat

SEQ ID NO: 107 - DNA, 18 bp, Homo sapiens
  source 1..18: mol_type = genomic DNA
accctcaggcacaattac

SEQ ID NO: 108 - DNA, 18 bp, Homo sapiens
  source 1..18: mol_type = genomic DNA
ctcaggcacaattacctg

SEQ ID NO: 109 - DNA, 18 bp, Homo sapiens
  source 1..18: mol_type = genomic DNA
aggcacaattacctggga

SEQ ID NO: 110 - DNA, 18 bp, Homo sapiens
  source 1..18: mol_type = genomic DNA
cacaattacctgggagaa

SEQ ID NO: 111 - DNA, 18 bp, Homo sapiens
  source 1..18: mol_type = genomic DNA
aattacctgggagaacct

SEQ ID NO: 112 - DNA, 18 bp, Homo sapiens
  source 1..18: mol_type = genomic DNA
tacctgggagaacctggc

SEQ ID NO: 113 - DNA, 18 bp, Homo sapiens
  source 1..18: mol_type = genomic DNA
ctgggagaacctggctgc

SEQ ID NO: 114 - DNA, 18 bp, Homo sapiens
  source 1..18: mol_type = genomic DNA
ggagaacctggctgctgt

SEQ ID NO: 115 - DNA, 18 bp, Homo sapiens
  source 1..18: mol_type = genomic DNA
gaacctggctgctgtatt

SEQ ID NO: 116 - DNA, 18 bp, Homo sapiens
  source 1..18: mol_type = genomic DNA
cctggctgctgtattgcc

SEQ ID NO: 117 - DNA, 18 bp, Homo sapiens
  source 1..18: mol_type = genomic DNA
ggctgctgtattgccctt

SEQ ID NO: 118 - DNA, 18 bp, Homo sapiens
  source 1..18: mol_type = genomic DNA
tgctgtattgccctttgg

SEQ ID NO: 119 - DNA, 18 bp, Homo sapiens
  source 1..18: mol_type = genomic DNA
tgtattgccctttggagg

SEQ ID NO: 120 - DNA, 18 bp, Homo sapiens
  source 1..18: mol_type = genomic DNA
attgccctttggaggcac

SEQ ID NO: 121 - DNA, 18 bp, Homo sapiens
  source 1..18: mol_type = genomic DNA
gccctttggaggcacatt

SEQ ID NO: 122 - DNA, 18 bp, Homo sapiens
  source 1..18: mol_type = genomic DNA
ctttggaggcacatttga

SEQ ID NO: 123 - DNA, 18 bp, Homo sapiens
  source 1..18: mol_type = genomic DNA
tggaggcacatttgacct

SEQ ID NO: 124 - DNA, 18 bp, Homo sapiens
  source 1..18: mol_type = genomic DNA
aggcacatttgacctagt

SEQ ID NO: 125 - DNA, 18 bp, Homo sapiens
  source 1..18: mol_type = genomic DNA
cacatttgacctagtcca

SEQ ID NO: 126 - DNA, 18 bp, Homo sapiens
  source 1..18: mol_type = genomic DNA
atttgacctagtccagtt

SEQ ID NO: 127 - DNA, 18 bp, Homo sapiens
  source 1..18: mol_type = genomic DNA
tgacctagtccagttaaa

SEQ ID NO: 128 - DNA, 18 bp, Homo sapiens
  source 1..18: mol_type = genomic DNA
cctagtccagttaaaagg

SEQ ID NO: 129 - DNA, 18 bp, Homo sapiens
  source 1..18: mol_type = genomic DNA
agtccagttaaaaggttc

SEQ ID NO: 130 - DNA, 18 bp, Homo sapiens
  source 1..18: mol_type = genomic DNA
ccagttaaaaggttccac

SEQ ID NO: 131 - DNA, 18 bp, Homo sapiens
  source 1..18: mol_type = genomic DNA
gttaaaaggttccaccct

SEQ ID NO: 132 - DNA, 18 bp, Homo sapiens
  source 1..18: mol_type = genomic DNA
aaaaggttccaccctgaa

SEQ ID NO: 133 - DNA, 18 bp, Homo sapiens
  source 1..18: mol_type = genomic DNA
aggttccaccctgaagaa

SEQ ID NO: 134 - DNA, 18 bp, Homo sapiens
  source 1..18: mol_type = genomic DNA
ttccaccctgaagaaggc

SEQ ID NO: 135 - DNA, 18 bp, Homo sapiens
  source 1..18: mol_type = genomic DNA
caccctgaagaaggcctt

SEQ ID NO: 136 - DNA, 18 bp, Homo sapiens
  source 1..18: mol_type = genomic DNA
cctgaagaaggcctttga

SEQ ID NO: 137 - DNA, 18 bp, Homo sapiens
  source 1..18: mol_type = genomic DNA
gaagaaggcctttgagca

SEQ ID NO: 138 - DNA, 18 bp, Homo sapiens
  source 1..18: mol_type = genomic DNA
gaaggcctttgagcatag

SEQ ID NO: 139 - DNA, 18 bp, Homo sapiens
  source 1..18: mol_type = genomic DNA
ggcctttgagcatagcgt

SEQ ID NO: 140 - DNA, 18 bp, Homo sapiens
  source 1..18: mol_type = genomic DNA
ctttgagcatagcgtgca

SEQ ID NO: 141 - DNA, 18 bp, Homo sapiens
  source 1..18: mol_type = genomic DNA
tgagcatagcgtgcaccg

SEQ ID NO: 142 - DNA, 18 bp, Homo sapiens
  source 1..18: mol_type = genomic DNA
gcatagcgtgcaccgcta

SEQ ID NO: 143 - DNA, 18 bp, Homo sapiens
  source 1..18: mol_type = genomic DNA
tagcgtgcaccgctacgg

SEQ ID NO: 144 - DNA, 18 bp, Homo sapiens
  source 1..18: mol_type = genomic DNA
cgtgcaccgctacggcca

SEQ ID NO: 145 - DNA, 18 bp, Homo sapiens
  source 1..18: mol_type = genomic DNA
gcaccgctacggccagtc

SEQ ID NO: 146 - DNA, 18 bp, Homo sapiens
  source 1..18: mol_type = genomic DNA
ccgctacggccagtccac

SEQ ID NO: 147 - DNA, 18 bp, Homo sapiens
  source 1..18: mol_type = genomic DNA
ctacggccagtccactgg

SEQ ID NO: 148 - DNA, 18 bp, Homo sapiens
  source 1..18: mol_type = genomic DNA
cggccagtccactggaga

SEQ ID NO: 149 - DNA, 18 bp, Homo sapiens
  source 1..18: mol_type = genomic DNA
ccagtccactggagagtt

SEQ ID NO: 150 - DNA, 18 bp, Homo sapiens
  source 1..18: mol_type = genomic DNA
gtccactggagagttcct

SEQ ID NO: 151 - DNA, 18 bp, Homo sapiens
  source 1..18: mol_type = genomic DNA
cactggagagttcctgca

SEQ ID NO: 152 - DNA, 18 bp, Homo sapiens
  source 1..18: mol_type = genomic DNA
tggagagttcctgcaggt

SEQ ID NO: 153 - DNA, 18 bp, Homo sapiens
  source 1..18: mol_type = genomic DNA
agagttcctgcaggtggg

SEQ ID NO: 154 - DNA, 18 bp, Homo sapiens
  source 1..18: mol_type = genomic DNA
gttcctgcaggtgggcgg

SEQ ID NO: 155 - DNA, 18 bp, Homo sapiens
  source 1..18: mol_type = genomic DNA
cctgcaggtgggcggtaa

SEQ ID NO: 156 - DNA, 18 bp, Homo sapiens
  source 1..18: mol_type = genomic DNA
gcaggtgggcggtaagtc

SEQ ID NO: 157 - DNA, 18 bp, Homo sapiens
  source 1..18: mol_type = genomic DNA
ggtgggcggtaagtcacc

SEQ ID NO: 158 - DNA, 18 bp, Homo sapiens
  source 1..18: mol_type = genomic DNA
gggcggtaagtcacccat

SEQ ID NO: 159 - DNA, 18 bp, Homo sapiens
  source 1..18: mol_type = genomic DNA
cggtaagtcacccatcct

SEQ ID NO: 160 - DNA, 18 bp, Homo sapiens
  source 1..18: mol_type = genomic DNA
taagtcacccatcctgta

SEQ ID NO: 161 - DNA, 18 bp, Homo sapiens
  source 1..18: mol_type = genomic DNA
gtcacccatcctgtaggg

SEQ ID NO: 162 - DNA, 18 bp, Homo sapiens
  source 1..18: mol_type = genomic DNA
acccatcctgtagggctg

SEQ ID NO: 163 - DNA, 18 bp, Homo sapiens
  source 1..18: mol_type = genomic DNA
catcctgtagggctggcc

SEQ ID NO: 164 - DNA, 18 bp, Homo sapiens
  source 1..18: mol_type = genomic DNA
cctgtagggctggcccat

SEQ ID NO: 165 - DNA, 18 bp, Homo sapiens
  source 1..18: mol_type = genomic DNA
gtagggctggcccatcca

SEQ ID NO: 166 - DNA, 18 bp, Homo sapiens
  source 1..18: mol_type = genomic DNA
gggctggcccatccaaag

SEQ ID NO: 167 - DNA, 18 bp, Homo sapiens
  source 1..18: mol_type = genomic DNA
ctggcccatccaaagtga

SEQ ID NO: 168 - DNA, 18 bp, Homo sapiens
  source 1..18: mol_type = genomic DNA
gcccatccaaagtgacat

SEQ ID NO: 169 - DNA, 18 bp, Homo sapiens
  source 1..18: mol_type = genomic DNA
catccaaagtgacatggc

SEQ ID NO: 170 - DNA, 18 bp, Homo sapiens
  source 1..18: mol_type = genomic DNA
ccaaagtgacatggcatt

SEQ ID NO: 171 - DNA, 20 bp, Homo sapiens
  source 1..20: mol_type = genomic DNA
cctcaggcacaattacctgg

SEQ ID NO: 172 - DNA, 20 bp, Homo sapiens
  source 1..20: mol_type = genomic DNA
ctcaggcacaattacctggg

SEQ ID NO: 173 - DNA, 20 bp, Homo sapiens
  source 1..20: mol_type = genomic DNA
tcaggcacaattacctggga

SEQ ID NO: 174 - DNA, 20 bp, Homo sapiens
  source 1..20: mol_type = genomic DNA
caggcacaattacctgggag

SEQ ID NO: 175 - DNA, 16 bp, Homo sapiens
  source 1..16: mol_type = genomic DNA
atggcactgggaaatc

SEQ ID NO: 176 - DNA, 16 bp, Homo sapiens
  source 1..16: mol_type = genomic DNA
accaagtgtcgagtgc

SEQ ID NO: 177 - DNA, 18 bp, Homo sapiens
  source 1..18: mol_type = genomic DNA
ttgaataagtggatgtac

SEQ ID NO: 178 - DNA, 20 bp, Homo sapiens
  source 1..20: mol_type = genomic DNA
gtagttattataagatttgg

SEQ ID NO: 179 - DNA, 17 bp, synthetic construct
  source 1..17: mol_type = other DNA; note = Primer
ggaggtggtatccggtc

SEQ ID NO: 180 - DNA, 18 bp, synthetic construct
  source 1..18: mol_type = other DNA; note = Primer
ccgaaaacctggagacag

SEQ ID NO: 181 - DNA, 26 bp, synthetic construct
  source 1..26: mol_type = other DNA; note = Primer
aacgcaacaatggaatccatgtggtg

SEQ ID NO: 182 - DNA, 24 bp, synthetic construct
  source 1..24: mol_type = other DNA; note = Primer
aacgcaacaatggcacaattacct

SEQ ID NO: 183 - DNA, 21 bp, synthetic construct
  source 1..21: mol_type = other DNA; note = Primer
gcgatgtcaataggactccag

SEQ ID NO: 184 - DNA, 22 bp, synthetic construct
  source 1..22: mol_type = other DNA; note = Primer
ttgttgtaggatatgcccttga

SEQ ID NO: 185 - DNA, 31 bp, synthetic construct
  source 1..31: mol_type = other DNA; note = Primer
agcctaagatgagagttcaagttgagtttgg

SEQ ID NO: 186 - DNA, 31 bp, synthetic construct; identical to SEQ ID NO: 185
  source 1..31: mol_type = other DNA; note = Primer

SEQ ID NO: 187 - DNA, 17 bp, synthetic construct
  source 1..17: mol_type = other DNA
  misc_feature 1: note = adenine attached to 56-FAM-aacgcaaca-ZEN quencher
  modified_base 17: mod_base = OTHER; note = guanine-3 prime Iowa Black FQ
atggaatccatgtggtg

SEQ ID NO: 188 - DNA, 15 bp, synthetic construct
  source 1..15: mol_type = other DNA
  misc_feature 1: note = adenine attached to hexachlorofluorescein-aacgcaaca-ZEN quencher
  modified_base 15: mod_base = OTHER; note = thymine-3 prime Iowa Black FQ
atggcacaattacct

SEQ ID NO: 189 - DNA, 22 bp, synthetic construct
  source 1..22: mol_type = other DNA
  misc_feature 1: note = thymine attached to hexachlorofluorescein-agcctaaga-ZEN quencher
  modified_base 22: mod_base = OTHER; note = guanine-3 prime Iowa Black FQ
tgagagttcaagttgagtttgg

SEQ ID NO: 190 - DNA, 22 bp, synthetic construct
  source 1..22: mol_type = other DNA
  misc_feature 1: note = thymine attached to 56-FAM-agcctaaga-ZEN quencher
  modified_base 22: mod_base = OTHER; note = guanine-3 prime Iowa Black FQ
tgagagttcaagttgagtttgg

SEQ ID NO: 191 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = beta-D-oxy LNA thymine phosphorothioate
  modified_base 4: mod_base = OTHER; note = adenine phosphorothioate
  modified_base 5: mod_base = OTHER; note = beta-D-oxy LNA guanine phosphorothioate
  modified_base 6: mod_base = OTHER; note = beta-D-oxy LNA cytosine phosphorothioate
  modified_base 7: mod_base = OTHER; note = thymine phosphorothioate
  modified_base 8: mod_base = OTHER; note = beta-D-oxy LNA thymine phosphorothioate
  modified_base 9: mod_base = OTHER; note = beta-D-oxy LNA guanine phosphorothioate
  modified_base 10: mod_base = OTHER; note = adenine phosphorothioate
  modified_base 11: mod_base = OTHER; note = beta-D-oxy LNA guanine phosphorothioate
  modified_base 12: mod_base = OTHER; note = beta-D-oxy LNA adenine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA adenine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA cytosine
aatagcttgagaaaatgc

SEQ ID NO: 192 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = beta-D-oxy LNA adenine phosphorothioate
  modified_base 3: mod_base = OTHER; note = adenine phosphorothioate
  modified_base 4: mod_base = OTHER; note = adenine phosphorothioate
  modified_base 5: mod_base = OTHER; note = beta-D-oxy LNA adenine phosphorothioate
  modified_base 6: mod_base = OTHER; note = beta-D-oxy LNA thymine phosphorothioate
  modified_base 7: mod_base = OTHER; note = adenine phosphorothioate
  modified_base 8: mod_base = OTHER; note = beta-D-oxy LNA guanine phosphorothioate
  modified_base 9: mod_base = OTHER; note = beta-D-oxy LNA cytosine phosphorothioate
  modified_base 10: mod_base = OTHER; note = thymine phosphorothioate
  modified_base 11: mod_base = OTHER; note = beta-D-oxy LNA thymine phosphorothioate
  modified_base 12: mod_base = OTHER; note = beta-D-oxy LNA guanine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = beta-D-oxy LNA adenine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA adenine
gaaaatagcttgagaaaa

SEQ ID NO: 193 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = beta-D-oxy LNA guanine phosphorothioate
  modified_base 4: mod_base = OTHER; note = beta-D-oxy LNA guanine phosphorothioate
  modified_base 5: mod_base = OTHER; note = adenine phosphorothioate
  modified_base 6: mod_base = OTHER; note = beta-D-oxy LNA adenine phosphorothioate
  modified_base 7: mod_base = OTHER; note = adenine phosphorothioate
  modified_base 8: mod_base = OTHER; note = beta-D-oxy LNA adenine phosphorothioate
  modified_base 9: mod_base = OTHER; note = beta-D-oxy LNA thymine phosphorothioate
  modified_base 10: mod_base = OTHER; note = adenine phosphorothioate
  modified_base 11: mod_base = OTHER; note = beta-D-oxy LNA guanine phosphorothioate
  modified_base 12: mod_base = OTHER; note = beta-D-oxy LNA cytosine phosphorothioate
  modified_base 13: mod_base = OTHER; note = thymine phosphorothioate
  modified_base 14: mod_base = OTHER; note = beta-D-oxy LNA thymine phosphorothioate
  modified_base 15: mod_base = OTHER; note = beta-D-oxy LNA guanine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA adenine
aaggaaaatagcttgaga

SEQ ID NO: 194 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = beta-D-oxy LNA guanine phosphorothioate
  modified_base 4: mod_base = OTHER; note = beta-D-oxy LNA adenine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = beta-D-oxy LNA guanine phosphorothioate
  modified_base 8: mod_base = OTHER; note = adenine phosphorothioate
  modified_base 9: mod_base = OTHER; note = beta-D-oxy LNA adenine phosphorothioate
  modified_base 10: mod_base = OTHER; note = adenine phosphorothioate
  modified_base 11: mod_base = OTHER; note = beta-D-oxy LNA adenine phosphorothioate
  modified_base 12: mod_base = OTHER; note = beta-D-oxy LNA thymine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = beta-D-oxy LNA cytosine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA guanine
aagaaggaaaatagcttg

SEQ ID NO: 195 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = guanine phosphorothioate
  modified_base 3: mod_base = OTHER; note = beta-D-oxy LNA cytosine phosphorothioate
  modified_base 4: mod_base = OTHER; note = beta-D-oxy LNA adenine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = beta-D-oxy LNA guanine phosphorothioate
  modified_base 10: mod_base = OTHER; note = beta-D-oxy LNA guanine phosphorothioate
  modified_base 11: mod_base = OTHER; note = adenine phosphorothioate
  modified_base 12: mod_base = OTHER; note = beta-D-oxy LNA adenine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA thymine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA cytosine
ggcaagaaggaaaatagc

SEQ ID NO: 196 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = beta-D-oxy LNA adenine phosphorothioate
  modified_base 4: mod_base = OTHER; note = beta-D-oxy LNA guanine phosphorothioate
  modified_base 5: mod_base = OTHER; note = guanine phosphorothioate
  modified_base 6: mod_base = OTHER; note = beta-D-oxy LNA cytosine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = beta-D-oxy LNA guanine phosphorothioate
  modified_base 10: mod_base = OTHER; note = beta-D-oxy LNA adenine phosphorothioate
  modified_base 11: mod_base = OTHER; note = adenine phosphorothioate
  modified_base 12: mod_base = OTHER; note = beta-D-oxy LNA guanine phosphorothioate
  modified_base 13: mod_base = OTHER; note = beta-D-oxy LNA guanine phosphorothioate
  modified_base 14: mod_base = OTHER; note = adenine phosphorothioate
  modified_base 15: mod_base = OTHER; note = beta-D-oxy LNA adenine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA thymine
tgaggcaagaaggaaaat

SEQ ID NO: 197 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = guanine phosphorothioate
  modified_base 3: mod_base = OTHER; note = beta-D-oxy LNA adenine phosphorothioate
  modified_base 4: mod_base = OTHER; note = beta-D-oxy LNA thymine phosphorothioate
  modified_base 5: mod_base = OTHER; note = guanine phosphorothioate
  modified_base 6: mod_base = OTHER; note = beta-D-oxy LNA adenine phosphorothioate
  modified_base 7: mod_base = OTHER; note = beta-D-oxy LNA guanine phosphorothioate
  modified_base 8: mod_base = OTHER; note = guanine phosphorothioate
  modified_base 9: mod_base = OTHER; note = beta-D-oxy LNA cytosine phosphorothioate
  modified_base 10: mod_base = OTHER; note = adenine phosphorothioate
  modified_base 11: mod_base = OTHER; note = beta-D-oxy LNA adenine phosphorothioate
  modified_base 12: mod_base = OTHER; note = beta-D-oxy LNA guanine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA guanine phosphorothioate
  modified_base 16: mod_base = OTHER; note = guanine phosphorothioate
  modified_base 17: mod_base = OTHER; note = beta-D-oxy LNA adenine phosphorothioate
  modified_base 18: mod_base = OTHER; note = beta-D-oxy LNA adenine
agatgaggcaagaaggaa

SEQ ID NO: 198 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = beta-D-oxy LNA adenine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = guanine phosphorothioate
  modified_base 6: mod_base = OTHER; note = beta-D-oxy LNA adenine phosphorothioate
  modified_base 7: mod_base = OTHER; note = beta-D-oxy LNA thymine phosphorothioate
  modified_base 8: mod_base = OTHER; note = guanine phosphorothioate
  modified_base 9: mod_base = OTHER; note = beta-D-oxy LNA adenine phosphorothioate
  modified_base 10: mod_base = OTHER; note = beta-D-oxy LNA guanine phosphorothioate
  modified_base 11: mod_base = OTHER; note = guanine phosphorothioate
  modified_base 12: mod_base = OTHER; note = beta-D-oxy LNA cytosine phosphorothioate
  modified_base 13: mod_base = OTHER; note = beta-D-oxy LNA adenine phosphorothioate
  modified_base 14: mod_base = OTHER; note = adenine phosphorothioate
  modified_base 15: mod_base = OTHER; note = beta-D-oxy LNA guanine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA guanine
cacagatgaggcaagaag

SEQ ID NO: 199 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA guanine phosphorothioate
  modified_base 3: mod_base = OTHER; note = thymine phosphorothioate
  modified_base 4: mod_base = OTHER; note = cytosine phosphorothioate
  modified_base 5: mod_base = OTHER; note = beta-D-oxy LNA adenine phosphorothioate
  modified_base 6: mod_base = OTHER; note = beta-D-oxy LNA cytosine phosphorothioate
  modified_base 7: mod_base = OTHER; note = adenine phosphorothioate
  modified_base 8: mod_base = OTHER; note = guanine phosphorothioate
  modified_base 9: mod_base = OTHER; note = beta-D-oxy LNA adenine phosphorothioate
  modified_base 10: mod_base = OTHER; note = beta-D-oxy LNA thymine phosphorothioate
  modified_base 11: mod_base = OTHER; note = guanine phosphorothioate
  modified_base 12: mod_base = OTHER; note = adenine phosphorothioate
  modified_base 13: mod_base = OTHER; note = beta-D-oxy LNA guanine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA guanine
agtcacagatgaggcaag

SEQ ID NO: 200 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = guanine phosphorothioate
  modified_base 3: mod_base = OTHER; note = thymine phosphorothioate
  modified_base 4: mod_base = OTHER; note = beta-D-oxy LNA adenine phosphorothioate
  modified_base 5: mod_base = OTHER; note = beta-D-oxy LNA guanine phosphorothioate
  modified_base 6: mod_base = OTHER; note = thymine phosphorothioate
  modified_base 7: mod_base = OTHER; note = cytosine phosphorothioate
  modified_base 8: mod_base = OTHER; note = beta-D-oxy LNA adenine phosphorothioate
  modified_base 9: mod_base = OTHER; note = beta-D-oxy LNA cytosine phosphorothioate
  modified_base 10: mod_base = OTHER; note = adenine phosphorothioate
  modified_base 11: mod_base = OTHER; note = guanine phosphorothioate
  modified_base 12: mod_base = OTHER; note = beta-D-oxy LNA adenine phosphorothioate
  modified_base 13: mod_base = OTHER; note = beta-D-oxy LNA thymine phosphorothioate
  modified_base 14: mod_base = OTHER; note = guanine phosphorothioate
  modified_base 15: mod_base = OTHER; note = beta-D-oxy LNA adenine phosphorothioate
  modified_base 16: mod_base = OTHER; note = guanine phosphorothioate
  modified_base 17: mod_base = OTHER; note = beta-D-oxy LNA guanine phosphorothioate
  modified_base 18: mod_base = OTHER; note = beta-D-oxy LNA cytosine
ggtagtcacagatgaggc

SEQ ID NO: 201 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = beta-D-oxy LNA adenine phosphorothioate
  modified_base 3: mod_base = OTHER; note = guanine phosphorothioate
  modified_base 4: mod_base = OTHER; note = guanine phosphorothioate
  modified_base 5: mod_base = OTHER; note = beta-D-oxy LNA guanine phosphorothioate
  modified_base 6: mod_base = OTHER; note = beta-D-oxy LNA thymine phosphorothioate
  modified_base 7: mod_base = OTHER; note = adenine phosphorothioate
  modified_base 8: mod_base = OTHER; note = beta-D-oxy LNA guanine phosphorothioate
  modified_base 9: mod_base = OTHER; note = thymine phosphorothioate
  modified_base 10: mod_base = OTHER; note = cytosine phosphorothioate
  modified_base 11: mod_base = OTHER; note = beta-D-oxy LNA adenine phosphorothioate
  modified_base 12: mod_base = OTHER; note = cytosine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = adenine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA adenine
gagggtagtcacagatga

SEQ ID NO: 202 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA thymine phosphorothioate
  modified_base 4: mod_base = OTHER; note = guanine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = guanine phosphorothioate
  modified_base 8: mod_base = OTHER; note = beta-D-oxy LNA guanine phosphorothioate
  modified_base 9: mod_base = OTHER; note = beta-D-oxy LNA thymine phosphorothioate
  modified_base 10: mod_base = OTHER; note = adenine phosphorothioate
  modified_base 11: mod_base = OTHER; note = beta-D-oxy LNA guanine phosphorothioate
  modified_base 12: mod_base = OTHER; note = thymine phosphorothioate
  modified_base 13: mod_base = OTHER; note = cytosine phosphorothioate
  modified_base 14: mod_base = OTHER; note = beta-D-oxy LNA adenine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA adenine
cctgagggtagtcacaga

SEQ ID NO: 203 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = thymine phosphorothioate
  modified_base 3: mod_base = OTHER; note = beta-D-oxy LNA guanine phosphorothioate
  modified_base 4: mod_base = OTHER; note = cytosine phosphorothioate
  modified_base 5: mod_base = OTHER; note = cytosine phosphorothioate
  modified_base 6: mod_base = OTHER; note = beta-D-oxy LNA thymine phosphorothioate
  modified_base 7: mod_base = OTHER; note = guanine phosphorothioate
  modified_base 8: mod_base = OTHER; note = adenine phosphorothioate
  modified_base 9: mod_base = OTHER; note = beta-D-oxy LNA guanine phosphorothioate
  modified_base 10: mod_base = OTHER; note = guanine phosphorothioate
  modified_base 11: mod_base = OTHER; note = beta-D-oxy LNA guanine phosphorothioate
  modified_base 12: mod_base = OTHER; note = thymine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = thymine phosphorothioate
  modified_base 16: mod_base = OTHER; note = cytosine phosphorothioate
  modified_base 17: mod_base = OTHER; note = beta-D-oxy LNA adenine phosphorothioate
  modified_base 18: mod_base = OTHER; note = beta-D-oxy LNA cytosine
gtgcctgagggtagtcac

SEQ ID NO: 204 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA thymine phosphorothioate
  modified_base 3: mod_base = OTHER; note = thymine phosphorothioate
  modified_base 4: mod_base = OTHER; note = beta-D-oxy LNA guanine phosphorothioate
  modified_base 5: mod_base = OTHER; note = thymine phosphorothioate
  modified_base 6: mod_base = OTHER; note = guanine phosphorothioate
  modified_base 7: mod_base = OTHER; note = beta-D-oxy LNA cytosine phosphorothioate
  modified_base 8: mod_base = OTHER; note = cytosine phosphorothioate
  modified_base 9: mod_base = OTHER; note = beta-D-oxy LNA thymine phosphorothioate
  modified_base 10: mod_base = OTHER; note = guanine phosphorothioate
  modified_base 11: mod_base = OTHER; note = adenine phosphorothioate
  modified_base 12: mod_base = OTHER; note = beta-D-oxy LNA guanine phosphorothioate
  modified_base 13: mod_base = OTHER; note = guanine phosphorothioate
  modified_base 14: mod_base = OTHER; note = beta-D-oxy LNA guanine phosphorothioate
  modified_base 15: mod_base = OTHER; note = thymine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA thymine
attgtgcctgagggtagt

SEQ ID NO: 205 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = thymine phosphorothioate
  modified_base 3: mod_base = OTHER; note = beta-D-oxy LNA adenine phosphorothioate
  modified_base 4: mod_base = OTHER; note = beta-D-oxy LNA adenine phosphorothioate
  modified_base 5: mod_base = OTHER; note = thymine phosphorothioate
  modified_base 6: mod_base = OTHER; note = thymine phosphorothioate
  modified_base 7: mod_base = OTHER; note = beta-D-oxy LNA guanine phosphorothioate
  modified_base 8: mod_base = OTHER; note = beta-D-oxy LNA thymine phosphorothioate
  modified_base 9: mod_base = OTHER; note = guanine phosphorothioate
  modified_base 10: mod_base = OTHER; note = cytosine phosphorothioate
  modified_base 11: mod_base = OTHER; note = beta-D-oxy LNA cytosine phosphorothioate
  modified_base 12: mod_base = OTHER; note = beta-D-oxy LNA thymine phosphorothioate
  modified_base 13: mod_base = OTHER; note = guanine phosphorothioate
  modified_base 14: mod_base = OTHER; note = adenine phosphorothioate
  modified_base 15: mod_base = OTHER; note = beta-D-oxy LNA guanine phosphorothioate
  modified_base 16: mod_base = OTHER; note = guanine phosphorothioate
  modified_base 17: mod_base = OTHER; note = beta-D-oxy LNA guanine phosphorothioate
  modified_base 18: mod_base = OTHER; note = beta-D-oxy LNA thymine
gtaattgtgcctgagggt

SEQ ID NO: 206 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = adenine phosphorothioate
  modified_base 3: mod_base = OTHER; note = guanine phosphorothioate
  modified_base 4: mod_base = OTHER; note = beta-D-oxy LNA guanine phosphorothioate
  modified_base 5: mod_base = OTHER; note = beta-D-oxy LNA thymine phosphorothioate
  modified_base 6: mod_base = OTHER; note = adenine phosphorothioate
  modified_base 7: mod_base = OTHER; note = beta-D-oxy LNA adenine phosphorothioate
  modified_base 8: mod_base = OTHER; note = beta-D-oxy LNA thymine phosphorothioate
  modified_base 9: mod_base = OTHER; note = thymine phosphorothioate
  modified_base 10: mod_base = OTHER; note = guanine phosphorothioate
  modified_base 11: mod_base = OTHER; note = beta-D-oxy LNA thymine phosphorothioate
  modified_base 12: mod_base = OTHER; note = guanine phosphorothioate
  modified_base 13: mod_base = OTHER; note = cytosine phosphorothioate
  modified_base 14: mod_base = OTHER; note = beta-D-oxy LNA cytosine phosphorothioate
  modified_base 15: mod_base = OTHER; note = beta-D-oxy LNA thymine phosphorothioate
  modified_base 16: mod_base = OTHER; note = guanine phosphorothioate
  modified_base 17: mod_base = OTHER; note = beta-D-oxy LNA adenine phosphorothioate
  modified_base 18: mod_base = OTHER; note = beta-D-oxy LNA guanine
caggtaattgtgcctgag

SEQ ID NO: 207 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA cytosine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = guanine phosphorothioate
  modified_base 8: mod_base = OTHER; note = thymine phosphorothioate
  modified_base 9: mod_base = OTHER; note = beta-D-oxy LNA adenine phosphorothioate
  modified_base 10: mod_base = OTHER; note = beta-D-oxy LNA adenine phosphorothioate
  modified_base 11: mod_base = OTHER; note = thymine phosphorothioate
  modified_base 12: mod_base = OTHER; note = thymine phosphorothioate
  modified_base 13: mod_base = OTHER; note = beta-D-oxy LNA guanine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA thymine
tcccaggtaattgtgcct

SEQ ID NO: 208 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = beta-D-oxy LNA thymine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = cytosine phosphorothioate
  modified_base 6: mod_base = OTHER; note = cytosine phosphorothioate
  modified_base 7: mod_base = OTHER; note = beta-D-oxy LNA cytosine phosphorothioate
  modified_base 8: mod_base = OTHER; note = adenine phosphorothioate
  modified_base 9: mod_base = OTHER; note = guanine phosphorothioate
  modified_base 10: mod_base = OTHER; note = beta-D-oxy LNA guanine phosphorothioate
  modified_base 11: mod_base = OTHER; note = beta-D-oxy LNA thymine phosphorothioate
  modified_base 12: mod_base = OTHER; note = adenine phosphorothioate
  modified_base 13: mod_base = OTHER; note = beta-D-oxy LNA adenine phosphorothioate
  modified_base 14: mod_base = OTHER; note = beta-D-oxy LNA thymine phosphorothioate
  modified_base 15: mod_base = OTHER; note = thymine phosphorothioate
  modified_base 16: mod_base = OTHER; note = guanine phosphorothioate
  modified_base 17: mod_base = OTHER; note = beta-D-oxy LNA thymine phosphorothioate
  modified_base 18: mod_base = OTHER; note = beta-D-oxy LNA guanine
ttctcccaggtaattgtg

SEQ ID NO: 209 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = guanine phosphorothioate
  modified_base 3: mod_base = OTHER; note = beta-D-oxy LNA guanine phosphorothioate
  modified_base 4: mod_base = OTHER; note = beta-D-oxy LNA thymine phosphorothioate
  modified_base 5: mod_base = OTHER; note = thymine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = cytosine phosphorothioate
  modified_base 9: mod_base = OTHER; note = cytosine phosphorothioate
  modified_base 10: mod_base = OTHER; note = beta-D-oxy LNA cytosine phosphorothioate
  modified_base 11: mod_base = OTHER; note = adenine phosphorothioate
  modified_base 12: mod_base = OTHER; note = guanine phosphorothioate
  modified_base 13: mod_base = OTHER; note = beta-D-oxy LNA guanine phosphorothioate
  modified_base 14: mod_base = OTHER; note = beta-D-oxy LNA thymine phosphorothioate
  modified_base 15: mod_base = OTHER; note = adenine phosphorothioate
  modified_base 16: mod_base = OTHER; note = adenine phosphorothioate
  modified_base 17: mod_base = OTHER; note = beta-D-oxy LNA thymine phosphorothioate
  modified_base 18: mod_base = OTHER; note = beta-D-oxy LNA thymine
aggttctcccaggtaatt

SEQ ID NO: 210 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = guanine phosphorothioate
  modified_base 6: mod_base = OTHER; note = guanine phosphorothioate
  modified_base 7: mod_base = OTHER; note = beta-D-oxy LNA thymine phosphorothioate
  modified_base 8: mod_base = OTHER; note = beta-D-oxy LNA thymine phosphorothioate
  modified_base 9: mod_base = OTHER; note = cytosine phosphorothioate
  modified_base 10: mod_base = OTHER; note = thymine phosphorothioate
  modified_base 11: mod_base = OTHER; note = beta-D-oxy LNA cytosine phosphorothioate
  modified_base 12: mod_base = OTHER; note = cytosine phosphorothioate
  modified_base 13: mod_base = OTHER; note = cytosine phosphorothioate
  modified_base 14: mod_base = OTHER; note = beta-D-oxy LNA adenine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA thymine phosphorothioate
  modified_base 18: mod_base = OTHER; note = beta-D-oxy LNA adenine
gccaggttctcccaggta

SEQ ID NO: 211 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = cytosine phosphorothioate
  modified_base 3: mod_base = OTHER; note = adenine phosphorothioate
  modified_base 4: mod_base = OTHER; note = beta-D-oxy LNA guanine phosphorothioate
  modified_base 5: mod_base = OTHER; note = cytosine phosphorothioate
  modified_base 6: mod_base = OTHER; note = cytosine phosphorothioate
  modified_base 7: mod_base = OTHER; note = beta-D-oxy LNA adenine phosphorothioate
  modified_base 8: mod_base = OTHER; note = guanine phosphorothioate
  modified_base 9: mod_base = OTHER; note = guanine phosphorothioate
  modified_base 10: mod_base = OTHER; note = beta-D-oxy LNA thymine phosphorothioate
  modified_base 11: mod_base = OTHER; note = beta-D-oxy LNA thymine phosphorothioate
  modified_base 12: mod_base = OTHER; note = cytosine phosphorothioate
  modified_base 13: mod_base = OTHER; note = thymine phosphorothioate
  modified_base 14: mod_base = OTHER; note = beta-D-oxy LNA cytosine phosphorothioate
  modified_base 15: mod_base = OTHER; note = cytosine phosphorothioate
  modified_base 16: mod_base = OTHER; note = cytosine phosphorothioate
  modified_base 17: mod_base = OTHER; note = beta-D-oxy LNA adenine phosphorothioate
  modified_base 18: mod_base = OTHER; note = beta-D-oxy LNA guanine
gcagccaggttctcccag

SEQ ID NO: 212 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = cytosine phosphorothioate
  modified_base 3: mod_base = OTHER; note = adenine phosphorothioate
  modified_base 4: mod_base = OTHER; note = beta-D-oxy LNA guanine phosphorothioate
  modified_base 5: mod_base = OTHER; note = cytosine phosphorothioate
  modified_base 6: mod_base = OTHER; note = adenine phosphorothioate
  modified_base 7: mod_base = OTHER; note = beta-D-oxy LNA guanine phosphorothioate
  modified_base 8: mod_base = OTHER; note = cytosine phosphorothioate
  modified_base 9: mod_base = OTHER; note = cytosine phosphorothioate
  modified_base 10: mod_base = OTHER; note = beta-D-oxy LNA adenine phosphorothioate
  modified_base 11: mod_base = OTHER; note = guanine phosphorothioate
  modified_base 12: mod_base = OTHER; note = guanine phosphorothioate
  modified_base 13: mod_base = OTHER; note = beta-D-oxy LNA thymine phosphorothioate
  modified_base 14: mod_base = OTHER; note = beta-D-oxy LNA thymine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA cytosine phosphorothioate
  modified_base 18: mod_base = OTHER; note = beta-D-oxy LNA cytosine
acagcagccaggttctcc

SEQ ID NO: 213 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA adenine phosphorothioate
  modified_base 3: mod_base = OTHER; note = thymine phosphorothioate
  modified_base 4: mod_base = OTHER; note = beta-D-oxy LNA adenine phosphorothioate
  modified_base 5: mod_base = OTHER; note = beta-D-oxy LNA cytosine phosphorothioate
  modified_base 6: mod_base = OTHER; note = adenine phosphorothioate
  modified_base 7: mod_base = OTHER; note = beta-D-oxy LNA guanine phosphorothioate
  modified_base 8: mod_base = OTHER; note = cytosine phosphorothioate
  modified_base 9: mod_base = OTHER; note = adenine phosphorothioate
  modified_base 10: mod_base = OTHER; note = beta-D-oxy LNA guanine phosphorothioate
  modified_base 11: mod_base = OTHER; note = cytosine phosphorothioate
  modified_base 12: mod_base = OTHER; note = cytosine phosphorothioate
  modified_base 13: mod_base = OTHER; note = beta-D-oxy LNA adenine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA cytosine
aatacagcagccaggttc

SEQ ID NO: 214 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = guanine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = adenine phosphorothioate
  modified_base 6: mod_base = OTHER; note = thymine phosphorothioate
  modified_base 7: mod_base = OTHER; note = beta-D-oxy LNA adenine phosphorothioate
  modified_base 8: mod_base = OTHER; note = cytosine phosphorothioate
  modified_base 9: mod_base = OTHER; note = adenine phosphorothioate
  modified_base 10: mod_base = OTHER; note = beta-D-oxy LNA guanine phosphorothioate
  modified_base 11: mod_base = OTHER; note = cytosine phosphorothioate
  modified_base 12: mod_base = OTHER; note = adenine phosphorothioate
  modified_base 13: mod_base = OTHER; note = beta-D-oxy LNA guanine phosphorothioate
  modified_base 14: mod_base = OTHER; note = beta-D-oxy LNA cytosine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA guanine
ggcaatacagcagccagg

SEQ ID NO: 215 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA adenine phosphorothioate
  modified_base 3: mod_base = OTHER; note = guanine phosphorothioate
  modified_base 4: mod_base = OTHER; note = guanine phosphorothioate
  modified_base 5: mod_base = OTHER; note = beta-D-oxy LNA guanine phosphorothioate
  modified_base 6: mod_base = OTHER; note = cytosine phosphorothioate
  modified_base 7: mod_base = OTHER; note = adenine phosphorothioate
  modified_base 8: mod_base = OTHER; note = beta-D-oxy LNA adenine phosphorothioate
  modified_base 9: mod_base = OTHER; note = beta-D-oxy LNA thymine phosphorothioate
  modified_base 10: mod_base = OTHER; note = adenine phosphorothioate
  modified_base 11: mod_base = OTHER; note = cytosine phosphorothioate
  modified_base 12: mod_base = OTHER; note = beta-D-oxy LNA adenine phosphorothioate
  modified_base 13: mod_base = OTHER; note = beta-D-oxy LNA guanine phosphorothioate
  modified_base 14: mod_base = OTHER; note = cytosine phosphorothioate
  modified_base 15: mod_base = OTHER; note = beta-D-oxy LNA adenine phosphorothioate
  modified_base 16: mod_base = OTHER; note = guanine phosphorothioate
  modified_base 17: mod_base = OTHER; note = beta-D-oxy LNA cytosine phosphorothioate
  modified_base 18: mod_base = OTHER; note = beta-D-oxy LNA cytosine
aagggcaatacagcagcc

SEQ ID NO: 216 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = beta-D-oxy LNA cytosine phosphorothioate
  modified_base 3: mod_base = OTHER; note = adenine phosphorothioate
  modified_base 4: mod_base = OTHER; note = adenine phosphorothioate
  modified_base 5: mod_base = OTHER; note = beta-D-oxy LNA adenine phosphorothioate
  modified_base 6: mod_base = OTHER; note = guanine phosphorothioate
  modified_base 7: mod_base = OTHER; note = guanine phosphorothioate
  modified_base 8: mod_base = OTHER; note = beta-D-oxy LNA guanine phosphorothioate
  modified_base 9: mod_base = OTHER; note = cytosine phosphorothioate
  modified_base 10: mod_base = OTHER; note = adenine phosphorothioate
  modified_base 11: mod_base = OTHER; note = beta-D-oxy LNA adenine phosphorothioate
  modified_base 12: mod_base = OTHER; note = beta-D-oxy LNA thymine phosphorothioate
  modified_base 13: mod_base = OTHER; note = adenine phosphorothioate
  modified_base 14: mod_base = OTHER; note = beta-D-oxy LNA cytosine phosphorothioate
  modified_base 15: mod_base = OTHER; note = beta-D-oxy LNA adenine phosphorothioate
  modified_base 16: mod_base = OTHER; note = guanine phosphorothioate
  modified_base 17: mod_base = OTHER; note = beta-D-oxy LNA cytosine phosphorothioate
  modified_base 18: mod_base = OTHER; note = beta-D-oxy LNA adenine
ccaaagggcaatacagca

SEQ ID NO: 217 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA thymine phosphorothioate
  modified_base 4: mod_base = OTHER; note = cytosine phosphorothioate
  modified_base 5: mod_base = OTHER; note = cytosine phosphorothioate
  modified_base 6: mod_base = OTHER; note = beta-D-oxy LNA adenine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = beta-D-oxy LNA guanine phosphorothioate
  modified_base 10: mod_base = OTHER; note = guanine phosphorothioate
  modified_base 11: mod_base = OTHER; note = beta-D-oxy LNA guanine phosphorothioate
  modified_base 12: mod_base = OTHER; note = beta-D-oxy LNA cytosine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA thymine phosphorothioate
  modified_base 16: mod_base = OTHER; note = adenine phosphorothioate
  modified_base 17: mod_base = OTHER; note = beta-D-oxy LNA cytosine phosphorothioate
  modified_base 18: mod_base = OTHER; note = beta-D-oxy LNA adenine
cctccaaagggcaataca

SEQ ID NO: 218 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = thymine phosphorothioate
  modified_base 3: mod_base = OTHER; note = beta-D-oxy LNA guanine phosphorothioate
  modified_base 4: mod_base = OTHER; note = cytosine phosphorothioate
  modified_base 5: mod_base = OTHER; note = beta-D-oxy LNA cytosine phosphorothioate
  modified_base 6: mod_base = OTHER; note = beta-D-oxy LNA thymine phosphorothioate
  modified_base 7: mod_base = OTHER; note = cytosine phosphorothioate
  modified_base 8: mod_base = OTHER; note = beta-D-oxy LNA cytosine phosphorothioate
  modified_base 9: mod_base = OTHER; note = adenine phosphorothioate
  modified_base 10: mod_base = OTHER; note = beta-D-oxy LNA adenine phosphorothioate
  modified_base 11: mod_base = OTHER; note = adenine phosphorothioate
  modified_base 12: mod_base = OTHER; note = guanine phosphorothioate
  modified_base 13: mod_base = OTHER; note = beta-D-oxy LNA guanine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA thymine
gtgcctccaaagggcaat

SEQ ID NO: 219 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = beta-D-oxy LNA thymine phosphorothioate
  modified_base 4: mod_base = OTHER; note = guanine phosphorothioate
  modified_base 5: mod_base = OTHER; note = beta-D-oxy LNA thymine phosphorothioate
  modified_base 6: mod_base = OTHER; note = guanine phosphorothioate
  modified_base 7: mod_base = OTHER; note = cytosine phosphorothioate
  modified_base 8: mod_base = OTHER; note = beta-D-oxy LNA cytosine phosphorothioate
  modified_base 9: mod_base = OTHER; note = beta-D-oxy LNA thymine phosphorothioate
  modified_base 10: mod_base = OTHER; note = cytosine phosphorothioate
  modified_base 11: mod_base = OTHER; note = cytosine phosphorothioate
  modified_base 12: mod_base = OTHER; note = beta-D-oxy LNA adenine phosphorothioate
  modified_base 13: mod_base = OTHER; note = adenine phosphorothioate
  modified_base 14: mod_base = OTHER; note = adenine phosphorothioate
  modified_base 15: mod_base = OTHER; note = beta-D-oxy LNA guanine phosphorothioate
  modified_base 16: mod_base = OTHER; note = guanine phosphorothioate
  modified_base 17: mod_base = OTHER; note = beta-D-oxy LNA guanine phosphorothioate
  modified_base 18: mod_base = OTHER; note = beta-D-oxy LNA cytosine
aatgtgcctccaaagggc

SEQ ID NO: 220 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = beta-D-oxy LNA adenine phosphorothioate
  modified_base 4: mod_base = OTHER; note = adenine phosphorothioate
  modified_base 5: mod_base = OTHER; note = beta-D-oxy LNA adenine phosphorothioate
  modified_base 6: mod_base = OTHER; note = beta-D-oxy LNA thymine phosphorothioate
  modified_base 7: mod_base = OTHER; note = guanine phosphorothioate
  modified_base 8: mod_base = OTHER; note = beta-D-oxy LNA thymine phosphorothioate
  modified_base 9: mod_base = OTHER; note = guanine phosphorothioate
  modified_base 10: mod_base = OTHER; note = beta-D-oxy LNA cytosine phosphorothioate
  modified_base 11: mod_base = OTHER; note = beta-D-oxy LNA cytosine phosphorothioate
  modified_base 12: mod_base = OTHER; note = thymine phosphorothioate
  modified_base 13: mod_base = OTHER; note = beta-D-oxy LNA cytosine phosphorothioate
  modified_base 14: mod_base = OTHER; note = beta-D-oxy LNA cytosine phosphorothioate
  modified_base 15: mod_base = OTHER; note = adenine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA guanine
tcaaatgtgcctccaaag

SEQ ID NO: 221 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = guanine phosphorothioate
  modified_base 3: mod_base = OTHER; note = beta-D-oxy LNA guanine phosphorothioate
  modified_base 4: mod_base = OTHER; note = thymine phosphorothioate
  modified_base 5: mod_base = OTHER; note = cytosine phosphorothioate
  modified_base 6: mod_base = OTHER; note = beta-D-oxy LNA adenine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = thymine phosphorothioate
  modified_base 10: mod_base = OTHER; note = beta-D-oxy LNA guanine phosphorothioate
  modified_base 11: mod_base = OTHER; note = beta-D-oxy LNA thymine phosphorothioate
  modified_base 12: mod_base = OTHER; note = guanine phosphorothioate
  modified_base 13: mod_base = OTHER; note = cytosine phosphorothioate
  modified_base 14: mod_base = OTHER; note = beta-D-oxy LNA cytosine phosphorothioate
  modified_base 15: mod_base = OTHER; note = beta-D-oxy LNA thymine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA adenine
aggtcaaatgtgcctcca

SEQ ID NO: 222 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = cytosine phosphorothioate
  modified_base 3: mod_base = OTHER; note = beta-D-oxy LNA thymine phosphorothioate
  modified_base 4: mod_base = OTHER; note = beta-D-oxy LNA adenine phosphorothioate
  modified_base 5: mod_base = OTHER; note = guanine phosphorothioate
  modified_base 6: mod_base = OTHER; note = beta-D-oxy LNA guanine phosphorothioate
  modified_base 7: mod_base = OTHER; note = beta-D-oxy LNA thymine phosphorothioate
  modified_base 8: mod_base = OTHER; note = cytosine phosphorothioate
  modified_base 9: mod_base = OTHER; note = beta-D-oxy LNA adenine phosphorothioate
  modified_base 10: mod_base = OTHER; note = adenine phosphorothioate
  modified_base 11: mod_base = OTHER; note = beta-D-oxy LNA adenine phosphorothioate
  modified_base 12: mod_base = OTHER; note = beta-D-oxy LNA thymine phosphorothioate
  modified_base 13: mod_base = OTHER; note = guanine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA thymine
actaggtcaaatgtgcct

SEQ ID NO: 223 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = beta-D-oxy LNA guanine phosphorothioate
  modified_base 4: mod_base = OTHER; note = beta-D-oxy LNA adenine phosphorothioate
  modified_base 5: mod_base = OTHER; note = cytosine phosphorothioate
  modified_base 6: mod_base = OTHER; note = beta-D-oxy LNA thymine phosphorothioate
  modified_base 7: mod_base = OTHER; note = adenine phosphorothioate
  modified_base 8: mod_base = OTHER; note = beta-D-oxy LNA guanine phosphorothioate
  modified_base 9: mod_base = OTHER; note = guanine phosphorothioate
  modified_base 10: mod_base = OTHER; note = thymine phosphorothioate
  modified_base 11: mod_base = OTHER; note = beta-D-oxy LNA cytosine phosphorothioate
  modified_base 12: mod_base = OTHER; note = beta-D-oxy LNA adenine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA thymine phosphorothioate
  modified_base 16: mod_base = OTHER; note = guanine phosphorothioate
  modified_base 17: mod_base = OTHER; note = beta-D-oxy LNA thymine phosphorothioate
  modified_base 18: mod_base = OTHER; note = beta-D-oxy LNA guanine
tggactaggtcaaatgtg

SEQ ID NO: 224 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = beta-D-oxy LNA cytosine phosphorothioate
  modified_base 4: mod_base = OTHER; note = beta-D-oxy LNA thymine phosphorothioate
  modified_base 5: mod_base = OTHER; note = guanine phosphorothioate
  modified_base 6: mod_base = OTHER; note = beta-D-oxy LNA guanine phosphorothioate
  modified_base 7: mod_base = OTHER; note = beta-D-oxy LNA adenine phosphorothioate
  modified_base 8: mod_base = OTHER; note = cytosine phosphorothioate
  modified_base 9: mod_base = OTHER; note = beta-D-oxy LNA thymine phosphorothioate
  modified_base 10: mod_base = OTHER; note = adenine phosphorothioate
  modified_base 11: mod_base = OTHER; note = beta-D-oxy LNA guanine phosphorothioate
  modified_base 12: mod_base = OTHER; note = beta-D-oxy LNA guanine phosphorothioate
  modified_base 13: mod_base = OTHER; note = thymine phosphorothioate
  modified_base 14: mod_base = OTHER; note = beta-D-oxy LNA cytosine phosphorothioate
  modified_base 15: mod_base = OTHER; note = beta-D-oxy LNA adenine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA thymine
aactggactaggtcaaat

SEQ ID NO: 225 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = beta-D-oxy LNA thymine phosphorothioate
  modified_base 3: mod_base = OTHER; note = thymine phosphorothioate
  modified_base 4: mod_base = OTHER; note = beta-D-oxy LNA adenine phosphorothioate
  modified_base 5: mod_base = OTHER; note = adenine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = guanine phosphorothioate
  modified_base 9: mod_base = OTHER; note = beta-D-oxy LNA guanine phosphorothioate
  modified_base 10: mod_base = OTHER; note = beta-D-oxy LNA adenine phosphorothioate
  modified_base 11: mod_base = OTHER; note = cytosine phosphorothioate
  modified_base 12: mod_base = OTHER; note = beta-D-oxy LNA thymine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = beta-D-oxy LNA guanine phosphorothioate
  modified_base 16: mod_base = OTHER; note = thymine phosphorothioate
  modified_base 17: mod_base = OTHER; note = beta-D-oxy LNA cytosine phosphorothioate
  modified_base 18: mod_base = OTHER; note = beta-D-oxy LNA adenine
tttaactggactaggtca

SEQ ID NO: 226 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA thymine phosphorothioate
  modified_base 4: mod_base = OTHER; note = beta-D-oxy LNA thymine phosphorothioate
  modified_base 5: mod_base = OTHER; note = thymine phosphorothioate
  modified_base 6: mod_base = OTHER; note = thymine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = beta-D-oxy LNA cytosine phosphorothioate
  modified_base 10: mod_base = OTHER; note = beta-D-oxy LNA thymine phosphorothioate
  modified_base 11: mod_base = OTHER; note = guanine phosphorothioate
  modified_base 12: mod_base = OTHER; note = beta-D-oxy LNA guanine phosphorothioate
  modified_base 13: mod_base = OTHER; note = beta-D-oxy LNA adenine phosphorothioate
  modified_base 14: mod_base = OTHER; note = cytosine phosphorothioate
  modified_base 15: mod_base = OTHER; note = beta-D-oxy LNA thymine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA guanine
ccttttaactggactagg

SEQ ID NO: 227 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = beta-D-oxy LNA adenine phosphorothioate
  modified_base 3: mod_base = OTHER; note = adenine phosphorothioate
  modified_base 4: mod_base = OTHER; note = cytosine phosphorothioate
  modified_base 5: mod_base = OTHER; note = beta-D-oxy LNA cytosine phosphorothioate
  modified_base 6: mod_base = OTHER; note = beta-D-oxy LNA thymine phosphorothioate
  modified_base 7: mod_base = OTHER; note = thymine phosphorothioate
  modified_base 8: mod_base = OTHER; note = thymine phosphorothioate
  modified_base 9: mod_base = OTHER; note = beta-D-oxy LNA thymine phosphorothioate
  modified_base 10: mod_base = OTHER; note = beta-D-oxy LNA adenine phosphorothioate
  modified_base 11: mod_base = OTHER; note = adenine phosphorothioate
  modified_base 12: mod_base = OTHER; note = beta-D-oxy LNA cytosine phosphorothioate
  modified_base 13: mod_base = OTHER; note = beta-D-oxy LNA thymine phosphorothioate
  modified_base 14: mod_base = OTHER; note = guanine phosphorothioate
  modified_base 15: mod_base = OTHER; note = beta-D-oxy LNA guanine phosphorothioate
  modified_base 16: mod_base = OTHER; note = adenine phosphorothioate
  modified_base 17: mod_base = OTHER; note = beta-D-oxy LNA cytosine phosphorothioate
  modified_base 18: mod_base = OTHER; note = beta-D-oxy LNA thymine
gaaccttttaactggact

SEQ ID NO: 228 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = beta-D-oxy LNA thymine phosphorothioate
  modified_base 3: mod_base = OTHER; note = guanine phosphorothioate
  modified_base 4: mod_base = OTHER; note = beta-D-oxy LNA guanine phosphorothioate
  modified_base 5: mod_base = OTHER; note = beta-D-oxy LNA adenine phosphorothioate
  modified_base 6: mod_base = OTHER; note = adenine phosphorothioate
  modified_base 7: mod_base = OTHER; note = cytosine phosphorothioate
  modified_base 8: mod_base = OTHER; note = beta-D-oxy LNA cytosine phosphorothioate
  modified_base 9: mod_base = OTHER; note = beta-D-oxy LNA thymine phosphorothioate
  modified_base 10: mod_base = OTHER; note = thymine phosphorothioate
  modified_base 11: mod_base = OTHER; note = thymine phosphorothioate
  modified_base 12: mod_base = OTHER; note = beta-D-oxy LNA thymine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = beta-D-oxy LNA cytosine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA guanine
gtggaaccttttaactgg

SEQ ID NO: 229 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA guanine phosphorothioate
  modified_base 3: mod_base = OTHER; note = guanine phosphorothioate
  modified_base 4: mod_base = OTHER; note = beta-D-oxy LNA guanine phosphorothioate
  modified_base 5: mod_base = OTHER; note = beta-D-oxy LNA thymine phosphorothioate
  modified_base 6: mod_base = OTHER; note = guanine phosphorothioate
  modified_base 7: mod_base = OTHER; note = beta-D-oxy LNA guanine phosphorothioate
  modified_base 8: mod_base = OTHER; note = beta-D-oxy LNA adenine phosphorothioate
  modified_base 9: mod_base = OTHER; note = adenine phosphorothioate
  modified_base 10: mod_base = OTHER; note = cytosine phosphorothioate
  modified_base 11: mod_base = OTHER; note = beta-D-oxy LNA cytosine phosphorothioate
  modified_base 12: mod_base = OTHER; note = beta-D-oxy LNA thymine phosphorothioate
  modified_base 13: mod_base = OTHER; note = thymine phosphorothioate
  modified_base 14: mod_base = OTHER; note = beta-D-oxy LNA thymine phosphorothioate
  modified_base 15: mod_base = OTHER; note = thymine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA cytosine
agggtggaaccttttaac

SEQ ID NO: 230 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = beta-D-oxy LNA thymine phosphorothioate
  modified_base 3: mod_base = OTHER; note = cytosine phosphorothioate
  modified_base 4: mod_base = OTHER; note = adenine phosphorothioate
  modified_base 5: mod_base = OTHER; note = beta-D-oxy LNA guanine phosphorothioate
  modified_base 6: mod_base = OTHER; note = guanine phosphorothioate
  modified_base 7: mod_base = OTHER; note = beta-D-oxy LNA guanine phosphorothioate
  modified_base 8: mod_base = OTHER; note = beta-D-oxy LNA thymine phosphorothioate
  modified_base 9: mod_base = OTHER; note = guanine phosphorothioate
  modified_base 10: mod_base = OTHER; note = guanine phosphorothioate
  modified_base 11: mod_base = OTHER; note = beta-D-oxy LNA adenine phosphorothioate
  modified_base 12: mod_base = OTHER; note = beta-D-oxy LNA adenine phosphorothioate
  modified_base 13: mod_base = OTHER; note = cytosine phosphorothioate
  modified_base 14: mod_base = OTHER; note = beta-D-oxy LNA cytosine phosphorothioate
  modified_base 15: mod_base = OTHER; note = beta-D-oxy LNA thymine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA thymine
ttcagggtggaacctttt

SEQ ID NO: 231 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = thymine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = beta-D-oxy LNA thymine phosphorothioate
  modified_base 6: mod_base = OTHER; note = cytosine phosphorothioate
  modified_base 7: mod_base = OTHER; note = adenine phosphorothioate
  modified_base 8: mod_base = OTHER; note = beta-D-oxy LNA guanine phosphorothioate
  modified_base 9: mod_base = OTHER; note = guanine phosphorothioate
  modified_base 10: mod_base = OTHER; note = beta-D-oxy LNA guanine phosphorothioate
  modified_base 11: mod_base = OTHER; note = beta-D-oxy LNA thymine phosphorothioate
  modified_base 12: mod_base = OTHER; note = guanine phosphorothioate
  modified_base 13: mod_base = OTHER; note = guanine phosphorothioate
  modified_base 14: mod_base = OTHER; note = beta-D-oxy LNA adenine phosphorothioate
  modified_base 15: mod_base = OTHER; note = beta-D-oxy LNA adenine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA thymine
ttcttcagggtggaacct

SEQ ID NO: 232 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = thymine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = thymine phosphorothioate
  modified_base 9: mod_base = OTHER; note = beta-D-oxy LNA cytosine phosphorothioate
  modified_base 10: mod_base = OTHER; note = beta-D-oxy LNA adenine phosphorothioate
  modified_base 11: mod_base = OTHER; note = guanine phosphorothioate
  modified_base 12: mod_base = OTHER; note = guanine phosphorothioate
  modified_base 13: mod_base = OTHER; note = beta-D-oxy LNA guanine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA adenine phosphorothioate
  modified_base 18: mod_base = OTHER; note = beta-D-oxy LNA adenine
gccttcttcagggtggaa

SEQ ID NO: 233 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = guanine phosphorothioate
  modified_base 4: mod_base = OTHER; note = beta-D-oxy LNA guanine phosphorothioate
  modified_base 5: mod_base = OTHER; note = cytosine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = thymine phosphorothioate
  modified_base 9: mod_base = OTHER; note = cytosine phosphorothioate
  modified_base 10: mod_base = OTHER; note = beta-D-oxy LNA thymine phosphorothioate
  modified_base 11: mod_base = OTHER; note = thymine phosphorothioate
  modified_base 12: mod_base = OTHER; note = cytosine phosphorothioate
  modified_base 13: mod_base = OTHER; note = beta-D-oxy LNA adenine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA thymine phosphorothioate
  modified_base 18: mod_base = OTHER; note = beta-D-oxy LNA guanine
aaggccttcttcagggtg

SEQ ID NO: 234 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = beta-D-oxy LNA adenine phosphorothioate
  modified_base 4: mod_base = OTHER; note = adenine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = beta-D-oxy LNA guanine phosphorothioate
  modified_base 8: mod_base = OTHER; note = cytosine phosphorothioate
  modified_base 9: mod_base = OTHER; note = cytosine phosphorothioate
  modified_base 10: mod_base = OTHER; note = beta-D-oxy LNA thymine phosphorothioate
  modified_base 11: mod_base = OTHER; note = beta-D-oxy LNA thymine phosphorothioate
  modified_base 12: mod_base = OTHER; note = cytosine phosphorothioate
  modified_base 13: mod_base = OTHER; note = beta-D-oxy LNA thymine phosphorothioate
  modified_base 14: mod_base = OTHER; note = beta-D-oxy LNA thymine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA guanine
tcaaaggccttcttcagg

SEQ ID NO: 235 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = cytosine phosphorothioate
  modified_base 6: mod_base = OTHER; note = beta-D-oxy LNA adenine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = beta-D-oxy LNA guanine phosphorothioate
  modified_base 10: mod_base = OTHER; note = beta-D-oxy LNA guanine phosphorothioate
  modified_base 11: mod_base = OTHER; note = cytosine phosphorothioate
  modified_base 12: mod_base = OTHER; note = cytosine phosphorothioate
  modified_base 13: mod_base = OTHER; note = beta-D-oxy LNA thymine phosphorothioate
  modified_base 14: mod_base = OTHER; note = beta-D-oxy LNA thymine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA cytosine
tgctcaaaggccttcttc

SEQ ID NO: 236 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = thymine phosphorothioate
  modified_base 3: mod_base = OTHER; note = beta-D-oxy LNA adenine phosphorothioate
  modified_base 4: mod_base = OTHER; note = beta-D-oxy LNA thymine phosphorothioate
  modified_base 5: mod_base = OTHER; note = guanine phosphorothioate
  modified_base 6: mod_base = OTHER; note = beta-D-oxy LNA cytosine phosphorothioate
  modified_base 7: mod_base = OTHER; note = beta-D-oxy LNA thymine phosphorothioate
  modified_base 8: mod_base = OTHER; note = cytosine phosphorothioate
  modified_base 9: mod_base = OTHER; note = beta-D-oxy LNA adenine phosphorothioate
  modified_base 10: mod_base = OTHER; note = adenine phosphorothioate
  modified_base 11: mod_base = OTHER; note = adenine phosphorothioate
  modified_base 12: mod_base = OTHER; note = beta-D-oxy LNA guanine phosphorothioate
  modified_base 13: mod_base = OTHER; note = guanine phosphorothioate
  modified_base 14: mod_base = OTHER; note = beta-D-oxy LNA cytosine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA cytosine
ctatgctcaaaggccttc

SEQ ID NO: 237 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = cytosine phosphorothioate
  modified_base 3: mod_base = OTHER; note = beta-D-oxy LNA guanine phosphorothioate
  modified_base 4: mod_base = OTHER; note = beta-D-oxy LNA cytosine phosphorothioate
  modified_base 5: mod_base = OTHER; note = thymine phosphorothioate
  modified_base 6: mod_base = OTHER; note = adenine phosphorothioate
  modified_base 7: mod_base = OTHER; note = beta-D-oxy LNA thymine phosphorothioate
  modified_base 8: mod_base = OTHER; note = guanine phosphorothioate
  modified_base 9: mod_base = OTHER; note = cytosine phosphorothioate
  modified_base 10: mod_base = OTHER; note = beta-D-oxy LNA thymine phosphorothioate
  modified_base 11: mod_base = OTHER; note = cytosine phosphorothioate
  modified_base 12: mod_base = OTHER; note = beta-D-oxy LNA adenine phosphorothioate
  modified_base 13: mod_base = OTHER; note = adenine phosphorothioate
  modified_base 14: mod_base = OTHER; note = adenine phosphorothioate
  modified_base 15: mod_base = OTHER; note = beta-D-oxy LNA guanine phosphorothioate
  modified_base 16: mod_base = OTHER; note = guanine phosphorothioate
  modified_base 17: mod_base = OTHER; note = beta-D-oxy LNA cytosine phosphorothioate
  modified_base 18: mod_base = OTHER; note = beta-D-oxy LNA cytosine
acgctatgctcaaaggcc

SEQ ID NO: 238 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = beta-D-oxy LNA cytosine phosphorothioate
  modified_base 4: mod_base = OTHER; note = beta-D-oxy LNA adenine phosphorothioate
  modified_base 5: mod_base = OTHER; note = cytosine phosphorothioate
  modified_base 6: mod_base = OTHER; note = guanine phosphorothioate
  modified_base 7: mod_base = OTHER; note = beta-D-oxy LNA cytosine phosphorothioate
  modified_base 8: mod_base = OTHER; note = beta-D-oxy LNA thymine phosphorothioate
  modified_base 9: mod_base = OTHER; note = adenine phosphorothioate
  modified_base 10: mod_base = OTHER; note = beta-D-oxy LNA thymine phosphorothioate
  modified_base 11: mod_base = OTHER; note = guanine phosphorothioate
  modified_base 12: mod_base = OTHER; note = beta-D-oxy LNA cytosine phosphorothioate
  modified_base 13: mod_base = OTHER; note = beta-D-oxy LNA thymine phosphorothioate
  modified_base 14: mod_base = OTHER; note = cytosine phosphorothioate
  modified_base 15: mod_base = OTHER; note = beta-D-oxy LNA adenine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA guanine
tgcacgctatgctcaaag

SEQ ID NO: 239 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = guanine phosphorothioate
  modified_base 3: mod_base = OTHER; note = beta-D-oxy LNA guanine phosphorothioate
  modified_base 4: mod_base = OTHER; note = thymine phosphorothioate
  modified_base 5: mod_base = OTHER; note = beta-D-oxy LNA guanine phosphorothioate
  modified_base 6: mod_base = OTHER; note = cytosine phosphorothioate
  modified_base 7: mod_base = OTHER; note = beta-D-oxy LNA adenine phosphorothioate
  modified_base 8: mod_base = OTHER; note = cytosine phosphorothioate
  modified_base 9: mod_base = OTHER; note = guanine phosphorothioate
  modified_base 10: mod_base = OTHER; note = beta-D-oxy LNA cytosine phosphorothioate
  modified_base 11: mod_base = OTHER; note = beta-D-oxy LNA thymine phosphorothioate
  modified_base 12: mod_base = OTHER; note = adenine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA cytosine phosphorothioate
  modified_base 18: mod_base = OTHER; note = beta-D-oxy LNA adenine
cggtgcacgctatgctca

SEQ ID NO: 240 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = adenine phosphorothioate
  modified_base 3: mod_base = OTHER; note = beta-D-oxy LNA guanine phosphorothioate
  modified_base 4: mod_base = OTHER; note = cytosine phosphorothioate
  modified_base 5: mod_base = OTHER; note = beta-D-oxy LNA guanine phosphorothioate
  modified_base 6: mod_base = OTHER; note = guanine phosphorothioate
  modified_base 7: mod_base = OTHER; note = thymine phosphorothioate
  modified_base 8: mod_base = OTHER; note = beta-D-oxy LNA guanine phosphorothioate
  modified_base 9: mod_base = OTHER; note = cytosine phosphorothioate
  modified_base 10: mod_base = OTHER; note = beta-D-oxy LNA adenine phosphorothioate
  modified_base 11: mod_base = OTHER; note = beta-D-oxy LNA cytosine phosphorothioate
  modified_base 12: mod_base = OTHER; note = guanine phosphorothioate
  modified_base 13: mod_base = OTHER; note = cytosine phosphorothioate
  modified_base 14: mod_base = OTHER; note = beta-D-oxy LNA thymine phosphorothioate
  modified_base 15: mod_base = OTHER; note = adenine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA cytosine
tagcggtgcacgctatgc

SEQ ID NO: 241 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA guanine phosphorothioate
  modified_base 4: mod_base = OTHER; note = thymine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = cytosine phosphorothioate
  modified_base 8: mod_base = OTHER; note = guanine phosphorothioate
  modified_base 9: mod_base = OTHER; note = beta-D-oxy LNA guanine phosphorothioate
  modified_base 10: mod_base = OTHER; note = thymine phosphorothioate
  modified_base 11: mod_base = OTHER; note = beta-D-oxy LNA guanine phosphorothioate
  modified_base 12: mod_base = OTHER; note = cytosine phosphorothioate
  modified_base 13: mod_base = OTHER; note = adenine phosphorothioate
  modified_base 14: mod_base = OTHER; note = beta-D-oxy LNA cytosine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA thymine phosphorothioate
  modified_base 18: mod_base = OTHER; note = beta-D-oxy LNA adenine
ccgtagcggtgcacgcta

SEQ ID NO: 242 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = guanine phosphorothioate
  modified_base 4: mod_base = OTHER; note = beta-D-oxy LNA cytosine phosphorothioate
  modified_base 5: mod_base = OTHER; note = cytosine phosphorothioate
  modified_base 6: mod_base = OTHER; note = beta-D-oxy LNA guanine phosphorothioate
  modified_base 7: mod_base = OTHER; note = thymine phosphorothioate
  modified_base 8: mod_base = OTHER; note = beta-D-oxy LNA adenine phosphorothioate
  modified_base 9: mod_base = OTHER; note = guanine phosphorothioate
  modified_base 10: mod_base = OTHER; note = cytosine phosphorothioate
  modified_base 11: mod_base = OTHER; note = beta-D-oxy LNA guanine phosphorothioate
  modified_base 12: mod_base = OTHER; note = guanine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA cytosine phosphorothioate
  modified_base 18: mod_base = OTHER; note = beta-D-oxy LNA guanine
tggccgtagcggtgcacg

SEQ ID NO: 243 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = adenine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = guanine phosphorothioate
  modified_base 6: mod_base = OTHER; note = beta-D-oxy LNA guanine phosphorothioate
  modified_base 7: mod_base = OTHER; note = cytosine phosphorothioate
  modified_base 8: mod_base = OTHER; note = cytosine phosphorothioate
  modified_base 9: mod_base = OTHER; note = beta-D-oxy LNA guanine phosphorothioate
  modified_base 10: mod_base = OTHER; note = thymine phosphorothioate
  modified_base 11: mod_base = OTHER; note = adenine phosphorothioate
  modified_base 12: mod_base = OTHER; note = beta-D-oxy LNA guanine phosphorothioate
  modified_base 13: mod_base = OTHER; note = cytosine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA cytosine
gactggccgtagcggtgc

SEQ ID NO: 244 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = thymine phosphorothioate
  modified_base 3: mod_base = OTHER; note = guanine phosphorothioate
  modified_base 4: mod_base = OTHER; note = beta-D-oxy LNA guanine phosphorothioate
  modified_base 5: mod_base = OTHER; note = adenine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = guanine phosphorothioate
  modified_base 9: mod_base = OTHER; note = beta-D-oxy LNA guanine phosphorothioate
  modified_base 10: mod_base = OTHER; note = cytosine phosphorothioate
  modified_base 11: mod_base = OTHER; note = cytosine phosphorothioate
  modified_base 12: mod_base = OTHER; note = beta-D-oxy LNA guanine phosphorothioate
  modified_base 13: mod_base = OTHER; note = thymine phosphorothioate
  modified_base 14: mod_base = OTHER; note = beta-D-oxy LNA adenine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA guanine phosphorothioate
  modified_base 18: mod_base = OTHER; note = beta-D-oxy LNA guanine
gtggactggccgtagcgg

SEQ ID NO: 245 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = adenine phosphorothioate
  modified_base 4: mod_base = OTHER; note = beta-D-oxy LNA guanine phosphorothioate
  modified_base 5: mod_base = OTHER; note = thymine phosphorothioate
  modified_base 6: mod_base = OTHER; note = guanine phosphorothioate
  modified_base 7: mod_base = OTHER; note = beta-D-oxy LNA guanine phosphorothioate
  modified_base 8: mod_base = OTHER; note = adenine phosphorothioate
  modified_base 9: mod_base = OTHER; note = cytosine phosphorothioate
  modified_base 10: mod_base = OTHER; note = beta-D-oxy LNA thymine phosphorothioate
  modified_base 11: mod_base = OTHER; note = guanine phosphorothioate
  modified_base 12: mod_base = OTHER; note = beta-D-oxy LNA guanine phosphorothioate
  modified_base 13: mod_base = OTHER; note = cytosine phosphorothioate
  modified_base 14: mod_base = OTHER; note = cytosine phosphorothioate
  modified_base 15: mod_base = OTHER; note = beta-D-oxy LNA guanine phosphorothioate
  modified_base 16: mod_base = OTHER; note = thymine phosphorothioate
  modified_base 17: mod_base = OTHER; note = beta-D-oxy LNA adenine phosphorothioate
  modified_base 18: mod_base = OTHER; note = beta-D-oxy LNA guanine
ccagtggactggccgtag

SEQ ID NO: 246 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = beta-D-oxy LNA thymine phosphorothioate
  modified_base 4: mod_base = OTHER; note = cytosine phosphorothioate
  modified_base 5: mod_base = OTHER; note = cytosine phosphorothioate
  modified_base 6: mod_base = OTHER; note = beta-D-oxy LNA adenine phosphorothioate
  modified_base 7: mod_base = OTHER; note = guanine phosphorothioate
  modified_base 8: mod_base = OTHER; note = thymine phosphorothioate
  modified_base 9: mod_base = OTHER; note = beta-D-oxy LNA guanine phosphorothioate
  modified_base 10: mod_base = OTHER; note = guanine phosphorothioate
  modified_base 11: mod_base = OTHER; note = beta-D-oxy LNA adenine phosphorothioate
  modified_base 12: mod_base = OTHER; note = cytosine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA guanine
tctccagtggactggccg

SEQ ID NO: 247 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = adenine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = cytosine phosphorothioate
  modified_base 6: mod_base = OTHER; note = beta-D-oxy LNA thymine phosphorothioate
  modified_base 7: mod_base = OTHER; note = cytosine phosphorothioate
  modified_base 8: mod_base = OTHER; note = cytosine phosphorothioate
  modified_base 9: mod_base = OTHER; note = beta-D-oxy LNA adenine phosphorothioate
  modified_base 10: mod_base = OTHER; note = beta-D-oxy LNA guanine phosphorothioate
  modified_base 11: mod_base = OTHER; note = thymine phosphorothioate
  modified_base 12: mod_base = OTHER; note = guanine phosphorothioate
  modified_base 13: mod_base = OTHER; note = beta-D-oxy LNA guanine phosphorothioate
  modified_base 14: mod_base = OTHER; note = beta-D-oxy LNA adenine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA guanine
aactctccagtggactgg

SEQ ID NO: 248 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA guanine phosphorothioate
  modified_base 3: mod_base = OTHER; note = guanine phosphorothioate
  modified_base 4: mod_base = OTHER; note = beta-D-oxy LNA adenine phosphorothioate
  modified_base 5: mod_base = OTHER; note = adenine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = cytosine phosphorothioate
  modified_base 9: mod_base = OTHER; note = thymine phosphorothioate
  modified_base 10: mod_base = OTHER; note = beta-D-oxy LNA cytosine phosphorothioate
  modified_base 11: mod_base = OTHER; note = cytosine phosphorothioate
  modified_base 12: mod_base = OTHER; note = adenine phosphorothioate
  modified_base 13: mod_base = OTHER; note = beta-D-oxy LNA guanine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA adenine phosphorothioate
  modified_base 18: mod_base = OTHER; note = beta-D-oxy LNA cytosine
aggaactctccagtggac

SEQ ID NO: 249 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = guanine phosphorothioate
  modified_base 6: mod_base = OTHER; note = guanine phosphorothioate
  modified_base 7: mod_base = OTHER; note = beta-D-oxy LNA adenine phosphorothioate
  modified_base 8: mod_base = OTHER; note = beta-D-oxy LNA adenine phosphorothioate
  modified_base 9: mod_base = OTHER; note = cytosine phosphorothioate
  modified_base 10: mod_base = OTHER; note = thymine phosphorothioate
  modified_base 11: mod_base = OTHER; note = beta-D-oxy LNA cytosine phosphorothioate
  modified_base 12: mod_base = OTHER; note = beta-D-oxy LNA thymine phosphorothioate
  modified_base 13: mod_base = OTHER; note = cytosine phosphorothioate
  modified_base 14: mod_base = OTHER; note = cytosine phosphorothioate
  modified_base 15: mod_base = OTHER; note = beta-D-oxy LNA adenine phosphorothioate
  modified_base 16: mod_base = OTHER; note = guanine phosphorothioate
  modified_base 17: mod_base = OTHER; note = beta-D-oxy LNA thymine phosphorothioate
  modified_base 18: mod_base = OTHER; note = beta-D-oxy LNA guanine
tgcaggaactctccagtg

SEQ ID NO: 250 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = guanine phosphorothioate
  modified_base 6: mod_base = OTHER; note = cytosine phosphorothioate
  modified_base 7: mod_base = OTHER; note = beta-D-oxy LNA adenine phosphorothioate
  modified_base 8: mod_base = OTHER; note = beta-D-oxy LNA guanine phosphorothioate
  modified_base 9: mod_base = OTHER; note = guanine phosphorothioate
  modified_base 10: mod_base = OTHER; note = beta-D-oxy LNA adenine phosphorothioate
  modified_base 11: mod_base = OTHER; note = adenine phosphorothioate
  modified_base 12: mod_base = OTHER; note = cytosine phosphorothioate
  modified_base 13: mod_base = OTHER; note = beta-D-oxy LNA thymine phosphorothioate
  modified_base 14: mod_base = OTHER; note = cytosine phosphorothioate
  modified_base 15: mod_base = OTHER; note = beta-D-oxy LNA thymine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA adenine
acctgcaggaactctcca

SEQ ID NO: 251 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = cytosine phosphorothioate
  modified_base 6: mod_base = OTHER; note = beta-D-oxy LNA cytosine phosphorothioate
  modified_base 7: mod_base = OTHER; note = thymine phosphorothioate
  modified_base 8: mod_base = OTHER; note = beta-D-oxy LNA guanine phosphorothioate
  modified_base 9: mod_base = OTHER; note = cytosine phosphorothioate
  modified_base 10: mod_base = OTHER; note = adenine phosphorothioate
  modified_base 11: mod_base = OTHER; note = beta-D-oxy LNA guanine phosphorothioate
  modified_base 12: mod_base = OTHER; note = guanine phosphorothioate
  modified_base 13: mod_base = OTHER; note = adenine phosphorothioate
  modified_base 14: mod_base = OTHER; note = beta-D-oxy LNA adenine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA cytosine phosphorothioate
  modified_base 18: mod_base = OTHER; note = beta-D-oxy LNA thymine
cccacctgcaggaactct

SEQ ID NO: 252 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA guanine phosphorothioate
  modified_base 4: mod_base = OTHER; note = cytosine phosphorothioate
  modified_base 5: mod_base = OTHER; note = cytosine phosphorothioate
  modified_base 6: mod_base = OTHER; note = beta-D-oxy LNA cytosine phosphorothioate
  modified_base 7: mod_base = OTHER; note = adenine phosphorothioate
  modified_base 8: mod_base = OTHER; note = cytosine phosphorothioate
  modified_base 9: mod_base = OTHER; note = beta-D-oxy LNA cytosine phosphorothioate
  modified_base 10: mod_base = OTHER; note = thymine phosphorothioate
  modified_base 11: mod_base = OTHER; note = beta-D-oxy LNA guanine phosphorothioate
  modified_base 12: mod_base = OTHER; note = cytosine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = adenine phosphorothioate
  modified_base 17: mod_base = OTHER; note = beta-D-oxy LNA adenine phosphorothioate
  modified_base 18: mod_base = OTHER; note = beta-D-oxy LNA cytosine
ccgcccacctgcaggaac

SEQ ID NO: 253 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = thymine phosphorothioate
  modified_base 3: mod_base = OTHER; note = beta-D-oxy LNA adenine phosphorothioate
  modified_base 4: mod_base = OTHER; note = cytosine phosphorothioate
  modified_base 5: mod_base = OTHER; note = cytosine phosphorothioate
  modified_base 6: mod_base = OTHER; note = beta-D-oxy LNA guanine phosphorothioate
  modified_base 7: mod_base = OTHER; note = cytosine phosphorothioate
  modified_base 8: mod_base = OTHER; note = cytosine phosphorothioate
  modified_base 9: mod_base = OTHER; note = beta-D-oxy LNA cytosine phosphorothioate
  modified_base 10: mod_base = OTHER; note = adenine phosphorothioate
  modified_base 11: mod_base = OTHER; note = cytosine phosphorothioate
  modified_base 12: mod_base = OTHER; note = beta-D-oxy LNA cytosine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA guanine
ttaccgcccacctgcagg

SEQ ID NO: 254 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = adenine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = thymine phosphorothioate
  modified_base 6: mod_base = OTHER; note = beta-D-oxy LNA adenine phosphorothioate
  modified_base 7: mod_base = OTHER; note = cytosine phosphorothioate
  modified_base 8: mod_base = OTHER; note = cytosine phosphorothioate
  modified_base 9: mod_base = OTHER; note = beta-D-oxy LNA guanine phosphorothioate
  modified_base 10: mod_base = OTHER; note = cytosine phosphorothioate
  modified_base 11: mod_base = OTHER; note = cytosine phosphorothioate
  modified_base 12: mod_base = OTHER; note = beta-D-oxy LNA cytosine phosphorothioate
  modified_base 13: mod_base = OTHER; note = adenine phosphorothioate
  modified_base 14: mod_base = OTHER; note = cytosine phosphorothioate
  modified_base 15: mod_base = OTHER; note = beta-D-oxy LNA cytosine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA cytosine
gacttaccgcccacctgc

SEQ ID NO: 255 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = guanine phosphorothioate
  modified_base 3: mod_base = OTHER; note = thymine phosphorothioate
  modified_base 4: mod_base = OTHER; note = beta-D-oxy LNA guanine phosphorothioate
  modified_base 5: mod_base = OTHER; note = adenine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = thymine phosphorothioate
  modified_base 9: mod_base = OTHER; note = beta-D-oxy LNA adenine phosphorothioate
  modified_base 10: mod_base = OTHER; note = cytosine phosphorothioate
  modified_base 11: mod_base = OTHER; note = cytosine phosphorothioate
  modified_base 12: mod_base = OTHER; note = beta-D-oxy LNA guanine phosphorothioate
  modified_base 13: mod_base = OTHER; note = cytosine phosphorothioate
  modified_base 14: mod_base = OTHER; note = cytosine phosphorothioate
  modified_base 15: mod_base = OTHER; note = beta-D-oxy LNA cytosine phosphorothioate
  modified_base 16: mod_base = OTHER; note = adenine phosphorothioate
  modified_base 17: mod_base = OTHER; note = beta-D-oxy LNA cytosine phosphorothioate
  modified_base 18: mod_base = OTHER; note = beta-D-oxy LNA cytosine
ggtgacttaccgcccacc

SEQ ID NO: 256 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = thymine phosphorothioate
  modified_base 3: mod_base = OTHER; note = beta-D-oxy LNA guanine phosphorothioate
  modified_base 4: mod_base = OTHER; note = guanine phosphorothioate
  modified_base 5: mod_base = OTHER; note = beta-D-oxy LNA guanine phosphorothioate
  modified_base 6: mod_base = OTHER; note = thymine phosphorothioate
  modified_base 7: mod_base = OTHER; note = guanine phosphorothioate
  modified_base 8: mod_base = OTHER; note = beta-D-oxy LNA adenine phosphorothioate
  modified_base 9: mod_base = OTHER; note = cytosine phosphorothioate
  modified_base 10: mod_base = OTHER; note = beta-D-oxy LNA thymine phosphorothioate
  modified_base 11: mod_base = OTHER; note = thymine phosphorothioate
  modified_base 12: mod_base = OTHER; note = beta-D-oxy LNA adenine phosphorothioate
  modified_base 13: mod_base = OTHER; note = cytosine phosphorothioate
  modified_base 14: mod_base = OTHER; note = cytosine phosphorothioate
  modified_base 15: mod_base = OTHER; note = beta-D-oxy LNA guanine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA cytosine
atgggtgacttaccgccc

SEQ ID NO: 257 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = guanine phosphorothioate
  modified_base 3: mod_base = OTHER; note = guanine phosphorothioate
  modified_base 4: mod_base = OTHER; note = beta-D-oxy LNA adenine phosphorothioate
  modified_base 5: mod_base = OTHER; note = beta-D-oxy LNA thymine phosphorothioate
  modified_base 6: mod_base = OTHER; note = guanine phosphorothioate
  modified_base 7: mod_base = OTHER; note = guanine phosphorothioate
  modified_base 8: mod_base = OTHER; note = beta-D-oxy LNA guanine phosphorothioate
  modified_base 9: mod_base = OTHER; note = beta-D-oxy LNA thymine phosphorothioate
  modified_base 10: mod_base = OTHER; note = guanine phosphorothioate
  modified_base 11: mod_base = OTHER; note = adenine phosphorothioate
  modified_base 12: mod_base = OTHER; note = beta-D-oxy LNA cytosine phosphorothioate
  modified_base 13: mod_base = OTHER; note = beta-D-oxy LNA thymine phosphorothioate
  modified_base 14: mod_base = OTHER; note = thymine phosphorothioate
  modified_base 15: mod_base = OTHER; note = beta-D-oxy LNA adenine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA guanine
aggatgggtgacttaccg

SEQ ID NO: 258 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = adenine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = beta-D-oxy LNA guanine phosphorothioate
  modified_base 6: mod_base = OTHER; note = guanine phosphorothioate
  modified_base 7: mod_base = OTHER; note = beta-D-oxy LNA adenine phosphorothioate
  modified_base 8: mod_base = OTHER; note = beta-D-oxy LNA thymine phosphorothioate
  modified_base 9: mod_base = OTHER; note = guanine phosphorothioate
  modified_base 10: mod_base = OTHER; note = guanine phosphorothioate
  modified_base 11: mod_base = OTHER; note = beta-D-oxy LNA guanine phosphorothioate
  modified_base 12: mod_base = OTHER; note = beta-D-oxy LNA thymine phosphorothioate
  modified_base 13: mod_base = OTHER; note = guanine phosphorothioate
  modified_base 14: mod_base = OTHER; note = beta-D-oxy LNA adenine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA thymine phosphorothioate
  modified_base 18: mod_base = OTHER; note = beta-D-oxy LNA adenine
tacaggatgggtgactta

SEQ ID NO: 259 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA thymine phosphorothioate
  modified_base 5: mod_base = OTHER; note = adenine phosphorothioate
  modified_base 6: mod_base = OTHER; note = cytosine phosphorothioate
  modified_base 7: mod_base = OTHER; note = beta-D-oxy LNA adenine phosphorothioate
  modified_base 8: mod_base = OTHER; note = guanine phosphorothioate
  modified_base 9: mod_base = OTHER; note = guanine phosphorothioate
  modified_base 10: mod_base = OTHER; note = beta-D-oxy LNA adenine phosphorothioate
  modified_base 11: mod_base = OTHER; note = beta-D-oxy LNA thymine phosphorothioate
  modified_base 12: mod_base = OTHER; note = guanine phosphorothioate
  modified_base 13: mod_base = OTHER; note = beta-D-oxy LNA guanine phosphorothioate
  modified_base 14: mod_base = OTHER; note = guanine phosphorothioate
  modified_base 15: mod_base = OTHER; note = beta-D-oxy LNA thymine phosphorothioate
  modified_base 16: mod_base = OTHER; note = guanine phosphorothioate
  modified_base 17: mod_base = OTHER; note = beta-D-oxy LNA adenine phosphorothioate
  modified_base 18: mod_base = OTHER; note = beta-D-oxy LNA cytosine
ccctacaggatgggtgac

SEQ ID NO: 260 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = adenine phosphorothioate
  modified_base 3: mod_base = OTHER; note = guanine phosphorothioate
  modified_base 4: mod_base = OTHER; note = beta-D-oxy LNA cytosine phosphorothioate
  modified_base 5: mod_base = OTHER; note = cytosine phosphorothioate
  modified_base 6: mod_base = OTHER; note = cytosine phosphorothioate
  modified_base 7: mod_base = OTHER; note = beta-D-oxy LNA thymine phosphorothioate
  modified_base 8: mod_base = OTHER; note = adenine phosphorothioate
  modified_base 9: mod_base = OTHER; note = cytosine phosphorothioate
  modified_base 10: mod_base = OTHER; note = beta-D-oxy LNA adenine phosphorothioate
  modified_base 11: mod_base = OTHER; note = guanine phosphorothioate
  modified_base 12: mod_base = OTHER; note = guanine phosphorothioate
  modified_base 13: mod_base = OTHER; note = beta-D-oxy LNA adenine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA guanine phosphorothioate
  modified_base 18: mod_base = OTHER; note = beta-D-oxy LNA thymine
cagccctacaggatgggt

SEQ ID NO: 261 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = guanine phosphorothioate
  modified_base 3: mod_base = OTHER; note = cytosine phosphorothioate
  modified_base 4: mod_base = OTHER; note = beta-D-oxy LNA cytosine phosphorothioate
  modified_base 5: mod_base = OTHER; note = adenine phosphorothioate
  modified_base 6: mod_base = OTHER; note = beta-D-oxy LNA guanine phosphorothioate
  modified_base 7: mod_base = OTHER; note = cytosine phosphorothioate
  modified_base 8: mod_base = OTHER; note = beta-D-oxy LNA cytosine phosphorothioate
  modified_base 9: mod_base = OTHER; note = cytosine phosphorothioate
  modified_base 10: mod_base = OTHER; note = thymine phosphorothioate
  modified_base 11: mod_base = OTHER; note = beta-D-oxy LNA adenine phosphorothioate
  modified_base 12: mod_base = OTHER; note = cytosine phosphorothioate
  modified_base 13: mod_base = OTHER; note = adenine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = adenine phosphorothioate
  modified_base 17: mod_base = OTHER; note = beta-D-oxy LNA thymine phosphorothioate
  modified_base 18: mod_base = OTHER; note = beta-D-oxy LNA guanine
ggccagccctacaggatg

SEQ ID NO: 262 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA thymine phosphorothioate
  modified_base 3: mod_base = OTHER; note = guanine phosphorothioate
  modified_base 4: mod_base = OTHER; note = guanine phosphorothioate
  modified_base 5: mod_base = OTHER; note = beta-D-oxy LNA guanine phosphorothioate
  modified_base 6: mod_base = OTHER; note = cytosine phosphorothioate
  modified_base 7: mod_base = OTHER; note = cytosine phosphorothioate
  modified_base 8: mod_base = OTHER; note = beta-D-oxy LNA adenine phosphorothioate
  modified_base 9: mod_base = OTHER; note = guanine phosphorothioate
  modified_base 10: mod_base = OTHER; note = cytosine phosphorothioate
  modified_base 11: mod_base = OTHER; note = beta-D-oxy LNA cytosine phosphorothioate
  modified_base 12: mod_base = OTHER; note = cytosine phosphorothioate
  modified_base 13: mod_base = OTHER; note = thymine phosphorothioate
  modified_base 14: mod_base = OTHER; note = beta-D-oxy LNA adenine phosphorothioate
  modified_base 15: mod_base = OTHER; note = cytosine phosphorothioate
  modified_base 16: mod_base = OTHER; note = adenine phosphorothioate
  modified_base 17: mod_base = OTHER; note = beta-D-oxy LNA guanine phosphorothioate
  modified_base 18: mod_base = OTHER; note = beta-D-oxy LNA guanine
atgggccagccctacagg

SEQ ID NO: 263 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = guanine phosphorothioate
  modified_base 3: mod_base = OTHER; note = guanine phosphorothioate
  modified_base 4: mod_base = OTHER; note = beta-D-oxy LNA adenine phosphorothioate
  modified_base 5: mod_base = OTHER; note = beta-D-oxy LNA thymine phosphorothioate
  modified_base 6: mod_base = OTHER; note = guanine phosphorothioate
  modified_base 7: mod_base = OTHER; note = guanine phosphorothioate
  modified_base 8: mod_base = OTHER; note = beta-D-oxy LNA guanine phosphorothioate
  modified_base 9: mod_base = OTHER; note = cytosine phosphorothioate
  modified_base 10: mod_base = OTHER; note = cytosine phosphorothioate
  modified_base 11: mod_base = OTHER; note = beta-D-oxy LNA adenine phosphorothioate
  modified_base 12: mod_base = OTHER; note = guanine phosphorothioate
  modified_base 13: mod_base = OTHER; note = cytosine phosphorothioate
  modified_base 14: mod_base = OTHER; note = beta-D-oxy LNA cytosine phosphorothioate
  modified_base 15: mod_base = OTHER; note = cytosine phosphorothioate
  modified_base 16: mod_base = OTHER; note = thymine phosphorothioate
  modified_base 17: mod_base = OTHER; note = beta-D-oxy LNA adenine phosphorothioate
  modified_base 18: mod_base = OTHER; note = beta-D-oxy LNA cytosine
tggatgggccagccctac

SEQ ID NO: 264 - DNA, 18 bp, synthetic construct
  source 1..18: mol_type = other DNA
  modified_base 1: mod_base = OTHER; note = beta-D-oxy LNA cytosine phosphorothioate
  modified_base 2: mod_base = OTHER; note = beta-D-oxy LNA thymine phosphorothioate
  modified_base 3: mod_base = OTHER; note = thymine phosphorothioate
  modified_base 4: mod_base = OTHER; note = thymine phosphorothioate
  modified_base 5: mod_base = OTHER; note = beta-D-oxy LNA guanine phosphorothioate
  modified_base 6: mod_base = OTHER; note = guanine phosphorothioate
  modified_base 7: mod_base = OTHER; note = adenine phosphorothioate
  modified_base 8: mod_base = OTHER; note = beta-D-oxy LNA thymine phosphorothioate
  modified_base 9: mod_base = OTHER; note = guanine phosphorothioate
  modified_base 10: mod_base = OTHER; note = guanine phosphorothioate
  modified_base 11: mod_base = OTHER; note = beta-D-oxy LNA guanine phosphorothioate
  modified_base 12: mod_base = OTHER; note = cytosine phosphorothioate
  modified_base 13: mod_base = OTHER; note = cytosine phosphorothioate
  modified_base 14: mod_base = OTHER; note = beta-D-oxy LNA adenine phosphorothioate
  modified_base 15: mod_base = OTHER; note = guanine phosphorothioate
  modified_base 16: mod_base = OTHER; note = cytosine phosphorothioate
  modified_base 17: mod_base = OTHER; note = beta-D-oxy LNA cytosine phosphorothioate
  modified_base 18: mod_base = OTHER; note = beta-D-oxy LNA cytosine
ctttggatgggccagccc

SEQ ID NO: 265 - DNA, 18 bp, synthetic construct
  source 1..18: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = adenine phosphorothioate
  modified_base 4: mod_base = OTHER; note = beta-D-oxy LNA cytosine phosphorothioate
  modified_base 5: mod_base = OTHER; note = beta-D-oxy LNA thymine phosphorothioate
  modified_base 6: mod_base = OTHER; note = thymine phosphorothioate
  modified_base 7: mod_base = OTHER; note = thymine phosphorothioate
  modified_base 8: mod_base = OTHER; note = beta-D-oxy LNA guanine phosphorothioate
  modified_base 9: mod_base = OTHER; note = guanine phosphorothioate
  modified_base 10: mod_base = OTHER; note = beta-D-oxy LNA adenine phosphorothioate
  modified_base 11: mod_base = OTHER; note = beta-D-oxy LNA thymine phosphorothioate
  modified_base 12: mod_base = OTHER; note = guanine phosphorothioate
  modified_base 13: mod_base = OTHER; note = guanine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = cytosine phosphorothioate
  modified_base 17: mod_base = OTHER; note = beta-D-oxy LNA adenine phosphorothioate
  modified_base 18: mod_base = OTHER; note = beta-D-oxy LNA guanine
tcactttggatgggccag

SEQ ID NO: 266 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA thymine phosphorothioate
  modified_base 3: mod_base = OTHER; note = guanine phosphorothioate
  modified_base 4: mod_base = OTHER; note = thymine phosphorothioate
  modified_base 5: mod_base = OTHER; note = beta-D-oxy LNA cytosine phosphorothioate
  modified_base 6: mod_base = OTHER; note = adenine phosphorothioate
  modified_base 7: mod_base = OTHER; note = cytosine phosphorothioate
  modified_base 8: mod_base = OTHER; note = beta-D-oxy LNA thymine phosphorothioate
  modified_base 9: mod_base = OTHER; note = beta-D-oxy LNA thymine phosphorothioate
  modified_base 10: mod_base = OTHER; note = thymine phosphorothioate
  modified_base 11: mod_base = OTHER; note = beta-D-oxy LNA guanine phosphorothioate
  modified_base 12: mod_base = OTHER; note = guanine phosphorothioate
  modified_base 13: mod_base = OTHER; note = beta-D-oxy LNA adenine phosphorothioate
  modified_base 14: mod_base = OTHER; note = beta-D-oxy LNA thymine phosphorothioate
  modified_base 15: mod_base = OTHER; note = guanine phosphorothioate
  modified_base 16: mod_base = OTHER; note = guanine phosphorothioate
  modified_base 17: mod_base = OTHER; note = beta-D-oxy LNA guanine phosphorothioate
  modified_base 18: mod_base = OTHER; note = beta-D-oxy LNA cytosine
atgtcactttggatgggc

SEQ ID NO: 267 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = beta-D-oxy LNA thymine phosphorothioate
  modified_base 6: mod_base = OTHER; note = guanine phosphorothioate
  modified_base 7: mod_base = OTHER; note = thymine phosphorothioate
  modified_base 8: mod_base = OTHER; note = beta-D-oxy LNA cytosine phosphorothioate
  modified_base 9: mod_base = OTHER; note = beta-D-oxy LNA adenine phosphorothioate
  modified_base 10: mod_base = OTHER; note = cytosine phosphorothioate
  modified_base 11: mod_base = OTHER; note = beta-D-oxy LNA thymine phosphorothioate
  modified_base 12: mod_base = OTHER; note = beta-D-oxy LNA thymine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = guanine phosphorothioate
  modified_base 16: mod_base = OTHER; note = adenine phosphorothioate
  modified_base 17: mod_base = OTHER; note = beta-D-oxy LNA thymine phosphorothioate
  modified_base 18: mod_base = OTHER; note = beta-D-oxy LNA guanine
gccatgtcactttggatg

SEQ ID NO: 268 - DNA, 18 bp, synthetic construct
  source 1..18: mol_type = other DNA
  modified_base 1: mod_base = OTHER; note = beta-D-oxy LNA adenine phosphorothioate
  modified_base 2: mod_base = OTHER; note = beta-D-oxy LNA adenine phosphorothioate
  modified_base 3: mod_base = OTHER; note = thymine phosphorothioate
  modified_base 4: mod_base = OTHER; note = guanine phosphorothioate
  modified_base 5: mod_base = OTHER; note = beta-D-oxy LNA cytosine phosphorothioate
  modified_base 6: mod_base = OTHER; note = beta-D-oxy LNA cytosine phosphorothioate
  modified_base 7: mod_base = OTHER; note = adenine phosphorothioate
  modified_base 8: mod_base = OTHER; note = thymine phosphorothioate
  modified_base 9: mod_base = OTHER; note = beta-D-oxy LNA guanine phosphorothioate
  modified_base 10: mod_base = OTHER; note = beta-D-oxy LNA thymine phosphorothioate
  modified_base 11: mod_base = OTHER; note = cytosine phosphorothioate
  modified_base 12: mod_base = OTHER; note = adenine phosphorothioate
  modified_base 13: mod_base = OTHER; note = beta-D-oxy LNA cytosine phosphorothioate
  modified_base 14: mod_base = OTHER; note = beta-D-oxy LNA thymine phosphorothioate
  modified_base 15: mod_base = OTHER; note = thymine phosphorothioate
  modified_base 16: mod_base = OTHER; note = thymine phosphorothioate
  modified_base 17: mod_base = OTHER; note = beta-D-oxy LNA guanine phosphorothioate
  modified_base 18: mod_base = OTHER; note = beta-D-oxy LNA guanine
aatgccatgtcactttgg

SEQ ID NO: 269 - DNA, 20 bp, synthetic construct
  source 1..20: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = adenine phosphorothioate
  modified_base 4: mod_base = OTHER; note = beta-D-oxy LNA guanine phosphorothioate
  modified_base 5: mod_base = OTHER; note = guanine phosphorothioate
  modified_base 6: mod_base = OTHER; note = thymine phosphorothioate
  modified_base 7: mod_base = OTHER; note = beta-D-oxy LNA adenine phosphorothioate
  modified_base 8: mod_base = OTHER; note = adenine phosphorothioate
  modified_base 9: mod_base = OTHER; note = thymine phosphorothioate
  modified_base 10: mod_base = OTHER; note = beta-D-oxy LNA thymine phosphorothioate
  modified_base 11: mod_base = OTHER; note = guanine phosphorothioate
  modified_base 12: mod_base = OTHER; note = thymine phosphorothioate
  modified_base 13: mod_base = OTHER; note = beta-D-oxy LNA guanine phosphorothioate
  modified_base 14: mod_base = OTHER; note = cytosine phosphorothioate
  modified_base 15: mod_base = OTHER; note = cytosine phosphorothioate
  modified_base 16: mod_base = OTHER; note = beta-D-oxy LNA thymine phosphorothioate
  modified_base 17: mod_base = OTHER; note = guanine phosphorothioate
  modified_base 18: mod_base = OTHER; note = adenine phosphorothioate
  modified_base 19: mod_base = OTHER; note = beta-D-oxy LNA guanine phosphorothioate
  modified_base 20: mod_base = OTHER; note = beta-D-oxy LNA guanine
ccaggtaattgtgcctgagg

SEQ ID NO: 270 - DNA, 20 bp, synthetic construct
  source 1..20: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA adenine phosphorothioate
  modified_base 5: mod_base = OTHER; note = guanine phosphorothioate
  modified_base 6: mod_base = OTHER; note = beta-D-oxy LNA guanine phosphorothioate
  modified_base 7: mod_base = OTHER; note = thymine phosphorothioate
  modified_base 8: mod_base = OTHER; note = adenine phosphorothioate
  modified_base 9: mod_base = OTHER; note = beta-D-oxy LNA adenine phosphorothioate
  modified_base 10: mod_base = OTHER; note = thymine phosphorothioate
  modified_base 11: mod_base = OTHER; note = thymine phosphorothioate
  modified_base 12: mod_base = OTHER; note = beta-D-oxy LNA guanine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = cytosine phosphorothioate
  modified_base 16: mod_base = OTHER; note = cytosine phosphorothioate
  modified_base 17: mod_base = OTHER; note = beta-D-oxy LNA thymine phosphorothioate
  modified_base 18: mod_base = OTHER; note = guanine phosphorothioate
  modified_base 19: mod_base = OTHER; note = beta-D-oxy LNA adenine phosphorothioate
  modified_base 20: mod_base = OTHER; note = beta-D-oxy LNA guanine
cccaggtaattgtgcctgag

SEQ ID NO: 271 - DNA, 20 bp, synthetic construct
  source 1..20: mol_type = other DNA
  modified_base 1: mod_base = OTHER; note = beta-D-oxy LNA thymine phosphorothioate
  modified_base 2: mod_base = OTHER; note = cytosine phosphorothioate
  modified_base 3: mod_base = OTHER; note = cytosine phosphorothioate
  modified_base 4: mod_base = OTHER; note = beta-D-oxy LNA cytosine phosphorothioate
  modified_base 5: mod_base = OTHER; note = adenine phosphorothioate
  modified_base 6: mod_base = OTHER; note = guanine phosphorothioate
  modified_base 7: mod_base = OTHER; note = beta-D-oxy LNA guanine phosphorothioate
  modified_base 8: mod_base = OTHER; note = thymine phosphorothioate
  modified_base 9: mod_base = OTHER; note = adenine phosphorothioate
  modified_base 10: mod_base = OTHER; note = beta-D-oxy LNA adenine phosphorothioate
  modified_base 11: mod_base = OTHER; note = thymine phosphorothioate
  modified_base 12: mod_base = OTHER; note = thymine phosphorothioate
  modified_base 13: mod_base = OTHER; note = beta-D-oxy LNA guanine phosphorothioate
  modified_base 14: mod_base = OTHER; note = thymine phosphorothioate
  modified_base 15: mod_base = OTHER; note = guanine phosphorothioate
  modified_base 16: mod_base = OTHER; note = beta-D-oxy LNA cytosine phosphorothioate
  modified_base 17: mod_base = OTHER; note = cytosine phosphorothioate
  modified_base 18: mod_base = OTHER; note = thymine phosphorothioate
  modified_base 19: mod_base = OTHER; note = beta-D-oxy LNA guanine phosphorothioate
  modified_base 20: mod_base = OTHER; note = beta-D-oxy LNA adenine
tcccaggtaattgtgcctga

SEQ ID NO: 272 - DNA, 20 bp, synthetic construct
  source 1..20: mol_type = other DNA
  modified_base 1: mod_base = OTHER; note = beta-D-oxy LNA cytosine phosphorothioate
  modified_base 2: mod_base = OTHER; note = thymine phosphorothioate
  modified_base 3: mod_base = OTHER; note = cytosine phosphorothioate
  modified_base 4: mod_base = OTHER; note = beta-D-oxy LNA cytosine phosphorothioate
  modified_base 5: mod_base = OTHER; note = cytosine phosphorothioate
  modified_base 6: mod_base = OTHER; note = adenine phosphorothioate
  modified_base 7: mod_base = OTHER; note = beta-D-oxy LNA guanine phosphorothioate
  modified_base 8: mod_base = OTHER; note = guanine phosphorothioate
  modified_base 9: mod_base = OTHER; note = thymine phosphorothioate
  modified_base 10: mod_base = OTHER; note = beta-D-oxy LNA adenine phosphorothioate
  modified_base 11: mod_base = OTHER; note = adenine phosphorothioate
  modified_base 12: mod_base = OTHER; note = thymine phosphorothioate
  modified_base 13: mod_base = OTHER; note = beta-D-oxy LNA thymine phosphorothioate
  modified_base 14: mod_base = OTHER; note = guanine phosphorothioate
  modified_base 15: mod_base = OTHER; note = thymine phosphorothioate
  modified_base 16: mod_base = OTHER; note = beta-D-oxy LNA guanine phosphorothioate
  modified_base 17: mod_base = OTHER; note = cytosine phosphorothioate
  modified_base 18: mod_base = OTHER; note = cytosine phosphorothioate
  modified_base 19: mod_base = OTHER; note = beta-D-oxy LNA thymine phosphorothioate
  modified_base 20: mod_base = OTHER; note = beta-D-oxy LNA guanine
ctcccaggtaattgtgcctg

SEQ ID NO: 273 - DNA, 16 bp, synthetic construct
  source 1..16: mol_type = other DNA
  modified_base 1: mod_base = OTHER; note = beta-D-oxy LNA guanine phosphorothioate
  modified_base 2: mod_base = OTHER; note = beta-D-oxy LNA adenine phosphorothioate
  modified_base 3: mod_base = OTHER; note = beta-D-oxy LNA thymine phosphorothioate
  modified_base 4: mod_base = OTHER; note = thymine phosphorothioate
  modified_base 5: mod_base = OTHER; note = thymine phosphorothioate
  modified_base 6: mod_base = OTHER; note = cytosine phosphorothioate
  modified_base 7: mod_base = OTHER; note = cytosine phosphorothioate
  modified_base 8: mod_base = OTHER; note = cytosine phosphorothioate
  modified_base 9: mod_base = OTHER; note = adenine phosphorothioate
  modified_base 10: mod_base = OTHER; note = guanine phosphorothioate
  modified_base 11: mod_base = OTHER; note = thymine phosphorothioate
  modified_base 12: mod_base = OTHER; note = guanine phosphorothioate
  modified_base 13: mod_base = OTHER; note = cytosine phosphorothioate
  modified_base 14: mod_base = OTHER; note = beta-D-oxy LNA cytosine phosphorothioate
  modified_base 15: mod_base = OTHER; note = beta-D-oxy LNA adenine phosphorothioate
  modified_base 16: mod_base = OTHER; note = beta-D-oxy LNA thymine
gatttcccagtgccat

SEQ ID NO: 274 - DNA, 16 bp, synthetic construct
  source 1..16: mol_type = other DNA
  modified_base 1: mod_base = OTHER; note = beta-D-oxy LNA guanine phosphorothioate
  modified_base 2: mod_base = OTHER; note = beta-D-oxy LNA cytosine phosphorothioate
  modified_base 3: mod_base = OTHER; note = beta-D-oxy LNA adenine phosphorothioate
  modified_base 4: mod_base = OTHER; note = cytosine phosphorothioate
  modified_base 5: mod_base = OTHER; note = thymine phosphorothioate
  modified_base 6: mod_base = OTHER; note = cytosine phosphorothioate
  modified_base 7: mod_base = OTHER; note = guanine phosphorothioate
  modified_base 8: mod_base = OTHER; note = adenine phosphorothioate
  modified_base 9: mod_base = OTHER; note = cytosine phosphorothioate
  modified_base 10: mod_base = OTHER; note = adenine phosphorothioate
  modified_base 11: mod_base = OTHER; note = cytosine phosphorothioate
  modified_base 12: mod_base = OTHER; note = thymine phosphorothioate
  modified_base 13: mod_base = OTHER; note = thymine phosphorothioate
  modified_base 14: mod_base = OTHER; note = beta-D-oxy LNA guanine phosphorothioate
  modified_base 15: mod_base = OTHER; note = beta-D-oxy LNA guanine phosphorothioate
  modified_base 16: mod_base = OTHER; note = beta-D-oxy LNA thymine
gcactcgacacttggt

SEQ ID NO: 275 - DNA, 18 bp, synthetic construct
  source 1..18: mol_type = other DNA
  modified_base 1: mod_base = OTHER; note = beta-D-oxy LNA guanine phosphorothioate
  modified_base 2: mod_base = OTHER; note = beta-D-oxy LNA thymine phosphorothioate
  modified_base 3: mod_base = OTHER; note = beta-D-oxy LNA adenine phosphorothioate
  modified_base 4: mod_base = OTHER; note = cytosine phosphorothioate
  modified_base 5: mod_base = OTHER; note = beta-D-oxy LNA adenine phosphorothioate
  modified_base 6: mod_base = OTHER; note = beta-D-oxy LNA thymine phosphorothioate
  modified_base 7: mod_base = OTHER; note = cytosine phosphorothioate
  modified_base 8: mod_base = OTHER; note = beta-D-oxy LNA cytosine phosphorothioate
  modified_base 9: mod_base = OTHER; note = beta-D-oxy LNA adenine phosphorothioate
  modified_base 10: mod_base = OTHER; note = beta-D-oxy LNA cytosine phosphorothioate
  modified_base 11: mod_base = OTHER; note = thymine phosphorothioate
  modified_base 12: mod_base = OTHER; note = thymine phosphorothioate
  modified_base 13: mod_base = OTHER; note = beta-D-oxy LNA adenine phosphorothioate
  modified_base 14: mod_base = OTHER; note = beta-D-oxy LNA thymine phosphorothioate
  modified_base 15: mod_base = OTHER; note = thymine phosphorothioate
  modified_base 16: mod_base = OTHER; note = beta-D-oxy LNA cytosine phosphorothioate
  modified_base 17: mod_base = OTHER; note = beta-D-oxy LNA adenine phosphorothioate
  modified_base 18: mod_base = OTHER; note = beta-D-oxy LNA adenine
gtacatccacttattcaa

SEQ ID NO: 276 - DNA, 20 bp, synthetic construct
  source 1..20: mol_type = other DNA
  modified_base 1: mod_base = OTHER; note = beta-D-oxy LNA cytosine phosphorothioate
  modified_base 2: mod_base = OTHER; note = cytosine phosphorothioate
  modified_base 3: mod_base = OTHER; note = beta-D-oxy LNA adenine phosphorothioate
  modified_base 4: mod_base = OTHER; note = beta-D-oxy LNA adenine phosphorothioate
  modified_base 5: mod_base = OTHER; note = beta-D-oxy LNA adenine phosphorothioate
  modified_base 6: mod_base = OTHER; note = thymine phosphorothioate
  modified_base 7: mod_base = OTHER; note = cytosine phosphorothioate
  modified_base 8: mod_base = OTHER; note = thymine phosphorothioate
  modified_base 9: mod_base = OTHER; note = thymine phosphorothioate
  modified_base 10: mod_base = OTHER; note = adenine phosphorothioate
  modified_base 11: mod_base = OTHER; note = thymine phosphorothioate
  modified_base 12: mod_base = OTHER; note = adenine phosphorothioate
  modified_base 13: mod_base = OTHER; note = adenine phosphorothioate
  modified_base 14: mod_base = OTHER; note = thymine phosphorothioate
  modified_base 15: mod_base = OTHER; note = adenine phosphorothioate
  modified_base 16: mod_base = OTHER; note = beta-D-oxy LNA adenine phosphorothioate
  modified_base 17: mod_base = OTHER; note = beta-D-oxy LNA cytosine phosphorothioate
  modified_base 18: mod_base = OTHER; note = thymine phosphorothioate
  modified_base 19: mod_base = OTHER; note = beta-D-oxy LNA adenine phosphorothioate
  modified_base 20: mod_base = OTHER; note = beta-D-oxy LNA cytosine
ccaaatcttataataactac

(19) United States
(12) Patent Application Publication
(10) Pub. No.: US 2023/0122753 A1
(43) Pub. Date: Apr. 20, 2023

(54) OLIGONUCLEOTIDES FOR MODULATING CD73 EXON 7 SPLICING

(71) Applicant: Hoffmann-La Roche Inc., Little Falls, NJ (US)

(72) Inventors: Mads Aaboe JENSEN, Farum (DK); Lars JOENSON, Viby Sjaelland (DK); Gianluigi LICHINCHI, Basel (CH); Jonas VIKESAA, Fredensborg (DK)

(21) Appl. No.: 17/822,668

(22) Filed: Aug. 26, 2022

Related U.S. Application Data
(63) Continuation of application No. PCT/EP2021/054640, filed on Feb. 25, 2021.

(30) Foreign Application Priority Data
Feb. 28, 2020 (EP) 20159977.6

(51) Int. Cl. C12N 15/113; A61P 35/00

(52) U.S. Cl. CPC C12N 15/113; A61P 35/00; C12N 2310/11; C12N 2310/351

(57) ABSTRACT

The present invention relates to antisense oligonucleotides that are complementary to mammalian CD73 (NT5E) pre-mRNA, wherein the antisense oligonucleotides are capable of modulating the splicing of mammalian CD73 pre-mRNA exon 7. Splice modulation of mammalian CD73 exon 7 is beneficial for a range of medical disorders, including disorders in the field of immune-oncology.

### FIELD OF INVENTION

The present invention relates to antisense oligonucleotides that are complementary to mammalian CD73 (NT5E) pre-mRNA, wherein the antisense oligonucleotides are capable of modulating the splicing of mammalian CD73 pre-mRNA exon 7. Splice modulation of mammalian CD73 exon 7 is beneficial for a range of medical disorders, including disorders in the field of immune-oncology.

### BACKGROUND

CD73 is a glycosylphosphatidylinositol (GPI)-anchored extracellular membrane protein. It functions as homodimer and possesses extracellular ecto-5′-nucleotidase activity responsible for the hydrolysis of adenosine-monophosphate (AMP) to adenosine (Ado) and free phosphate (Pi). CD73 is widely expressed in several tissues and cell types, including immune cells, endothelial cells and cancer cells (Resta et al., 1993; Snider et al., 2014). Production of high levels of Ado is one of the mechanisms associated with cancer immune evasion. Fast proliferating cells are well characterized by increased frequency of apoptosis, which—among other events—leads to the release of adenosine-triphosphate (ATP) in the tumor microenvironment (TME) (Silva-Vilches et al., 2018). The elevated local ATP concentration found in the TME contributes to the generation of a pro-inflammatory environment, which in turn enhances the anti-cancer functions of different immune cells. The conversion of ATP to Ado is catalyzed by the sequential activities of CD39 and CD73. Conversely to the molecular signaling activated by extracellular ATP, Ado generated by the CD39/CD73 axis elicits inhibition of dendritic cell activation, cytokine production, and T cell proliferation. Ado exerts its immunosuppressive effects by activation of G-protein coupled adenosine receptor A2AR (Fredholm et al., 2001; de Andrade Mello et al., 2017). Moreover, several cancer types are characterized by upregulation of CD73 expression, and this feature correlates with poor prognosis (Loi et al., 2013; Leclerc et al., 2016).

A previous study (Snider et al. 2014) has reported that the CD73 isoform lacking exon 7 (CD73 Δ7) possesses dominant negative functions. CD73 dimers—where one or both the subunits consist of CD73 Δ7—show significant reduction in protein stability. Accelerated proteasome degradation induced by CD73 Δ7 has been identified as the mechanism driving the reduction of the overall CD73 protein level. Moreover, CD73 dimers containing at least one Δ7 subunit have been shown to be catalytically inactive.

Splice switching oligonucleotides have been developed as therapeutic agents for treatment of diseases associated with aberrant pre-mRNA splicing, such as Spinraza®, a 2′-O-MOE antisense oligonucleotide, which corrects the exon 7 splicing in a disease causing allele of SMN1 pre-mRNA, and is approved for treatment of spinal muscular atrophy. See Havens et al., for a review of splice-switching antisense oligonucleotides as therapeutic drugs.

The present inventors have identified the antisense oligonucleotide target sites on the human CD73 pre-mRNA which enable the modulation of CD73 splicing to result in the elimination of the CD73 exon 7, and provide effective production of CD73 Δ7 (i.e. a CD73 mRNA which does not comprise the CD73 exon 7) for use in treatment of disorders in the field of immune-oncology.

### OBJECTIVE OF THE INVENTION

The present invention identifies novel mammalian CD73 pre-mRNA target sites and antisense oligonucleotides (ASOs) targeting said target sites wherein the antisense oligonucleotides modulate the splicing of the mammalian CD73 pre-mRNA. Advantageously, the modulation of splicing of mammalian CD73 pre-mRNA results in the production of exon 7 deficient CD73 mRNA (CD73 Δ7), and expression of a CD73 polypeptide which lacks part or all of the CD73 exon 7 encoded polypeptide.

The antisense oligonucleotides as described herein are useful in the treatment of disorders in the field of immune-oncology as disclosed herein, specifically a disease selected from the group consisting of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer and renal-cell cancer

### SUMMARY OF INVENTION

The present invention provides antisense oligonucleotides which are complementary to, and which are capable of modulating the expression of a CD73 nucleic acid, and for their use in medicine.

In one aspect, provided herein is an antisense oligonucleotide targeting a mammalian CD73 pre-mRNA, wherein the antisense oligonucleotide modulates splicing of the mammalian CD73 pre-mRNA, and comprises a contiguous nucleotide sequence of at least 10 nucleotides in length with at least 90% complementarity, such as 100% complementarity to the mammalian CD73 pre-mRNA. In one embodiment, the antisense oligonucleotide or contiguous nucleotide sequence thereof is capable of enhancing the expression of exon 7 deficient mammalian CD73 mRNA. In one embodiment, the antisense oligonucleotide or contiguous nucleotide sequence thereof is capable of reducing the expression of WT mammalian CD73 mRNA. In one embodiment, the antisense oligonucleotide or contiguous nucleotide sequence thereof is capable of enhancing the expression of exon 7 deficient mammalian CD73 mRNA and reducing the expression of WT mammalian CD73 mRNA. In one embodiment, the antisense oligonucleotide or contiguous nucleotide sequence thereof is 10-40, such as 10-25, such as 15-20 nucleotides in length. In one embodiment, the antisense oligonucleotide or contiguous nucleotide sequence thereof is complementary to, such as fully complementary to SEQ ID NO: 1. In one embodiment, the antisense oligonucleotide or contiguous nucleotide sequence thereof is complementary to, such as fully complementary to SEQ ID NO: 6. In one embodiment, the antisense oligonucleotide or contiguous nucleotide sequence thereof is complementary to, such as fully complementary a sequence selected from the group consisting of SEQ ID NO: 93-174. In one embodiment, the antisense oligonucleotide is or comprises an antisense oligonucleotide mixmer or totalmer. In one embodiment, the antisense oligonucleotide or contiguous nucleotide sequence thereof is 10-20 nucleotides in length. In one embodiment, the contiguous nucleotide sequence of the antisense oligonucleotide consists of or comprises a sequence selected from any one of SEQ ID NO: 7-88. In one embodiment, the antisense oligonucleotide consists of or comprises an oligonucleotide provided herein, such as an antisense oligonucleotide selected from the group consisting of any one of ASO ID NO: ASO-1-ASO-82.

In one aspect, provided herein is a conjugate comprising the antisense oligonucleotide of the invention, and at least one conjugate moiety covalently attached to said antisense oligonucleotide.

In one aspect, provided herein is a pharmaceutically acceptable salt of the antisense oligonucleotide, or the conjugate of the invention.

In one aspect, provided herein is a pharmaceutical composition comprising the antisense oligonucleotide, or the conjugate of the invention, and a pharmaceutically acceptable diluent, solvent, carrier, salt and/or adjuvant.

In one aspect, provided herein is an in vivo or in vitro method for modulating the splicing of a mammalian CD73 pre-mRNA in a target cell which is expressing mammalian CD73, said method comprising administering the antisense oligonucleotide, or the conjugate, or the pharmaceutically acceptable salt, or the pharmaceutical composition of the invention, in an effective amount to said cell. In one embodiment, the administration of the antisense oligonucleotide results in enhanced expression of exon 7 deficient mammalian CD73 mRNA. In one embodiment, the administration of the antisense oligonucleotide results in reduced expression of WT mammalian CD73 mRNA. In one embodiment, the administration of the antisense oligonucleotide results in enhanced expression of exon 7 deficient mammalian CD73 mRNA and reduced expression of WT mammalian CD73 mRNA. In one embodiment, the cell is a mammalian cell. In one embodiment, the mammalian cell is a human cell. In one embodiment, the mammalian CD73 mRNA is a human CD73 mRNA.

In one aspect, provided herein is a method for treating or preventing cancer comprising administering a therapeutically or prophylactically effective amount of the antisense oligonucleotide, or the conjugate, or the pharmaceutically acceptable salt, or the pharmaceutical composition of the invention, to a subject suffering from or susceptible to cancer, such as a cancer selected from the group consisting of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer, and renal-cell cancer.

In one aspect, provided herein is the antisense oligonucleotide, or the conjugate, or the pharmaceutically acceptable salt, or the pharmaceutical composition of the invention, for use as a medicament.

In one aspect, provided herein is the antisense oligonucleotide, or the conjugate, or the pharmaceutically acceptable salt, or the pharmaceutical composition of the invention, for use in the treatment of cancer, such as a cancer selected from the group consisting of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer, and renal-cell cancer.

In one aspect, provided herein is the use of the antisense oligonucleotide, or the conjugate, or the pharmaceutically acceptable salt, or the pharmaceutical composition of the invention, for the preparation of a medicament for treatment or prevention of cancer, such as a cancer selected from the group consisting of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer, and renal-cell cancer.

### SEQUENCE LISTING

The sequence listing submitted with this application is hereby incorporated by reference. In the event of a discrepancy between the sequence listing and the specification or figures, the information disclosed in the specification (including the figures) shall be deemed to be correct. It will be understood that in reference to target nucleic acids or target sequences or target sites, the sequences disclosed herein refer to DNA sequences derived from genomic or cDNA sequences, and are provided as representations of the nucleic acids in the cell, in vitro or in vivo, which may for instance be RNA molecules (for instance in the RNA target sequences uracil (U) is present in place of the thymine (T) shown in the enclosed DNA sequences). Target regions such as SEQ ID NO: 6 or target sequences such as SEQ ID NO: 93-174, include RNA sequences from the reference target sequence (such as SEQ ID NO: 1 or a naturally occurring variant thereof).

### CD73 Reference Sequences

TABLE 1

| SEQ ID NO: 1 | Homo sapiens CD73 pre-mRNA reference sequence |
|---|---|
| SEQ ID NO: 2 | Homo sapiens CD73 mRNA reference sequence (WT) |
| SEQ ID NO: 3 | Homo sapiens CD73 protein reference sequence (WT) |
| SEQ ID NO: 4 | Homo sapiens CD73 Δ7 mRNA reference sequence (Δ7) |
| SEQ ID NO: 5 | Homo sapiens CD73 Δ7 protein reference sequence (Δ7) |
| SEQ ID NO: 6 | Homo sapiens CD73 - Δ7 target region |
| SEQ ID NO: | Oligonucleotide sequence motifs |
| 7-88 | |
| SEQ ID NO: | Homo sapiens CD73 - target sequences |
| 93-174 | |

### Target CD73 Pre-mRNA Sequence Tiled by Antisense Oligonucleotides Provided Herein

SEQ ID NO: 6 (Lower case letters depict intronic
regions; upper case letters depict exon 7 region):
gcattttctcaagctattttccttcttgcctcatctgtgactaccctcag
GCACAATTACCTGGGAGAACCTGGCTGCTGTATTGCCCTTTGGAGGCACA
TTTGACCTAGTCCAGTTAAAAGGTTCCACCCTGAAGAAGGCCTTTGAGCA
TAGCGTGCACCGCTACGGCCAGTCCACTGGAGAGTTCCTGCAGGTGGGCG
gtaagtcacccatcctgtagggctggcccatccaaagtgacatggcattt

Exemplary Exon and Intron Regions of Human CD73 Pre-mRNA—as Illustrated with Reference to SEQ ID NO: 1

TABLE 2

| Exon | start_SEQ ID NO: 1 | end_SEQ ID NO: 1 |
|---|---|---|
| Ex_1 | 508 | 895 |
| Ex_2 | 17477 | 17699 |
| Ex_3 | 21654 | 21842 |
| Ex_4 | 35652 | 35849 |
| Ex_5 | 37752 | 37906 |
| Ex_6 | 39911 | 40016 |
| Ex_7 | 40925 | 41074 |
| Ex_8 | 42394 | 42594 |
| Ex_9 | 44258 | 46195 |

TABLE 3

| Intron | start_SEQ ID NO: 1 | end_SEQ ID NO: 1 |
|---|---|---|
| Int_1 | 896 | 17476 |
| Int_2 | 17700 | 21653 |
| Int_3 | 21843 | 35651 |
| Int_4 | 35850 | 37751 |
| Int_5 | 37907 | 39910 |
| Int_6 | 40017 | 40924 |
| Int_7 | 41075 | 42393 |
| Int_8 | 42595 | 44257 |

A CD73 Δ7 mRNA therefore lacks the Exon 7 region, such as exemplified by nucleotides 40925-41074 of SEQ ID NO: 1, or at least a portion thereof. In some embodiments, the entire exon 7 is lacking in the CD73 Δ7 mRNA, and therefore in some embodiments the CD73 Δ7 mRNA may be characterized by having a contiguous sequence formed across exon 6 and exon 8. In some embodiments, the CD73 Δ7 mRNA comprises exons 1 to 6, 8, and 9, but lacks exon 7.

### BRIEF DESCRIPTION OF FIGURES

FIGS. 1A and 1B. Screening of 82 mixmer ASOs for CD73 exon 7 skipping activity. FIG. 1A: A549 and Colo205 cells were treated with antisense ASOs at 5 μM for 4 days. FIG. 1 B: A549 and Colo205 cells were treated with antisense ASOs at 25 μM for 4 days. The level of exon 7 skipping (exS) is indicated as percentage of total CD73 mRNA. Pearson correlation coefficients of the activity of the ASOs in A549 and Colo205 cells are indicated (r). Empty circles represent ASOs targeting CD73 RNA, solid circle represents scramble control ASO.

FIG. 2. ASO-52 and ASO-15 induce CD73 Δ7 in a dose-dependent fashion. Colo205 cells were treated with the indicated ASOs at different concentrations for 4 days (n=2). The level of CD73 Δ7 is indicated as percentage of total CD73 mRNA.

FIG. 3. Induction of CD73 Δ7 by ASO-52 and ASO-15 reduces extracellular 5′ ecto-nucleotidase activity in a dose-dependent fashion. Colo205 cells were treated with the indicated ASOs at different concentrations for 4 days. Extracellular activity of CD73 was measured as free phosphate generated from AMP hydrolysis (n=2). Background level corresponds to free phosphate level measured in PBS-treated cells in absence of AMP.

FIGS. 4A and 4B. ASO-52 and ASO-15 reduce CD73 protein level. FIG. 4A: Colo205 cells were treated with antisense ASOs at 25 μM for 4 days. CD73 protein level was quantitated by immunoblotting on whole-cell lysate. GAPDH level was used as internal normalizer. FIG. 4B: Densitometry analysis of the Colo205 cells treated with antisense ASOs at 25 μM for 4 days. (n=3). Student's t-test*p<0.05, and error bars indicate ±SD.

FIG. 5. Splice switching ASOs (ASO-52 and ASO-15) and prior art anti-CD73 gapmers (ASO-83 and ASO-84) reduce extracellular 5′ecto-nucleotidase with similar potency (n=2).

FIG. 6. Splice switching ASOs (ASO-52 and ASO-15) do not induce caspase 3 activation. Colo205 cells were treated, and after 4 days caspase 3 activity and cell viability were measured and the ratio of the control (untreated) samples set as 1 (n=3).

FIGS. 7A and 7B. Splice switching ASOs (ASO-15 and ASO-52) show superior target specificity over prior art anti-CD73 gapmers (ASO-83 and ASO-84). FIG. 7A: Volcano plots of differential transcript levels between splice switching ASOs (ASO-15 and ASO-52) and prior art anti-CD73 gapmers (ASO-83 and ASO-84), and non-treated cells as determined by RNA sequencing (n=3). Shaded areas include genes identified as differentially expressed between the corresponding ASO-treated and untreated cells. FIG. 7B: Summary of significant off-target transcript perturbations for each ASO-treated cells compared to untreated cells.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide as described herein is man-made, and is chemically synthesized, and is typically purified or isolated. The oligonucleotide as described herein may comprise one or more modified nucleosides or nucleotides, such as 2′ sugar modified nucleosides.

### Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. In one embodiment, the antisense oligonucleotides are single stranded. It is understood that the single stranded oligonucleotides as described herein can form hairpins or intermolecular duplex structures (duplex between two molecules of the same oligonucleotide), as long as the degree of intra or inter self-complementarity is less than 50% across of the full length of the oligonucleotide.

Advantageously, the single stranded antisense oligonucleotide as described herein does not contain RNA nucleosides, since this will decrease nuclease resistance.

In one embodiment, the antisense oligonucleotide as described herein comprises one or more modified nucleosides or nucleotides, such as 2′ sugar modified nucleosides. Furthermore, it is advantageous that the nucleosides which are not modified are DNA nucleosides.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments, all the nucleotides of the oligonucleotide constitute the contiguous nucleotide sequence. In some embodiments, the oligonucleotide comprises the contiguous nucleotide sequence, and may optionally comprise further nucleotide(s), for example a nucleotide linker region which may be used to attach a functional group to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides

Nucleotides are the building blocks of oligonucleotides and polynucleotides, and for the purposes described herein include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In one embodiment, the modified nucleoside comprises a modified sugar moiety. The term “modified nucleoside” may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotide described herein may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide described herein.

In one embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments, at least 50% of the internucleoside linkages in the oligonucleotide, or contiguous nucleotide sequence thereof, are modified, such as at least 55%, such as at least 60%, such as at least 65%, such as at least 70%, such as at least 75%, such as at least 80%, such as at least 85%, such as at least 90%, or such as at least 95% of the internucleoside linkages in the oligonucleotide, or contiguous nucleotide sequence thereof, are modified. In some embodiments, all of the internucleoside linkages of the oligonucleotide, or contiguous nucleotide sequence thereof, are modified. It will be recognized that, in some embodiments the nucleosides which link the oligonucleotide described herein to a non-nucleotide functional group, such as a conjugate, may be phosphodiester. In some embodiments, all of the internucleoside linkages of the oligonucleotide, or contiguous nucleotide sequence thereof, are nuclease resistant internucleoside linkages.

Modified internucleoside linkages may be selected from the group consisting of phosphorothioate, diphosphorothioate and boranophosphate. In some embodiments, the modified internucleoside linkages are compatible with the Rnase H recruitment of the oligonucleotide described herein, for example phosphorothioate, diphosphorothioate or boranophosphate.

In some embodiments, the internucleoside linkage comprises sulphur (S), such as a phosphorothioate internucleoside linkage.

Phosphorothioate internucleoside linkages are particularly useful due to nuclease resistance, beneficial pharmacokinetics and ease of manufacture. In some embodiments, at least 50% of the internucleoside linkages in the oligonucleotide, or contiguous nucleotide sequence thereof, are phosphorothioate, such as at least 55%, such as at least 60%, such as at least 65%, such as at least 70%, such as at least 75%, such as at least 80%, such as at least 85%, such as at least 90%, or such as at least 95% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some embodiments, the oligonucleotide comprises one or more neutral internucleoside linkages, particularly internucleoside linkages selected from the group consisting of phosphotriester, methylphosphonate, MMI, amide-3, formacetal and thioformacetal.

Further internucleoside linkages are disclosed in WO2009/124238 (incorporated herein by reference). In one embodiment, the internucleoside linkage is selected from linkers disclosed in WO2007/031091 (incorporated herein by reference). In some embodiments, the internucleoside linkage may be selected from the group consisting of —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, 0-PO(OCH₃)-0-, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, NR^{H}H—CO—NR^{H}—O—O—O—, —O—CO-NR^{H}—, —NR^{H}—CO—CH₂—, —O—CH₂—CO-NR^{H}—, —O—CH₂—CH₂—NR^{H}—, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, and —CH₂—NCH₃—O—CH₂—, wherein R^{H} is selected from the group consisting of hydrogen and C1-4-alkyl.

In one embodiment, all the internucleoside linkages of the contiguous nucleotide sequence of the oligonucleotide are phosphorothioate linkages, or all the internucleoside linkages of the oligonucleotide are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleoside linkages, which according to EP 2 742 135 may for example be tolerated in an otherwise DNA phosphorothioate gap region.

### Nucleobase

The term “nucleobase” as used herein includes the purine (e.g. adenine and guanine) and pyrimidine (e.g. uracil, thymine and cytosine) moiety present in nucleosides and nucleotides which form hydrogen bonds in nucleic acid hybridization. As used herein, the term “nucleobase” also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobase selected from the group consisting of isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from the group consisting of A, T, G, C, and 5-methyl cytosine. Optionally, for LNA oligonucleotides, 5-methyl cytosine LNA nucleosides may be used.

### Modified Oligonucleotide

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides with modified nucleosides.

### Complementarity

The term “complementarity” or “complementary” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (expressed in percent) of a contiguous nucleotide sequence in a nucleic acid molecule (e.g. oligonucleotide) which across the contiguous nucleotide sequence are complementary to a reference sequence (e.g. a target sequence or sequence motif). The percentage of complementarity is thus calculated by determining the number of aligned nucleobases that are complementary (from Watson-Crick base pair) between the oligonucleotide sequence and the reference sequence (wherein alignment is performed such that the reference sequence is in 5′-3′ orientation and the oligonucleotide sequence is in 3′-5′ orientation) as follows: 100 times the fraction of X/Y, wherein X is the number of complementary nucleotides between the oligonucleotide sequence and the reference sequence, and wherein Y is the total number of nucleotides in the oligonucleotide.

In such a comparison, a nucleobase/nucleotide which does not align (i.e. does not form a base pair) is termed a mismatch. Insertions and deletions are not allowed in the calculation of complementarity of a contiguous nucleotide sequence. It will be understood that in determining complementarity, chemical modifications of the nucleobases are disregarded as long as the functional capacity of the nucleobase to form Watson-Crick base pairing is retained (e.g. 5′-methyl cytosine is considered identical to a cytosine for the purpose of calculating % identity).

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a target sequence or sequence motif). The percentage of identity is thus calculated by determining the number of aligned nucleobases that are identical (matching) between the oligonucleotide sequence and the reference sequence (wherein alignment is performed such that the orientation of the oligonucleotide sequence and the reference sequence is the same) as follows: 100 times the fraction of X/Y, wherein X is the number of identical nucleotides between the oligonucleotide sequence and the reference sequence, and wherein Y is the total number of nucleotides in the oligonucleotide. Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson-Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38, and Holdgate et al., 2005, Drug Discovered Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides described herein hybridize to a target nucleic acid with estimated ΔG° values below −10 kcal for oligonucleotides that are 10-30 nucleotides in length. In some embodiments, the degree or strength of hybridization is measured by the standard state Gibbs free energy ΔG°. In some embodiments, the oligonucleotides may hybridize to a target nucleic acid with estimated ΔG° values below −10 kcal, such as below −15 kcal, such as below −20 kcal or such as below −25 kcal for oligonucleotides that are 8-30 nucleotides in length. In some embodiments, the oligonucleotides may hybridize to a target nucleic acid with an estimated ΔG° range of −10 to −60 kcal, such as from −12 to −40 kcal, such as from −15 to −30 kcal, such as from −16 to −27 kcal, or such as from −18 to −25 kcal.

### Target

The term “target” as used herein refers to the mammalian, e.g. human, Cluster of Differentiation 73, herein referred to as CD73 or NT5E, and also known in the art as 5′-nucleotidase (5′-NT), ecto-5′-nucleotidase. CD73, GENE ID No 4907, is encoded on human Chromosome 6: 85449584 . . . 85495791 reverse strand (GRCh38.p13, NC_000006.12), the pre-mRNA is exemplified herein as SEQ ID NO: 1. An example of human WT CD73 mRNA is provided herein as SEQ ID NO: 2. An example of human CD73 Δ7 mRNA is provided herein as SEQ ID NO: 4. An example of human WT CD73 Δ7 protein is provided herein as SEQ ID NO: 3. An example of human CD73 Δ7 protein is provided herein as SEQ ID NO: 5. In the context of the present invention a CD73 protein which comprises the amino acid sequence encoded by CD73 exon 7 is referred to as a WT CD73. In the context of the present invention a CD73 protein which lacks part or all of the amino acid sequence encoded by CD73 exon 7 is referred to as a CD73 Δ7.

### Target Nucleic Acid

According to the present invention, the target nucleic acid is a mammalian CD73 pre-mRNA, e.g. the human CD73 pre-mRNA illustrated herein as SEQ ID NO: 1, or a naturally occurring variant thereof. Advantageously, the target nucleic acid comprises a CD73 pre-mRNA exon 7 sequence, such as SEQ ID NO: 1, or a sequence which is at least 98% complementary to SEQ ID NO: 1.

In some embodiments, the target nucleic acid is selected from the group consisting of SEQ ID NO: 6, and any one of 93 to 174 or naturally occurring variants thereof.

Suitably, the target nucleic acid encodes a CD73 polypeptide, in particular a human CD73 polypeptide which comprises CD73 exon 7, such as SEQ ID NO: 3.

The CD73 Δ7 variant is a naturally occurring CD73 allelic variant which results in the deletion of exon 7 in the mRNA (e.g. SEQ ID NO: 4) resulting in a polypeptide with a deletion corresponding to the amino acid sequence encoded by exon 7—referred to herein as a “CD73 Δ7” or “exon 7 deficient CD73” (see for example SEQ ID NO: 5 which represents an exemplary CD73 polypeptide sequence which lacks the amino acids encoded by exon 7).

Suitably, the target nucleic acid encodes a CD73 polypeptide, in particular a human CD73 polypeptide which lacks CD73 exon 7, such as SEQ ID NO: 5.

The contiguous sequence of nucleobases of the antisense oligonucleotide described herein is typically complementary to a region of the CD73 pre-mRNA (SEQ ID NO:1), as measured across the length of the antisense oligonucleotide, optionally with the exception of one or two mismatches, and optionally excluding nucleotide based linker regions which may link the antisense oligonucleotide to an optional functional group such as a conjugate, or other non-complementary terminal nucleotides (e.g. region D′ or D″). In some embodiments, the target nucleic acid may be a RNA or DNA, such as mRNA, such as a mature mRNA or a pre-mRNA. In some embodiments, the target nucleic acid is a RNA or DNA which encodes mammalian CD73 polypeptide, such as human CD73 polypeptide, e.g. the human CD73 mRNA sequence, such as that disclosed as SEQ ID NO: 1. Further information on exemplary target nucleic acids is provided in Table 4.

TABLE 4
Genome and assembly information for CD73 across species.

| | | | | | | Ensembl reference |
|---|---|---|---|---|---|---|
| | | | Genomic coordinates | | | sequence* accession |
| Species | Chr. | Strand | Start | End | Assembly | number for pre-mRNA |
| Human | 6 | + | 85449584 | 85495791 | GRCh38.p12 | ENSG00000135318 |
| Cynomolgus | 4 | − | 88356057 | 88401550 | Macaca_fascicularis_5 | ENSMFAG00000033027 |
| monkey | | | | | | |
| Rat | 8 | + | 95968652 | 96012696 | Rnor_6.0 | ENSRNOG00000011071 |
| Pig | 1 | + | 54400810 | 54448742 | Sscrofa11.1 | ENSSSCG00000004291 |

“+” = forward strand. The genome coordinates provide the pre-mRNA sequence (genomic sequence; SEQ ID NO: 1). The Ensemble reference provides the mRNA sequence (cDNA sequence).
*Ensembl is a genome browser for vertebrate genomes that supports research in comparative genomics, evolution, sequence variation and transcriptional regulation. It is hosmbl.org.

### WT CD73

WT CD73 refers to the wild-type CD73, defined herein as a CD73 protein, which comprises the amino acid sequence encoded by exon 7 of CD73. Accordingly, the WT CD73 protein shall be a protein, which is encoded by a CD73 mRNA which comprises exon 7. In some embodiments, the WT CD73 protein is encoded by a CD73 mRNA which comprises Exons 1 to 9 (as provided in Table 2 above).

### CD73 Δ7

The antisense oligonucleotide as described herein modulates the splicing of the CD73 pre-mRNA resulting in the expression or enhanced expression of a CD73 polypeptide which lacks one or more amino acids in the region encoded by CD73 pre-mRNA exon 7. In some embodiments, the amino acid region encoded by CD73 exon 7 is absent in the CD73 polypeptide produced by the use of the antisense oligonucleotide described herein. CD73 pre-mRNA which lacks a part or all of exon 7 is referred to herein as “CD73 Δ7 mRNA” or “exon 7 deficient CD73 mRNA”. Accordingly, CD73 protein which lacks a part or all of exon 7 is referred to herein as “CD73 Δ7 protein” or “exon 7 deficient CD73 protein”. In some embodiments, the CD73 Δ7 protein is encoded by a CD73 Δ7 mRNA which comprises Exons 1 to 6, 8 and 9 (as provided in Table 2 above).

The change in ratio of different transcript products (e.g. WT CD73 vs. CD73 Δ7) may be measured by comparing mRNA levels, or levels of the corresponding protein products. Anti-CD73 antibodies may be used for assaying the protein levels of WT CD73 and CD73 Δ7, such as monoclonal or polyclonal antibodies specific for either WT CD73 protein or CD73 Δ7 protein.

### Target Sequence

The term “target sequence” as used herein refers to a sequence of nucleotides present in the target nucleic acid which comprises the nucleobase sequence which is complementary to the antisense oligonucleotide described herein. In some embodiments, the target sequence consists of a region on the target nucleic acid with a nucleobase sequence that is complementary to the contiguous nucleotide sequence of the antisense oligonucleotide described herein. This region of the target nucleic acid may interchangeably be referred to as the target nucleotide sequence, target sequence or target region. In some embodiments, the target sequence is longer than the complementary sequence of a single antisense oligonucleotide, and may, for example represent a preferred region of the target nucleic acid which may be targeted by several antisense oligonucleotides described herein.

The target sequence to which the antisense oligonucleotide is complementary or hybridizes to, generally comprises a contiguous nucleobases sequence of at least 10 nucleotides. The contiguous nucleotide sequence is 10 to 50 nucleotides in length, such as 12 to 30, such as 14 to 20, such as 15 to 18 contiguous nucleotides.

In some embodiments, the antisense oligonucleotide, or contiguous nucleotide sequence thereof, is complementary to a target sequence selected from the group consisting of SEQ ID NO: 6, and 93-174.

In some embodiments, the antisense oligonucleotide, or contiguous nucleotide sequence thereof, is complementary to a target sequence selected from the group consisting of SEQ ID NO: 6.

In some embodiments, the antisense oligonucleotide, or contiguous nucleotide sequence thereof, is complementary to a target sequence selected from the group consisting of SEQ ID NO: 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169,170, 171, 172, 173, and 174.

### Target Cell

The term a “target cell” as used herein refers to a cell which is expressing the target nucleic acid. In some embodiments, the target cell may be in vivo or in vitro. In some embodiments, the target cell is a mammalian cell such as a rodent cell, such as a mouse cell or a rat cell, or a primate cell such as a monkey cell or a human cell. In some embodiments, the target cell is a transgenic animal cell which is expressing the human CD73 target nucleic acid. In some embodiments, the target cell is a cancer cell.

For experimental evaluation a target cell may be used which expresses a target nucleic acid which comprises a target sequence. For in vitro evaluation, and for assaying the ability of an antisense oligonucleotide to modulate the splicing of CD73 pre-mRNA, for example, the target cell may be an A549 cell or a Colo205 cell.

In some embodiments, the target cell expresses CD73 mRNA, such as the CD73 pre-mRNA or CD73 mature mRNA. The poly A tail of CD73 mRNA is typically disregarded for antisense oligonucleotide targeting.

Typically, the target cell expresses the CD73 pre-mRNA, which is processed in the cell to the mature CD73 mRNA, resulting in the expression of the CD73 protein (WT CD73). Advantageously, the antisense oligonucleotides of the invention modulate the splicing of the CD73 pre-mRNA to produce a mature CD73 mRNA which lacks CD73 exon 7 (or a portion of exon 7), resulting in the expression of a CD73 Δ7 variant.

In some embodiments, the target nucleic acid is SEQ ID NO: 1, or a naturally occurring variant thereof. In some embodiments, the target nucleic acid is selected from the group consisting of SEQ ID NO: 6, and 93-174, or a naturally occurring variant thereof.

### Naturally Occurring Variant

The term “naturally occurring variant” refers to variants of the CD73 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the antisense oligonucleotide, the antisense oligonucleotide described herein may therefore target the target nucleic acid and naturally occurring variants thereof.

In some embodiments, the naturally occurring variants have at least 95% homology to a mammalian CD73 target nucleic acid such as at least 98% or at least 99% homology to a mammalian CD73 target nucleic acid, such as SEQ ID NO: 1. In some embodiments, the naturally occurring variants have at least 99% homology to the human CD73 target nucleic acid of SEQ ID NO: 1. In some embodiments, the naturally occurring variants are the polymorphisms listed in Table 5.

TABLE 5
Numerous single nucleotide polymorphisms are
known in the CD73 gene, for example those
disclose din the following table (human pre-mRNA
start/reference sequence is SEQ ID NO: 2)

| | Position | Ancestral | | Which | |
|---|---|---|---|---|---|
| | re. | allele | | allele | Minor |
| | human | (in | | is | allele |
| Variant | pre-mRNA | reference | Derived | minor | fre- |
| name | start | sequence) | allele | allele | quency |
| rs11200647 | 18397 | G | A | A | 0.50 |
| rs4391763 | 18270 | C | A | C | 0.49 |
| rs2672591 | 13244 | A | T | A | 0.49 |
| rs28542373 | 49811 | C | G | C | 0.46 |
| rs2268356 | 44286 | C | T | C | 0.46 |

### Modulation of Splicing

Splice modulation can be used to correct cryptic splicing, modulate alternative splicing, restore the open reading frame, and induce protein knockdown. The term “modulation of splicing” as used herein is to be understood as an overall term for an antisense oligonucleotide's ability to modulate the splicing of the CD73 pre-mRNA to result in the elimination of the CD73 exon 7, and provide effective production of CD73 Δ7 (i.e. a CD73 mRNA which does not comprise the CD73 exon 7).

Modulation of splicing may be determined by reference to a control experiment. It is generally understood that the control is an individual or target cell treated with a saline composition or an individual or target cell treated with a non-targeting oligonucleotide (mock). Furthermore, modulation of splicing may be assayed by RNA sequencing (RNA-Seq), which allows for a quantitative assessment of the different splice products of a pre-mRNA.

### Reducing the Expression of WT CD73

In some embodiments, the antisense oligonucleotides of the invention, when present in a target cell, reduce, i.e. decrease expression of WT CD73. The term “reducing the expression” as used herein is to be understood as an overall term for an antisense oligonucleotide's ability to reduce the amount or the activity of WT CD73 in a target cell. In some embodiments, the reduction in amount or activity is at least 10%, at least 20%, at least 30%, at least 40%, at least 50%, at least 60%, at least 70%, at least 80%, or at least 90% compared to that of control cells. Reduction of activity may be measured by determined by measuring the level of WT CD73 mRNA, or by measuring the level or activity of WT CD73 polypeptide in a cell. Reduction of expression may therefore be determined in vitro or in vivo. It will be understood that splice modulation may result in a reduction of expression of WT CD73 in the cell.

Typically, reduction of expression is determined by determining the level of the target nucleic acid present in or the activity of the encoded protein product, following the administration of an effective amount of an antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermined or otherwise known expression level).

### Enhancing Expression of CD73 Δ7 /CD73 Δ7 Enhancer

In some embodiments, the compounds of the invention, when present in a target cell, may enhance the expression of a CD73 Δ7 mRNA or CD73 Δ7 protein, via modulation of the splicing of exon 7 in the CD73 pre-mRNA. Modulation of exon 7 splicing may be determined by measuring the amount of CD73 Δ7 mRNA or CD73 Δ7 protein in cells treated with the antisense oligonucleotides and in untreated control cells. Thus, the amount of CD73 Δ7 mRNA or CD73 Δ7 protein in the target cell shall be enhanced as compared to the amount in a control cell. In some embodiments, the amount of CD73 Δ7 mRNA or CD73 Δ7 protein is at least 10%, at least 20%, at least 30%, at least 40%, at least 50%, at least 60%, at least 70%, at least 80%, or at least 90% enhanced as compared to the amount of control cells. In some embodiments, the amount of CD73 Δ7 mRNA or CD73 Δ7 protein is at least 100% enhanced as compared to the amount of control cells. In some embodiments, the amount of CD73 Δ7 mRNA or CD73 protein is at least 150% enhanced as compared to the amount of control cells. In some embodiments, the amount of CD73 Δ7 mRNA or CD73 Δ7 protein is at least 200% enhanced as compared to the amount of control cells. In some embodiments, the amount of CD73 Δ7 mRNA or CD73 Δ7 protein is at least 250% enhanced as compared to the amount of control cells.

Accordingly, the antisense oligonucleotides of the present invention are CD73 Δ7 enhancers, i.e. antisense oligonucleotides which result in an enhanced expression of CD73 Δ7 mRNA or CD73 Δ7 protein after administration of an effective amount of the antisense oligonucleotide to a target cell.

A CD73 Δ7 enhancer may be identified by measuring the enhanced expression of CD73 mRNA or CD73 Δ7 protein as compared to control cells, or by measuring an enhanced ratio of expression of CD73 Δ7 as compared to WT CD73 at the mRNA or protein level. As illustrated in the examples, per each sample (ASO-treated or untreated) the absolute percentage of CD73 Δ7 mRNA level compared to the total CD73 mRNA level is calculated as follows:

CD73 Δ7/(CD73 Δ7+WT CD73)

wherein
CD73 Δ7 is the level of CD73 Δ7 mRNA
WT CD73 is the level of WT CD73 mRNA

A response>the untreated control level is therefore indicative of an effective CD73 Δ7 enhancer. Advantageously, the CD73 Δ7 enhancer is capable of eliciting a response of >about 1.5, or >about 2, or >about 2.5.

For example, enhancement of the expression of CD73 Δ7 mRNA or protein may be determined in A549 and/or Colo205 (e.g. as illustrated in the Examples). Thus, the cells treated with the antisense oligonucleotides described herein and the untreated control cells may be cultivated as described in the Examples in order to assess whether or not there is an enhancement of the expression of CD73 Δ7 mRNA or CD73 Δ7 protein.

In some embodiments, the compounds of the invention are capable of both i) enhancing the expression of CD73 Δ7 mRNA or CD73 Δ7 protein in the target cell and ii) reducing the expression of WT CD73 Δ7 mRNA and WT CD73 Δ7 protein in a target cell.

### High Affinity Modified Nucleosides

A high affinity modified nucleoside is a modified nucleotide which, when incorporated into the antisense oligonucleotide enhances the affinity of the antisense oligonucleotide for its complementary target, for example as measured by the melting temperature (T^{m}). A high affinity modified nucleoside described herein results in an increase in melting temperature in the range of +0.5 to +12° C., such as in the range of +1.5 to +10° C., and such as in the range of +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The antisense oligonucleotide described herein may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of antisense oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ substituted nucleosides, and numerous 2′ substituted nucleosides have been found to have beneficial properties when incorporated into antisense oligonucleotides. For example, the 2′ modified sugar may provide enhanced binding affinity and/or increased nuclease resistance to the antisense oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and

Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

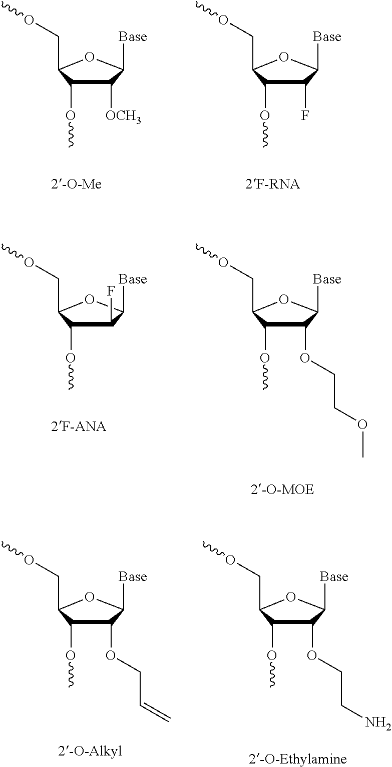

In one embodiment, 2′ substituted sugar modified nucleosides does not include 2′ bridged nucleosides like LNA.

### Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an antisense oligonucleotide for a complementary RNA or DNA molecule. This can be routinely determined by measuring the melting temperature of the antisense oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667. Further non-limiting, exemplary LNA nucleosides are disclosed in Scheme 1.

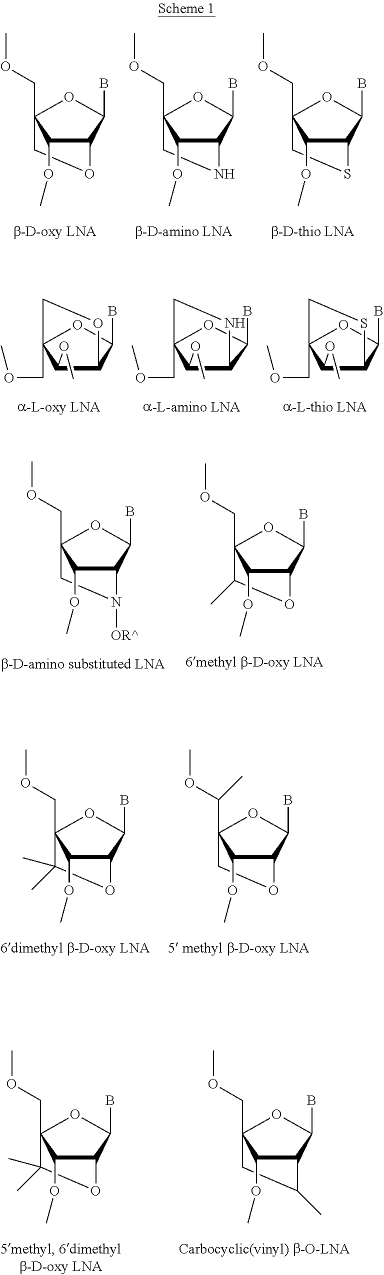

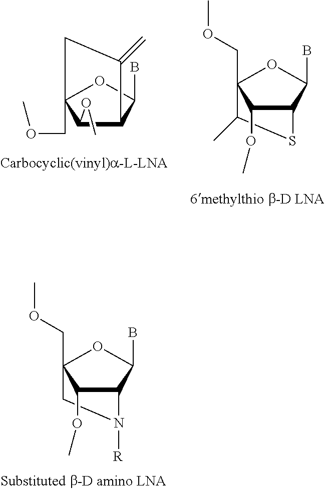

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

The compounds described herein may contain one or more asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

### Pharmaceutically Acceptable Salts

The compounds according to the present invention may exist in the form of their pharmaceutically acceptable salts. The term “pharmaceutically acceptable salt” refers to conventional acid-addition salts or base-addition salts that retain the biological effectiveness and properties of the antisense oligonucleotides described herein.

In a further aspect the invention provides a pharmaceutically acceptable salt of the antisense oligonucleotides, such as a pharmaceutically acceptable sodium salt, ammonium salt or potassium salt.

### Protecting Group

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

### RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an antisense oligonucleotide is deemed capable of recruiting RNase H if it, when provided with a complementary target nucleic acid sequence, has an initial rate, as measured in pmol/l/min, of at least 5%, at least 10%, or more than 20% of the of the initial rate determined when using a antisense oligonucleotide having the same base sequence as the modified antisense oligonucleotide being tested, but containing only DNA monomers with phosphorothioate linkages between all monomers in the antisense oligonucleotide, and using the methodology provided by Example 91-95 of WO01/23613 (hereby incorporated by reference). DNA antisense oligonucleotides are known to effectively recruit RNase H, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNase H degradation of the target. Therefore, the splice modulating antisense oligonucleotides of the invention are preferably not gapmer oligonucleotides. RNase H recruitment may be avoided by limiting the number of contiguous DNA nucleotides in the antisense oligonucleotide—therefore for effective splice modulation mixmers and totalmers designs may therefore be used.

### Totalmers

The term “totalmer” as used herein is a single stranded oligomer, or contiguous nucleotide sequence thereof, which does not comprise DNA or RNA nucleosides, and as such only comprises nucleoside analogue nucleosides. The oligomer, or contiguous nucleotide sequence thereof, maybe a totalmer—indeed various totalmer designs are highly effective as therapeutic oligomers, particularly when targeting microRNA (antimiRs) or as splice switching oligomers (SSOs).

In some embodiments, the totalmer comprises or consists of at least one XYX or YXY sequence motif, such as a repeated sequence XYX or YXY, wherein X is LNA and Y is an alternative (i.e. non-LNA) nucleotide analogue, such as a 2′-OMe RNA unit and 2′-fluoro DNA unit. The above sequence motif may, in some embodiments, be for example XXY, XYX, YXY or YYX.

In some embodiments, the totalmer may comprise or consist of a contiguous nucleotide sequence of 8 to 20 nucleotides in length, such as 8, 9, 10, 11, 12, 13, 14, 15, 16, 17, 18, 19 or 20 nucleotides, such as 12 to 18 nucleotides in length.

In some embodiments, the contiguous nucleotide sequence of the totalmer comprises at least 30%, such as at least 40%, such as at least 50%, such as at least 60%, such as at least 70%, such as at least 80%, such as at least 90%, such as 95%, such as 100% LNA units. The remaining units may be selected from the non-LNA nucleotide analogues referred to herein in, such as those selected from the group consisting of 2′-0 alkyl-RNA unit, 2′-OMe-RNA unit, 2′-amino-DNA unit, 2′-fluoro-DNA unit, LNA unit, PNA unit, HNA unit, INA unit, and a 2′MOE RNA unit, or the group of 2′-OMe RNA unit and 2′-fluoro DNA unit.

In some embodiments, the totalmer consist or comprises of a contiguous nucleotide sequence which consists only of LNA units.

### Mixmers

The term “mixmer” as used herein refers to oligomers which comprise both DNA nucleosides and sugar modified nucleosides, wherein there are insufficient length of contiguous DNA nucleosides to recruit RNase H. Suitable mixmers may comprise up to 1, up to 2, up to 3 or up to 4 contiguous DNA nucleosides. In some embodiments, the mixmers, or contiguous nucleotide sequence thereof, comprise alternating regions of sugar modified nucleosides, and

DNA nucleosides. By alternating regions of sugar modified nucleosides which form a RNA like (3′ endo) conformation when incorporated into the antisense oligonucleotide, with short regions of DNA nucleosides, non-RNase H recruiting antisense oligonucleotides may be made. Advantageously, the sugar modified nucleosides are affinity enhancing sugar modified nucleosides.

Mixmers are often used to provide occupation based modulation of target genes, such as splice modulators or microRNA inhibitors.

In some embodiments, the sugar modified nucleosides in the mixmer, or contiguous nucleotide sequence thereof, comprise or are all LNA nucleosides, such as (S)cET or beta-D-oxy LNA nucleosides.

In some embodiments, all of the sugar modified nucleosides of a mixmer comprise the same sugar modification, for example they may all be LNA nucleosides, or may all be 2′O-MOE nucleosides. In some embodiments, the sugar modified nucleosides of a mixmer may be independently selected from LNA nucleosides and 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the oligonucleotide comprises both LNA nucleosides and 2′ substituted nucleosides, such as 2′ substituted nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the antisense oligonucleotide comprises LNA nucleosides and 2′-O-MOE nucleosides. In some embodiments, the antisense oligonucleotide comprises (S)cET LNA nucleosides and 2′-O-MOE nucleosides. In some embodiments, the mixmer, or contiguous nucleotide sequence thereof, comprises only LNA and DNA nucleosides, such LNA mixmer oligonucleotides which may for example be 8-24 nucleosides in length (see for example, WO2007112754, which discloses LNA antmiR inhibitors of microRNAs).

Various mixmer compounds are highly effective as therapeutic oligomers, particularly when targeting microRNA (antimiRs) or as splice switching oligomers (SSOs). In some embodiments, the mixmer comprises a motif
. . . [L]m[D]n[L]m[D]n[L]m . . . or
. . . [L]m[D]n[L]m[D]n[L]m[D]n[L]m . . . or
. . . [L]m[D]n[L]m[D]n[L]m[D]n[L]m[D]n[L]m . . . or
. . . [L]m[D]n[L]m[D]n[L]m[D]n[L]m[D]n[L]m[D]n[L]m . . . or
. . . [L]m[D]n[L]m[D]n[L]m[D]n[L]m[D]n[L]m[D]n[L]m[D]n[L]m . . . or
. . . [L]m[D]n[L]m[D]n[L]m[D]n[L]m[D]n[L]m[D]n[L]m[D]n[L]m[D]n[L]m . . . or
. . . [L]m[D]n[L]m[D]n[L]m[D]n[L]m[D]n[L]m[D]n[L]m[D]n[L]m[D]n[L]m[D]n[L]m . . . or
. . . [L]m[D]n[L]m[D]n[L]m[D]n[L]m[D]n[L]m[D]n[L]m[D]n[L]m[D]n[L]m[D]n[L]m[D]n[L]m,
   wherein L represents sugar modified nucleoside such as a LNA or 2′ substituted nucleoside (e.g. 2′-O-M0E), D represents DNA nucleoside, and wherein each m is independently selected from 1-6, and each n is independently selected from 1, 2, 3 and 4, such as 1-3. In some embodiments, each L is a LNA nucleoside. In some embodiments, at least one L is a LNA nucleoside and at least one L is a 2′-O-MOE nucleoside. In some embodiments, each L is independently selected from LNA and 2′-O-MOE nucleoside.

In some embodiments, the mixmer may comprise or consist of a contiguous nucleotide sequence of between 10 and 24 nucleotides, such as 11, 12, 13, 14, 15, 16, 17, 18, 19, 20, 21, 22 or 23 nucleotides. In some embodiments, the mixmer may comprise or consist of a contiguous nucleotide sequence of between 12 and 22 nucleotides, such as 12, 13, 14, 15, 16, 17, 18, 19, 20, 21, or 22 nucleotides. In some embodiments, the mixmer may comprise or consist of a contiguous nucleotide sequence of between 14 and 20 nucleotides, such as 14, 15, 16, 17, 18, 19, or 20 nucleotides. In some embodiments, the mixmer may comprise or consist of a contiguous nucleotide sequence of between 16 and 18 nucleotides, such as 16, 17, or 18 nucleotides. In some embodiments, the mixmer may comprise or consist of a contiguous nucleotide sequence of 18 nucleotides.

In some embodiments, the contiguous nucleotide sequence of the mixmer comprises at least 30%, such as at least 40%, such as at least 50% LNA units.

In some embodiments, the mixmer comprises or consists of a contiguous nucleotide sequence of repeating pattern of nucleotide analogues and naturally occurring nucleotides, or one type of nucleotide analogue and a second type of nucleotide analogue. The repeating pattern, may, for instance be: every second or every third nucleotide is a nucleotide analogue, such as LNA, and the remaining nucleotides are naturally occurring nucleotides, such as DNA, or are a 2′ substituted nucleotide analogue such as 2′MOE of 2′fluoro analogues as referred to herein, or, in some embodiments selected form the groups of nucleotide analogues referred to herein. It is recognized that the repeating pattern of nucleotide analogues, such as LNA units, may be combined with nucleotide analogues at fixed positions—e.g. at the 5′ or 3′ termini. In some embodiments, the first nucleotide of the oligomer, counting from the 3′ end, is a nucleotide analogue, such as a LNA nucleotide or a 2′-O-MOE nucleoside. In some embodiments, which maybe the same or different, the second nucleotide of the oligomer, counting from the 3′ end, is a nucleotide analogue, such as a LNA nucleotide or a 2′-O-MOE nucleoside.

In some embodiments, which maybe the same or different, the 5′ terminal of the oligomer is a nucleotide analogue, such as a LNA nucleotide or a 2′-O-MOE nucleoside. In some embodiments, the mixmer comprises at least a region comprising at least two consecutive nucleotide analogue units, such as at least two consecutive LNA units. In some embodiments, the mixmer comprises at least a region comprising at least three consecutive nucleotide analogue units, such as at least three consecutive LNA units.

### Conjugate

The term “conjugate” as used herein refers to an antisense oligonucleotide which is covalently linked to a non-nucleotide moiety (conjugate moiety or region C or third region). The conjugate moiety may be covalently linked to the antisense oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the antisense oligonucleotide directly or through a linking moiety (e.g. linker or tether). Linkers serve to covalently connect a third region, e.g. a conjugate moiety (region C), to a first region, e.g. an antisense oligonucleotide or contiguous nucleotide sequence complementary to the target nucleic acid (region A).

In some embodiments, the conjugate or oligonucleotide conjugate described herein may optionally, comprise a linker region (second region or region B and/or region Y) which is positioned between the antisense oligonucleotide or contiguous nucleotide sequence complementary to the target nucleic acid (region A or first region) and the conjugate moiety (region C or third region).

In one embodiment, region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment, the biocleavable linker is susceptible to S1 nuclease cleavage. In one embodiment, the nuclease susceptible linker comprises 1-10 nucleosides, such as 1, 2, 3, 4, 5, 6, 7, 8, 9 or 10 nucleosides. In one embodiment, the nuclease susceptible linker comprises 2 -6 nucleosides. In one embodiment, the nuclease susceptible linker comprises 2-4 linked nucleosides comprising at least two consecutive phosphodiester linkages, such as at least 3 or 4 or 5 consecutive phosphodiester linkages. In one embodiment, the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an antisense oligonucleotide (region A or first region). The region Y linkers may comprise a chain structure or an oligomer of repeating units such as ethylene glycol, amino acid units or amino alkyl groups. The antisense oligonucleotide conjugates described herein can be constructed of the following regional elements A-C, A-B-C, A-B-Y-C, A-Y-B-C or A-Y-C. In some embodiments, the linker (region Y) is an amino alkyl, such as a C2-C36 amino alkyl group, including, for example C6 to C12 amino alkyl groups. In one embodiment, the linker (region Y) is a C6 amino alkyl group.

### Treatment

The term “treatment” as used herein refers to both, treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

### Antisense Oligonucleotides of the Invention

The invention relates to antisense oligonucleotides capable of inducing CD73 splice switching, such as enhancing the expression of CD73 Δ7. The spice switching activity is achieved by hybridizing the antisense oligonucleotide to a target nucleic acid encoding a CD73 polypeptide.

In one embodiment, the target nucleic acid may be a mammalian CD73 sequence, such as SEQ ID NO: 1. In one embodiment, the target nucleic acid comprises the target sequence of SEQ ID NO: 6.

In one embodiment, the antisense oligonucleotide targets CD73 pre-mRNA (such as SEQ ID NO: 1) and thereby induces splice switching of the CD73 pre-mRNA. In one embodiment, the antisense oligonucleotide targets CD73 pre-mRNA thereby producing exon 7 deficient CD73 mRNA (CD73 Δ7, such as SEQ ID NO: 4) resulting in the expression of a CD73 polypeptide which lacks part or all of the CD73 exon 7 (such as SEQ ID NO: 5). In one embodiment, the antisense oligonucleotide targets CD73 pre-mRNA thereby reducing WT CD73 mRNA (such as SEQ ID NO: 2), resulting in reduced expression of a WT CD73 polypeptide (such as SEQ ID NO: 3). In one embodiment, the antisense oligonucleotide targets an intronic region or exonic region (such as depicted in Tables 2 and 3). In one embodiment, the antisense oligonucleotide targets a splice site, such as the 3′ splice site of Exon 7. In one embodiment, the antisense oligonucleotide targets a splice site, such as the 5′ splice site of Exon 7. In one embodiment, the antisense oligonucleotide targets the target region of SEQ ID NO: 6.

In some embodiments, the antisense oligonucleotide described herein induces conversion of CD73 pre-mRNA into exon 7 deficient CD73 mRNA (CD73 Δ7). In one embodiment, the antisense oligonucleotide induces conversion into CD73 Δ7 by at least 50%, at least 60%, at least 70%, at least 80%, at least 90% or 100% compared to the normal level of conversion into CD73 Δ7.

In some embodiments, the antisense oligonucleotide described herein is capable of inducing conversion of CD73 pre-mRNA into CD73 Δ7 by at least 60% in vitro following application of 5 μM antisense oligonucleotide to A549 cells. In some embodiments, the antisense oligonucleotide described herein is capable of inducing conversion of CD73 pre-mRNA into CD73 Δ7 by at least 80% in vitro following application of 25 μM antisense oligonucleotide to Colo205 cells.

Suitably, the examples provide assays, which may be used to measure CD73 spice switching activity (e.g. Examples 2 and 3). The splice switching is triggered by the hybridization between a contiguous nucleotide sequence of the antisense oligonucleotide and the target nucleic acid. In some embodiments, the antisense oligonucleotide described herein comprises mismatches between the antisense oligonucleotide and the target nucleic acid. Despite mismatches hybridization to the target nucleic acid may still be sufficient to show a desired conversion into CD73 Δ7. Reduced binding affinity resulting from mismatches may be compensated by increased number of nucleotides in the antisense oligonucleotide and/or an increased number of modified nucleosides capable of increasing the binding affinity to the target, such as 2′ sugar modified nucleosides, including LNA, present within the antisense oligonucleotide sequence.

An aspect of the present invention relates to an antisense oligonucleotide targeting a mammalian CD73 pre-mRNA, wherein the antisense oligonucleotide modulates splicing of the mammalian CD73 pre-mRNA, and comprises a contiguous nucleotide sequence of at least 10 nucleotides in length with at least 90% complementarity, such as 100% complementarity to the mammalian CD73 pre-mRNA.

In some embodiments, the antisense oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary to a region of the target nucleic acid or a target sequence. In one embodiment, the target sequence is SEQ ID NO: 1. In one embodiment, the target sequence is SEQ ID NO: 6. In one embodiment, the target sequence is selected from the group consisting of any one of SEQ ID NO: 93-174.

In some embodiments, the antisense oligonucleotide of the invention, or contiguous nucleotide sequence thereof is fully complementary (100% complementary) to a region of the target nucleic acid, or in some embodiments may comprise one or two mismatches between the antisense oligonucleotide and the target nucleic acid.

In some embodiments, the antisense oligonucleotide comprises a contiguous nucleotide sequence of 10 to 30 nucleotides in length with at least 90% complementary, such as fully (or 100%) complementary, to a target nucleic acid region present in SEQ ID NO: 1. In some embodiments, the antisense oligonucleotide sequence is 100% complementary to a corresponding target nucleic acid region present in SEQ ID NO: 1. In some embodiments the antisense oligonucleotide sequence is 100% complementary to a corresponding target nucleic acid region present in SEQ ID NO: 6. In some embodiments the antisense oligonucleotide sequence is 100% complementary to a corresponding target nucleic acid region selected from the group consisting of SEQ ID NO: 93-174.

In some embodiments, the antisense oligonucleotide or contiguous nucleotide sequence thereof is capable of enhancing the expression of exon 7 deficient mammalian CD73 mRNA (SEQ ID NO: 4). In some embodiments, the antisense oligonucleotide or contiguous nucleotide sequence thereof is capable of reducing the expression of WT mammalian CD73 mRNA (SEQ ID NO: 2). In some embodiments, the antisense oligonucleotide or contiguous nucleotide sequence thereof is capable of enhancing the expression of exon 7 deficient mammalian CD73 mRNA (SEQ ID NO: 4) and reducing the expression of WT mammalian CD73 mRNA (SEQ ID NO: 2).

In some embodiments, the antisense oligonucleotide of the invention comprises or consists of 10 to 35 nucleotides in length, such as from 12 to 30, such as 15 to 25, such as from 16 to 20, or such as 17 to 19 contiguous nucleotides in length. In a preferred embodiment, the antisense oligonucleotide comprises or consists of 18 nucleotides in length.

In some embodiments, the antisense oligonucleotide or contiguous nucleotide sequence thereof comprises or consists of 25 or less nucleotides, such as 22 or less nucleotides, such as 20 or less nucleotides, such as 19, 18, 17 or 16 nucleotides. It is to be understood that any range given herein includes the range endpoints. Accordingly, if an antisense oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21, 22, 23, 24, 25, 26, 27, 28, 29 or 30 contiguous nucleotides in length. In a preferred embodiment, the antisense oligonucleotide comprises or consists of 18 nucleotides in length.

In some embodiments, the antisense oligonucleotide or contiguous nucleotide sequence comprises or consists of a sequence selected from the group consisting of sequences listed in Table 8 (Materials and Method section).

In some embodiments, the antisense oligonucleotide or contiguous nucleotide sequence comprises or consists of 10 to 30 nucleotides in length with at least 90% identity, preferably 100% identity, to the sequence of SEQ ID NO: 6 In some embodiments, the antisense oligonucleotide or contiguous nucleotide sequence comprises or consists of 10 to 30 nucleotides in length with at least 90% identity, preferably 100% identity, to a sequence selected from the group consisting of SEQ ID NO: 93 to 174 (see RNA Target Sequences listed in table 8). In some embodiments, the antisense oligonucleotide or contiguous nucleotide sequence comprises or consists of 10 to 30 nucleotides in length with at least 90% identity, preferably 100% identity, to the sequence of SEQ ID NO: 107 or 144 (see RNA Target Sequences listed in table 8).

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the antisense oligonucleotide sequence is generally termed antisense oligonucleotide design.

The antisense oligonucleotides of the invention are designed with modified nucleosides and DNA nucleosides. Advantageously, high affinity modified nucleosides are used.

In an embodiment, the antisense oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the antisense oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the antisense oligonucleotide comprises one or more sugar modified nucleosides, such as 2′ sugar modified nucleosides. Preferably the antisense oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment, the antisense oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are phosphorothioate or boranophosphate internucleoside linkages. In some embodiments, all the internucleotide linkages in the contiguous sequence of the antisense oligonucleotide are phosphorothioate linkages.

In some embodiments, the antisense oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the antisense oligonucleotide are LNA nucleosides, such as 80%, such as 85%, such as 90% of the modified nucleosides are LNA nucleosides. In a still further embodiment, all the modified nucleosides in the antisense oligonucleotide are LNA nucleosides. In a further embodiment, the antisense oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the antisense oligonucleotide or contiguous nucleotide sequence to have at least 1 LNA nucleoside at the 5′ end and at least 2 LNA nucleosides at the 3′ end of the nucleotide sequence. In some embodiments, the antisense oligonucleotide has a sequence and design as depicted in Table 8 (column Oligonucleotide Compound (Design)). In some embodiments, the antisense oligonucleotide has the sequence and design of GtAAttGTgcCTgaGgGT. In some embodiments, the antisense oligonucleotide has the sequence and design of TggCcGtAgcGgtGcaCG. In some embodiments, the capital letter designates a beta-D-oxy LNA nucleoside. In some embodiments, the lower case letter designates a DNA nucleoside. In some embodiments, all internucleoside linkages are phosphorothioate internucleoside linkages. In some embodiments, all LNA cytosine units are 5-methyl-cytosine.

For some embodiments of the invention, the antisense oligonucleotide comprises or consist of a sequence selected from the group consisting of SEQ ID NO: 7-88.

For certain embodiments of the invention, the antisense oligonucleotide comprises or consists of SEQ ID NO: 21 or 58.

For some embodiments of the invention, the antisense oligonucleotide is selected from the group of antisense oligonucleotide compounds with ASO ID NO: ASO-1 to ASO-82.

For certain embodiments of the invention, the antisense oligonucleotide is ASO ID NO: ASO-15 or ASO-52.

In a further embodiment of the invention, the antisense oligonucleotide may comprise at least one stereodefined internucleoside linkages, such as a stereodefined phosphorothioate internucleoside linkage.

A key advantage of generating stereodefined antisense oligonucleotide variants is the ability to increase the diversity across a sequence motif, and select stereo defined antisense oligonucleotides including sub-libraries of stereo defined antisense oligonucleotides, which have improved medicinal chemical properties as compared to a parent antisense oligonucleotide.

For in vivo or in vitro applications, the antisense oligonucleotide described herein is typically capable of modulating the splicing of exon 7 of the human CD73 pre-mRNA, resulting in the expression or enhanced expression of CD73 Δ7 polypeptide.

### Method of Manufacture

In a further aspect, the invention provides methods for manufacturing the antisense oligonucleotide described herein comprising reacting nucleotide units and thereby forming covalently linked contiguous nucleotide units comprised in the antisense oligonucleotide. Preferably, the method uses phophoramidite chemistry (see for example Caruthers et al.). In a further embodiment, the method further comprises reacting the contiguous nucleotide sequence with a conjugating moiety (ligand) to covalently attach the conjugate moiety to the antisense oligonucleotide. In a further aspect, a method is provided for manufacturing the composition of the invention, comprising mixing the antisense oligonucleotide or conjugated oligonucleotide of the invention with a pharmaceutically acceptable diluent, solvent, carrier, salt and/or adjuvant.

### Pharmaceutical Salt

The antisense oligonucleotides according to the present invention may exist in the form of their pharmaceutically acceptable salts. The term “pharmaceutically acceptable salt” refers to conventional acid-addition salts or base-addition salts that retain the biological effectiveness and properties of the compounds of the present invention.

In a further aspect, the invention provides a pharmaceutically acceptable salt of the nucleic acid molecules or a conjugate thereof, such as a pharmaceutically acceptable sodium salt, ammonium salt or potassium salt.

### Pharmaceutical Composition

In a further aspect, pharmaceutical compositions are provided comprising any of the aforementioned antisense oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the pharmaceutically acceptable diluent is sterile phosphate buffered saline. In some embodiments, the antisense oligonucleotide is used in the pharmaceutically acceptable diluent at a concentration of 50-300 μM solution.

Suitable formulations for use as described herein are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Antisense oligonucleotides or oligonucleotide conjugates as described herein may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the antisense oligonucleotide or oligonucleotide conjugate described herein is a prodrug. In one embodiment, the conjugate moiety is cleaved off the antisense oligonucleotide once the prodrug is delivered to the site of action, e.g. the target cell.

### Applications

The antisense oligonucleotides described herein may be utilized as research reagents for, for example, diagnostics, therapeutics and prophylaxis.

In research, such antisense oligonucleotides may be used to specifically modulate the synthesis of CD73 polypeptide in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the polypeptide, thereby prevent polypeptide formation or by degrading or inhibiting a modulator of the gene or mRNA producing the polypeptide.

If employing the antisense oligonucleotide described herein in research or diagnostics the target nucleic acid may be a cDNA or a synthetic nucleic acid derived from DNA or RNA.

Described herein is an in vivo or in vitro method for modulating the splicing of a CD73 pre-mRNA in a target cell expressing CD73, said method comprising administering an antisense oligonucleotide described herein in an effective amount to said cell.

In some embodiments, the target cell is a mammalian cell. In one embodiment, the target cell is a human cell. The target cell may be an in vitro cell culture or an in vivo cell forming part of a tissue in a mammal. In one embodiment, the target cell is present in a tumor. In one embodiment, the target cell is a tumor cell.

In diagnostics, the antisense oligonucleotides may be used to detect and quantitate expression of CD73 splice variants, such as CD73 Δ7 and/or WT CD73, in cells and tissues by northern blotting, in-situ hybridization or similar techniques.

For therapeutics, the antisense oligonucleotides may be administered to an animal or a human, suspected of having a disease or disorder, which can be treated by modulating the splicing of CD73 pre-mRNA.

Provided herein are methods for treating or preventing a disease, comprising administering a therapeutically or prophylactically effective amount of an antisense oligonucleotide, oligonucleotide conjugate, or pharmaceutically acceptable salt thereof, or a pharmaceutical composition to a subject suffering from or susceptible to the disease such as diseases or disorders selected from the group consisting of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer and renal-cell cancer.

Provided herein is an antisense oligonucleotide, oligonucleotide conjugate, or pharmaceutically acceptable salt thereof, or a pharmaceutical composition as defined herein for use as a medicament.

The antisense oligonucleotide, oligonucleotide conjugate, or pharmaceutically acceptable salt thereof, or the pharmaceutical composition described herein is typically administered in an effective amount.

Further provided herein is the use of the antisense oligonucleotide or oligonucleotide conjugate for the manufacture of a medicament for the treatment of a disorder as referred to herein, or for a method of the treatment of as a disorder as referred to herein.

The disease or disorder, as referred to herein, is associated with expression of CD73 variants, such as CD73 Δ7 and WT CD73. In some embodiments, the disease or disorder may be associated with a mutation in the CD73 gene or a gene whose protein product is associated with or interacts with CD73 variants. Therefore, in some embodiments, the target nucleic acid is a mutated form of the CD73 sequence and in other embodiments, the target nucleic acid is a regulator of the CD73 sequence.

The methods described herein are preferably employed for treatment or prophylaxis against diseases caused by abnormal levels and/or activity of CD73 variants, such as CD73 Δ7 and WT CD73.

Further provided herein is the use of an antisense oligonucleotide, oligonucleotide conjugate or pharmaceutically acceptable salt thereof, or a pharmaceutical composition for the manufacture of a medicament for the treatment of abnormal levels and/or activity of CD73 variants, such as CD73 Δ7 and WT CD73 .

Further provided herein are antisense oligonucleotides, oligonucleotide conjugates or pharmaceutically acceptable salts thereof, or pharmaceutical compositions for use in the treatment of diseases or disorders selected from colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer and renal-cell cancer.

### Administration

In some embodiments, the antisense oligonucleotide, oligonucleotide conjugate or the pharmaceutically acceptable salt thereof, or the pharmaceutical composition of the present invention may, for example be administered by a parenteral route including intravenous, intraarterial, subcutaneous, intraperitoneal or intramuscular injection or infusion. In some embodiments, the antisense oligonucleotide, oligonucleotide conjugate or pharmaceutical composition is administered intravenously. In another embodiment, the antisense oligonucleotide, oligonucleotide conjugate or pharmaceutical composition is administered subcutaneously.

In some embodiments, the antisense oligonucleotide, oligonucleotide conjugate or the pharmaceutically acceptable salt thereof, or the pharmaceutical composition is administered orally or rectally. In some embodiments, the antisense oligonucleotide, oligonucleotide conjugate or pharmaceutical composition is administered orally or rectally for the treatment of colorectal cancer, non-small cell lung cancer, small-cell lung cancer, merkel-cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer and renal-cell cancer.

In some embodiments, the antisense oligonucleotide, oligonucleotide conjugate or the pharmaceutically acceptable salt thereof, or the pharmaceutical composition of the invention is administered at a dose of 0.1-15 mg/kg, such as from 0.2-10 mg/kg, such as from 0.25-5 mg/kg. The administration can be once a week, every second week, every third week or even once a month.

The invention also provides for the use of the antisense oligonucleotide, oligonucleotide conjugate, or the pharmaceutically acceptable salt thereof, or the pharmaceutical composition of the invention as described for the manufacture of a medicament wherein the medicament is in a dosage form for subcutaneous administration.

### Combination Therapies

In some embodiments, the antisense oligonucleotide, oligonucleotide conjugate or the pharmaceutically acceptable salt thereof, or the pharmaceutical composition described herein is for use in a combination treatment with another therapeutic agent. The therapeutic agent can for example be the standard of care for the diseases or disorders described above. In one embodiment, the therapeutic agent is a check-point inhibitor. In one embodiment, the therapeutic agent targets molecules selected from the group consisting of CTLA4, PD-1, and PD-L1. In one embodiment, the therapeutic agent is selected from the group consisting of ipilimumab, nivolumab, pembrolizumab, atezolizumab, avelumab, durvalumab, and cemiplimab.

### EXAMPLES

### Example 1—Materials and Methods

Antisense oligonucleotides ASO-1 to ASO-84 were synthesized and their ability to modulate the splicing of the CD73 pre-mRNA to enhance the ratio of CD73 Δ7 mRNA as compared to WT CD73 mRNA was evaluated. Specifically, it was evaluated whether the antisense oligonucleotides enhance the expression of CD73 Δ7 mRNA and/or and reduce the expression of WT CD73 mRNA in treated versus untreated cells.

### Compound and Data Table

TABLE 8
The motif sequences of nucleobases in the antisense oligonucleotides of the invention
are shown as SEQ ID NO: 7-88.

| | | | | | | | exS | | exS |
|---|---|---|---|---|---|---|---|---|---|
| | | | | | | exS | activity | exS | activity |
| | | | | | | activity | in | activity | in |
| | SEQ | | | SEQ | | in A549 | Colo20 | in A549 | Colo205 |
| | ID | | Oligonucleotide | ID | | (%) | 5 (%) | (%) | (%) |
| ASO # | NO: | Motif Sequence | Compound (Design) | NO: | RNA Target Sequence | 25 μM | 25 μM | 5 μM | 5 μM |
| ASO-1 | 7 | aatagcttgagaaaatgc | AaTaGCtTGaGAaAAtGC | 93 | gcattttctcaagctatt | 22.10 | 15.29 | 26.36 | 16.05 |
| ASO-2 | 8 | gaaaatagcttgagaaaa | GAaaATaGCtTGaGAaAA | 94 | ttttctcaagctattttc | 26.10 | 13.20 | 26.41 | 13.43 |
| ASO-3 | 9 | aaggaaaatagcttgaga | AaGGaAaATaGCtTGaGA | 95 | tctcaagctattttcctt | 20.60 | 20.84 | 25.22 | 17.23 |
| ASO-4 | 10 | aagaaggaaaatagcttg | AaGAaGGaAaATaGCtTG | 96 | caagctattttccttctt | 29.79 | 12.85 | 22.48 | 12.78 |
| ASO-5 | 11 | ggcaagaaggaaaatagc | GgCAaGAaGGaAaATaGC | 97 | gctattttccttcttgcc | 36.07 | 27.97 | 30.09 | 23.19 |
| ASO-6 | 12 | tgaggcaagaaggaaaat | TgAGgCAaGAaGGaAaAT | 98 | attttccttcttgcctca | 27.10 | 19.29 | 29.33 | 13.30 |
| ASO-7 | 13 | agatgaggcaagaaggaa | AgATgAGgCaAGaAGgAA | 99 | ttccttcttgcctcatct | 25.92 | 25.11 | 25.98 | 20.97 |
| ASO-8 | 14 | cacagatgaggcaagaag | CAcAgATgAGgCAaGaAG | 100 | cttcttgcctcatctgtg | 40.25 | 35.51 | 30.38 | 25.69 |
| ASO-9 | 15 | agtcacagatgaggcaag | AGtcACagATgaGGcaAG | 101 | cttgcctcatctgtgact | 33.66 | 31.16 | 36.16 | 18.78 |
| ASO-10 | 16 | ggtagtcacagatgaggc | GgtAGtcACagATgAgGC | 102 | gcctcatctgtgactacc | 49.39 | 53.52 | 43.14 | 40.51 |
| ASO-11 | 17 | gagggtagtcacagatga | GAggGTaGtcAcaGatGA | 103 | tcatctgtgactaccctc | 31.22 | 36.57 | 33.16 | 33.33 |
| ASO-12 | 18 | cctgagggtagtcacaga | CcTgaGgGTaGtcAcaGA | 104 | tctgtgactaccctcagg | 59.68 | 57.77 | 48.48 | 41.49 |
| ASO-13 | 19 | gtgcctgagggtagtcac | GtGccTgaGgGtaGtcAC | 105 | gtgactaccctcaggcac | 55.73 | 44.21 | 45.80 | 30.67 |
| ASO-14 | 20 | attgtgcctgagggtagt | ATtGtgCcTgaGgGtaGT | 106 | actaccctcaggcacaat | 47.43 | 48.37 | 40.92 | 35.78 |
| ASO-15 | 21 | gtaattgtgcctgagggt | GtAAttGTgcCTgaGgGT | 107 | accctcaggcacaattac | 55.16 | 69.70 | 48.52 | 52.50 |
| ASO-16 | 22 | caggtaattgtgcctgag | CagGTaATtgTgcCTgAG | 108 | ctcaggcacaattacctg | 29.81 | 23.41 | 27.29 | 13.71 |
| ASO-17 | 23 | tcccaggtaattgtgcct | TccCaGgtAAttGTgcCT | 109 | aggcacaattacctggga | 32.43 | 23.14 | 33.29 | 15.82 |
| ASO-18 | 24 | ttctcccaggtaattgtg | TTcTccCagGTaATtgTG | 110 | cacaattacctgggagaa | 55.65 | 68.62 | 40.41 | 42.00 |
| ASO-19 | 25 | aggttctcccaggtaatt | AgGTtcTccCagGTaaTT | 111 | aattacctgggagaacct | 56.11 | 50.57 | 44.87 | 36.39 |
| ASO-20 | 26 | gccaggttctcccaggta | GccAggTTctCccAggTA | 112 | tacctgggagaacctggc | 37.31 | 47.77 | 30.08 | 49.80 |
| ASO-21 | 27 | gcagccaggttctcccag | GcaGccAggTTctCccAG | 113 | ctgggagaacctggctgc | 38.46 | 67.90 | 37.86 | 67.56 |
| ASO-22 | 28 | acagcagccaggttctcc | AcaGcaGccAggTTctCC | 114 | ggagaacctggctgctgt | 41.12 | 36.45 | 34.39 | 24.77 |
| ASO-23 | 29 | aatacagcagccaggttc | AAtACaGcaGccAGgtTC | 115 | gaacctggctgctgtatt | 41.48 | 48.33 | 35.83 | 28.24 |
| ASO-24 | 30 | ggcaatacagcagccagg | GgcAatAcaGcaGCcaGG | 116 | cctggctgctgtattgcc | 31.72 | 41.98 | 31.04 | 22.06 |
| ASO-25 | 31 | aagggcaatacagcagcc | AAggGcaATacAGcAgCC | 117 | ggctgctgtattgccctt | 39.65 | 37.11 | 26.16 | 17.67 |
| ASO-26 | 32 | ccaaagggcaatacagca | CCaaAggGcaATaCAgCA | 118 | tgctgtattgccctttgg | 44.35 | 37.40 | 31.32 | 25.01 |
| ASO-27 | 33 | cctccaaagggcaataca | CcTccAAaGgGCaATaCA | 119 | tgtattgccctttggagg | 31.04 | 40.89 | 24.31 | 43.50 |
| ASO-28 | 34 | gtgcctccaaagggcaat | GtGcCTcCaAagGGcaAT | 120 | attgccctttggaggcac | 15.35 | 14.30 | 17.57 | 8.39 |
| ASO-29 | 35 | aatgtgcctccaaagggc | AaTgTgcCTccAaaGgGC | 121 | gccctttggaggcacatt | 28.79 | 26.18 | 21.96 | 13.20 |
| ASO-30 | 36 | tcaaatgtgcctccaaag | TcAaATgTgCCtCCaaAG | 122 | ctttggaggcacatttga | 33.33 | 26.16 | 23.32 | 15.87 |
| ASO-31 | 37 | aggtcaaatgtgcctcca | AgGtcAAatGTgcCTcCA | 123 | tggaggcacatttgacct | 60.00 | 21.46 | 28.97 | 14.70 |
| ASO-32 | 38 | actaggtcaaatgtgcct | AcTAgGTcAaATgTgcCT | 124 | aggcacatttgacctagt | 26.63 | 20.85 | 29.75 | 14.83 |
| ASO-33 | 39 | tggactaggtcaaatgtg | TgGAcTaGgtCAaATgTG | 125 | cacatttgacctagtcca | 35.58 | 46.99 | 25.06 | 22.83 |
| ASO-34 | 40 | aactggactaggtcaaat | AaCTgGAcTaGGtCAaAT | 126 | atttgacctagtccagtt | 20.25 | 15.34 | 23.36 | 13.18 |
| ASO-35 | 41 | tttaactggactaggtca | TTtAacTgGAcTaGGtCA | 127 | tgacctagtccagttaaa | 29.10 | 41.01 | 28.16 | 26.92 |
| ASO-36 | 42 | ccttttaactggactagg | CcTTttAaCTgGAcTaGG | 128 | cctagtccagttaaaagg | 29.47 | 49.42 | 27.44 | 25.98 |
| ASO-37 | 43 | gaaccttttaactggact | GAacCTttTAaCTgGaCT | 129 | agtccagttaaaaggttc | 33.51 | 19.49 | 28.53 | 15.53 |
| ASO-38 | 44 | gtggaaccttttaactgg | GTgGAacCTttTaACtGG | 130 | ccagttaaaaggttccac | 54.90 | 71.67 | 51.26 | 61.56 |
| ASO-39 | 45 | agggtggaaccttttaac | AGgGTgGAacCTtTtaAC | 131 | gttaaaaggttccaccct | 21.74 | 27.98 | 22.65 | 18.87 |
| ASO-40 | 46 | ttcagggtggaacctttt | TTcaGgGTggAAcCTtTT | 132 | aaaaggttccaccctgaa | 25.31 | 35.24 | 27.65 | 29.65 |
| ASO-41 | 47 | ttcttcagggtggaacct | TtcTTcaGgGTggAAcCT | 133 | aggttccaccctgaagaa | 45.75 | 66.21 | 47.94 | 53.58 |
| ASO-42 | 48 | gccttcttcagggtggaa | GccTtcTtCAggGTggAA | 134 | ttccaccctgaagaaggc | 41.12 | 48.49 | 36.54 | 38.39 |
| ASO-43 | 49 | aaggccttcttcagggtg | AagGccTtcTtcAGggTG | 135 | caccctg aag aaggcctt | 31.00 | 27.66 | 21.19 | 15.37 |
| ASO-44 | 50 | tcaaaggccttcttcagg | TcAaaGGccTTcTTcaGG | 136 | cctgaagaaggcctttga | 21.72 | 49.87 | 23.87 | 30.82 |
| ASO-45 | 51 | tgctcaaaggccttcttc | TgcTcAAaGGccTTctTC | 137 | gaagaaggcctttgagca | 17.86 | 35.35 | 22.49 | 22.18 |
| ASO-46 | 52 | ctatgctcaaaggccttc | CtATgCTcAaaGgCctTC | 138 | gaaggcctttgagcatag | 23.10 | 29.58 | 24.12 | 20.58 |
| ASO-47 | 53 | acgctatgctcaaaggcc | AcGCtaTgcTcAaaGgCC | 139 | ggcctttgagcatagcgt | 31.66 | 39.04 | 31.96 | 23.32 |
| ASO-48 | 54 | tgcacgctatgctcaaag | TgCAcgCTaTgCTcAaAG | 140 | ctttgagcatagcgtgca | 26.36 | 31.35 | 25.88 | 18.78 |
| ASO-49 | 55 | cggtgcacgctatgctca | CgGtGcAcgCTatGctCA | 141 | tgagcatagcgtgcaccg | 36.28 | 73.40 | 39.74 | 54.04 |
| ASO-50 | 56 | tagcggtgcacgctatgc | TaGcGgtGcACgcTatGC | 142 | gcatagcgtgcaccgcta | 24.18 | 21.71 | 22.49 | 14.64 |
| ASO-51 | 57 | ccgtagcggtgcacgcta | CcGtaGcgGtGcaCgcTA | 143 | tagcgtgcaccgctacgg | 28.90 | 43.63 | 22.34 | 29.45 |
| ASO-52 | 58 | tggccgtagcggtgcacg | TggCcGtAgcGgtGcaCG | 144 | cgtgcaccgctacggcca | 39.11 | 83.50 | 31.76 | 61.15 |
| ASO-53 | 59 | gactggccgtagcggtgc | GacTgGccGtaGcGgtGC | 145 | gcaccgctacggccagtc | 44.24 | 70.83 | 39.12 | 51.01 |
| ASO-54 | 60 | gtggactggccgtagcgg | GtgGacTgGccGtAgcGG | 146 | ccgctacggccagtccac | 43.03 | 70.44 | 30.37 | 47.49 |
| ASO-55 | 61 | ccagtggactggccgtag | CcaGtgGacTgGccGtAG | 147 | ctacggccagtccactgg | 21.03 | 26.08 | 21.25 | 16.29 |
| ASO-56 | 62 | tctccagtggactggccg | TcTccAgtGgActGgcCG | 148 | cggccagtccactggaga | 28.28 | 77.19 | 25.12 | 52.88 |
| ASO-57 | 63 | aactctccagtggactgg | AacTcTccAGtgGActGG | 149 | ccagtccactggagagtt | 28.54 | 22.70 | 24.96 | 14.81 |
| ASO-58 | 64 | aggaactctccagtggac | AGgAacTctCcaGTggAC | 150 | gtccactggagagttcct | 41.60 | 69.81 | 30.83 | 53.56 |
| ASO-59 | 65 | tgcaggaactctccagtg | TgcAggAActCTccAgTG | 151 | cactggagagttcctgca | 50.33 | 65.69 | 42.40 | 54.46 |
| ASO-60 | 66 | acctgcaggaactctcca | AccTgcAGgAacTcTcCA | 152 | tggagagttcctgcaggt | 18.22 | 25.77 | 19.23 | 27.02 |
| ASO-61 | 67 | cccacctgcaggaactct | CccAcCtGcaGgaActCT | 153 | agagttcctgcaggtggg | 27.46 | 13.66 | 18.53 | 12.37 |
| ASO-62 | 68 | ccgcccacctgcaggaac | CcGccCacCtGcaGgaAC | 154 | gttcctgcaggtgggcgg | 19.51 | 27.40 | 14.73 | 29.74 |
| ASO-63 | 69 | ttaccgcccacctgcagg | TtAccGccCacCtGcaGG | 155 | cctgcaggtgggcggtaa | 20.86 | 13.77 | 19.72 | 15.58 |
| ASO-64 | 70 | gacttaccgcccacctgc | GacTtAccGccCacCtGC | 156 | gcaggtgggcggtaagtc | 47.30 | 65.17 | 32.80 | 46.72 |
| ASO-65 | 71 | ggtgacttaccgcccacc | GgtGacTtAccGccCaCC | 157 | ggtgggcggtaagtcacc | 21.26 | 19.57 | 26.32 | 12.28 |
| ASO-66 | 72 | atgggtgacttaccgccc | AtGgGtgAcTtAccGcCC | 158 | gggcggtaagtcacccat | 25.53 | 13.66 | 23.12 | 9.07 |
| ASO-67 | 73 | aggatgggtgacttaccg | AggATggGTgaCTtAcCG | 159 | cggtaagtcacccatcct | 26.76 | 6.31 | 26.21 | 10.27 |
| ASO-68 | 74 | tacaggatgggtgactta | TacAGgATggGTgActTA | 160 | taagtcacccatcctgta | 19.23 | 10.76 | 27.23 | 7.03 |
| ASO-69 | 75 | ccctacaggatgggtgac | CccTacAggATgGgTgAC | 161 | gtcacccatcctgtaggg | 19.81 | 7.16 | 17.20 | 8.91 |
| ASO-70 | 76 | cagccctacaggatgggt | CagCccTacAggATggGT | 162 | acccatcctgtagggctg | 24.43 | 7.76 | 14.78 | 5.94 |
| ASO-71 | 77 | ggccagccctacaggatg | GgcCaGcCctAcaGgaTG | 163 | catcctgtagggctggcc | 19.34 | 4.15 | 19.02 | 5.68 |
| ASO-72 | 78 | atgggccagccctacagg | ATggGccAgcCctAcaGG | 164 | cctgtagggctggcccat | 11.80 | 5.59 | 17.98 | 6.02 |
| ASO-73 | 79 | tggatgggccagccctac | TggATggGccAgcCctAC | 165 | gtagggctggcccatcca | 22.06 | 3.99 | 21.60 | 6.61 |
| ASO-74 | 80 | ctttggatgggccagccc | CTttGgaTggGccAgcCC | 166 | gggctggcccatccaaag | 19.57 | 5.13 | 20.05 | 5.70 |
| ASO-75 | 81 | tcactttggatgggccag | TcaCTttGgATggGccAG | 167 | ctggcccatccaaagtga | 7.91 | 4.61 | 11.52 | 5.54 |
| ASO-76 | 82 | atgtcactttggatgggc | ATgtCacTTtGgATggGC | 168 | gcccatccaaagtgacat | 8.51 | 4.46 | 7.16 | 4.20 |
| ASO-77 | 83 | gccatgtcactttggatg | GccATgtCAcTTtGgaTG | 169 | catccaaagtgacatggc | 42.92 | 38.17 | 30.62 | 29.48 |
| ASO-78 | 84 | aatgccatgtcactttgg | AAtgCCatGTcaCTttGG | 170 | ccaaagtgacatggcatt | 17.61 | 10.02 | 24.82 | 6.97 |
| ASO-79 | 85 | ccaggtaattgtgcctgagg | CcaGgtAatTgtGccTgaGG | 171 | cctcaggcacaattacctgg | 29.20 | 16.38 | 29.71 | 33.52 |
| ASO-80 | 86 | cccaggtaattgtgcctgag | CccAgGtaAttGtGccTgAG | 172 | ctcaggcacaattacctggg | 25.70 | 19.28 | 25.54 | 12.67 |
| ASO-81 | 87 | tcccaggtaattgtgcctga | TccCagGtaAttGtgCctGA | 173 | tcaggcacaattacctggga | 19.38 | 11.17 | 26.34 | 8.76 |
| ASO-82 | 88 | ctcccaggtaattgtgcctg | CtcCcaGgtAatTgtGccTG | 174 | caggcacaattacctgggag | 21.58 | 23.08 | 33.29 | 11.61 |
| ASO-83 | 89 | gatttcccagtgccat | GATttcccagtgcCAT | 175 | atggcactgggaaatc | NA | NA | NA | NA |
| ASO-84 | 90 | gcactcgacacttggt | GCActcgacacttGGT | 176 | accaagtgtcgagtgc | NA | NA | NA | NA |
| Scr Ctrl 1 | 91 | gtacatccacttattcaa | GTAcATcCACttATtCAA | 177 | ttgaataagtggatgtac | 20.42 | 9.43 | 20.42 | 9.43 |
| Scr Ctrl 2 | 92 | ccaaatcttataataactac | CcAAAtcttataataACtAC | 178 | gtagttattataagatttgg | | | | |
| Vehicle | NA | NA | NA | | | 23.19 | 10.05 | 23.19 | 10.05 |

The corresponding RNA Target Sequences to which the contiguous sequence of nucleobases present in the antisense oligonucleotide (i.e. motif sequences) are fully (and reverse) complementary to are shown as SEQ ID NO: 93-174. The compounds are designated as an ASO ID No. (#), with the antisense oligonucleotide structure (5′-3′), wherein a capital letter designates a beta-D-oxy LNA nucleoside, a lower case letter designates a DNA nucleoside, and all internucleoside linkages are phosphorothioate internucleoside linkages. The splice switching activity of the antisense oligonucleotides in A549 and Colo205 cells at a final concentration of 25 μM and 5 μM is given, respectively.

### Cell Culture and ASO Treatment

A549 cells were cultured in F12/K medium supplemented with 10% FBS and 25 pg/m1 Gentamicin. Colo205 cells were cultured in RPMI medium supplemented with 10% FBS and 1× pen/strep. A549 or Colo205 cells were seeded at 3,000 cells/well in 96 well-plate format. After 12 hours, cells were treated with antisense oligonucleotide (ASO) compounds at final concentrations of 5 μM or 25 μM for 4 days by unassisted uptake. As negative controls, PBS-treated and scramble ASO-treated cells were included in the experiments.

### Splice Switching Activity Measurement

Cells were washed with PBS and total RNA was extracted using RNeasy® Mini kit (Qiagen, 74181) according to the manufacturer's instructions. cDNA was prepared with iScript™ Advanced cDNA synthesis kit (#1725037, BioRad) following the manufacturer's instructions. ddPCR triplex assay was used to quantitate: unskipped NT5E mRNA (exon 6+exon 7+exon 8), skipped NT5E mRNA (exon 6+exon 8, Δex7), and HPRT1 mRNA (Table 9). Basal expression level of Δex7 was determined for PBS-treated samples as the ratio between unskipped NT5E mRNA and skipped NT5E. This was subsequently used to calculate the absolute percentage (%) of splice switching activity for ASO-treated samples. Primers and probes used in the ddPCR are shown in Table 9 below.

TABLE 9
List of primers and probes used for
ddPCR analysis.

| | | SEQ |
|---|---|---|
| Oligo # | Sequence | ID # |
| NT5E_1 | ggaggtggtatccggtc | 179 |
| NT5E_2 | ccgaaaacctggagacag | 180 |
| NT5E_ex7s_ | aacgcaacaatggaatccatgtggtg/56-FAM/ | 181 |
| probe | aacgcaaca/Zen/atggaatccatgtggtg/ | |
| | 3IABkFQ | |
| NT5E_wt_ | aacgcaacaatggcacaattacct/5HEX/ | 182 |
| probe | aacgcaaca/Zen/atggcacaattacct/ | |
| | 3IABkFQ | |
| HPRT1_1 | gcgatgtcaataggactccag | 183 |
| HPRT1_2 | ttgttgtaggatatgcccttga | 184 |
| HPRT1_ | agcctaagatgagagttcaagttgagtttgg/ | 185 |
| probe1 | 5HEX/agcctaaga/ZEN/tgagagttcaagt | |
| | tgagtttgg/3IABkFQ/ | |
| HPRT1_ | agcctaagatgagagttcaagttgagtttgg/ | 186 |
| probe2 | 56-FAM/agcctaaga/ZEN/tgagagttcaa | |
| | gttgagtttgg/3IABkFQ/ | |

### Ecto-5′-Nucleotidase Activity Measurement

After 4 days of ASO-treatment at a final concentration of 25 μM, Colo205 cells were washed twice in enzymatic activity buffer (20 mM HEPES, 2 mM MgCl₂ , 120 mM NaCl, 5 mM KCl, 10 mM glucose, pH 7.4). Fresh enzymatic activity buffer was supplemented with adenosine monophosphate (AMP, final concentration 500 μM) and added to the cells. After 1-hour incubation (37° C., 5% CO₂), supernatant was collected and processed for free phosphate quantification using Malachite Green assay (#MAK307-1 KT, Sigma) following the manufacturer's instructions.

### Immunoblot Analysis

Colo205 cells were treated for 4 days with ASOs at a final concentration of 25 μM. Cells were washed in PBS and lysed in RIPA buffer (#89900, ThermoFisher Scientific) supplemented with phosphatase/protease inhibitors cocktail (#78442, ThermoFisher Scientific). CD73 and GAPDH proteins were resolved and detected using Wes (Simple Wes) on a 2-230 kDa separation matrix. Densitometry analysis was used to quantitate protein signal intensity. Rabbit anti-CD73 antibody (D7F9A, Cell Signaling) and mouse anti-GAPDH antibody (ab11035, Abcam) were used.

### Cell Viability and Caspase 3/7 Measurements

Colo205 cells were seeded at 3,000 cells/well in 96 well-plate format. After 12 hours, cells were treated with ASO compounds at increasing concentrations for 4 days by unassisted uptake. Cell viability and caspase 3 activity were measured using Cell Titer Glo® (#G7571, Promega) and Caspase Glo® 3/7 assay kits (#G8090, Promega), following the manufacturer's instructions. The ratio of caspase activity and cell titer measurements for the untreated samples was used as normalizer (=1).

### RNA Sequencing and Differential Gene Expression Analysis

Colo205 cells were treated for 4 days with the splice switching ASO-15 and ASO-52, and the prior art anti-CD73 gapmers ASO-83 and ASO-84 (10 μM) or left untreated (n=3). Total RNA was extracted using the RNeasy® Plus Mini kit from QIAGEN. Stranded mRNA libraries were prepared using KAPA mRNA HyperPrep Kit for Illumina following the manufacturer's instructions, and sequenced on an Illumina NextSeq500. About 15 million reads were demultiplexed per sample. Sequenced reads were QC-tested, trimmed and mapped using CLC Genomic workbench. Significant differentially expressed genes were defined as having a −log₁₀(p-value)>2 and a log₂ fold change>1.

### Example 2—Determination of Splice Switching Activity

### Screening of Antisense Oligonucleotides for Splice Switching Activity

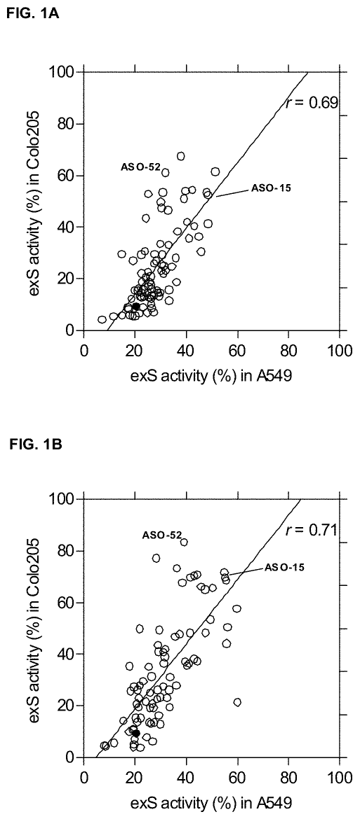

82 LNA mixmers were designed, synthesized and screened for splice switching activity of human CD73 in A549 and Colo205 cell lines as described above. A list of antisense oligonucleotides with their sequences and corresponding exon skipping activity (exS) is depicted in Table 8 above. ASOs targeting either the 3′ splice site or exonic regions have been identified to induce up to 60% (in A549) and 80% (in Colo205) conversion of the full length CD73 mRNA into the Δex7 isoform. Splice switching activity of the tested compounds shows a correlation of r=0.69 (at 5 μM) and r=0.71 (at 25 μM) between A549 and Colo205 cells, suggesting that in two different biological systems the antisense oligonucleotides produce exon skipping by a similar mode of action (FIGS. 1A and 1B).

### Evaluation of Splice Switching Activity of ASO-52 and ASO-15

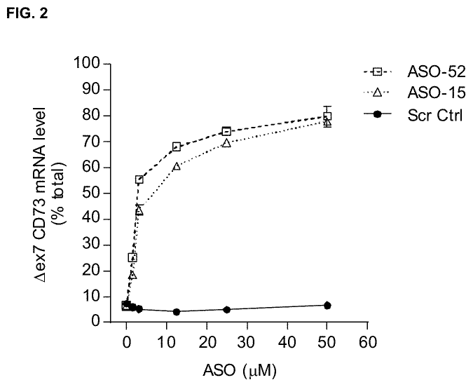

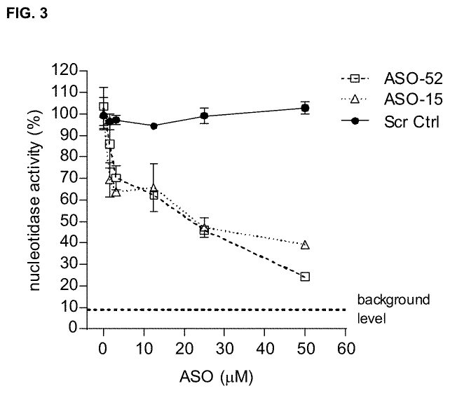

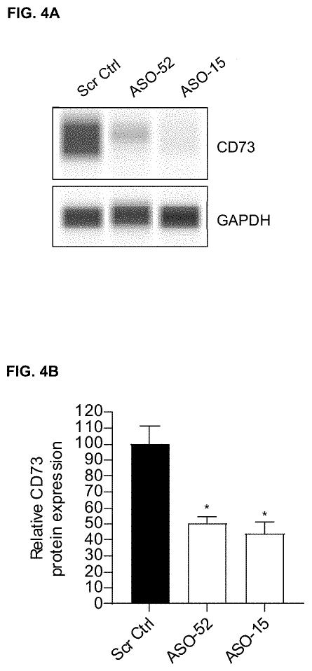

The molecular effects of the most potent antisense oligonucleotides (ASO-52 and ASO-15) were further evaluated at the mRNA level for exon skipping activity, and at the protein level for enzymatic inhibition of ecto-5′-nucleotidaseactivity. Treatment of Colo205 cells with either ASO-52 or ASO-15 induced a dose-dependent conversion of the full length CD73 mRNA into the Δex7 isoform, while a scramble ASO did not produce such effect (FIG. 2). Similarly, Colo205 cells were treated for 4 days with either a scramble ASO, ASO-52 or ASO-15, and extracellular 5′ecto-nucleotidase activity was measured as the production of free phosphate from the hydrolysis of externally supplemented AMP. ASO-52 and ASO-15 inhibited CD73 activity in a dose-dependent fashion (FIG. 3). In order to characterize the effect of ASO-52 and ASO-15 on target protein level, CD73 total protein abundance was measured by immunoblot analysis. Treatment with ASO-52 and ASO-15 led to a significant reduction of the overall CD73 protein content, confirming that the induction of Δex7 results in the destabilization of the total CD73 protein (FIGS. 4A and 4B).

### Example 3—Comparison with Prior Art Gapmers (ASO-83 and ASO-84)

### Comparison of Splice Switching Activity

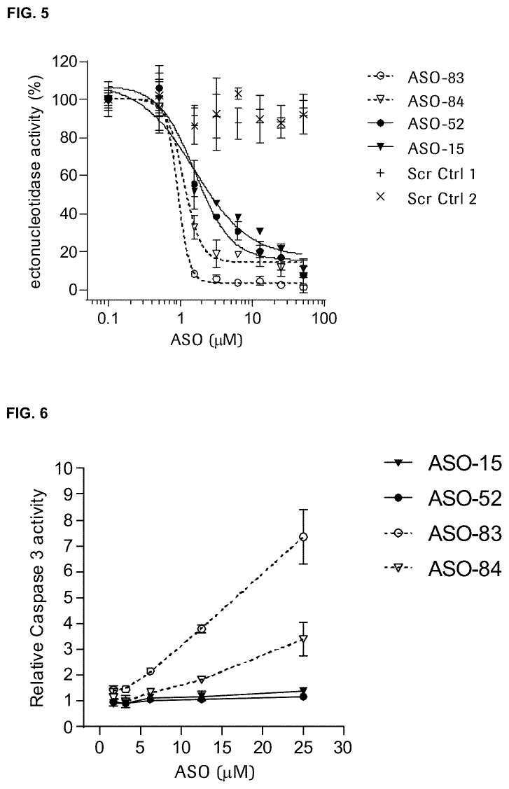

In order to further characterize the splice switching ASO-52 and ASO-15, two among the most potent anti-CD73 gapmers described by Secarna (WO2018/065627) were synthesized (ASO-83 and ASO-84). Since gapmers and splice switching ASOs exploit different molecular mechanisms to achieve the inhibition of CD73, their potency was evaluated by IC50 determination on extracellular 5′ectonucleoidase activity. Colo205 cells were treated at different concentration of ASOs for 4 days. Both prior art anti-CD73 gapmers (ASO-83 and ASO-84) and the splice switching ASOs (ASO-52 and ASO-15) showed similar IC50 values for CD73 inhibition, demonstrating that splice switching ASOs inducing CD73 exon 7 skipping are an effective alternative therapeutic modality for the reduction of CD73 activity. Results are presented in FIG. 5 and Table 10.

TABLE 10
IC50 values for splice switching ASO-15 and ASO-52,
and prior art anti-CD73 gapmers ASO-83 and ASO-84

| Oligo # | IC50 (μM) | Std. Error (±) |
|---|---|---|
| ASO-83 | 0.92 | 0.14 |
| ASO-84 | 1.11 | 0.15 |
| ASO-52 | 1.60 | 0.29 |
| ASO-15 | 1.58 | 0.43 |

### Comparison of Cytotoxic Effect

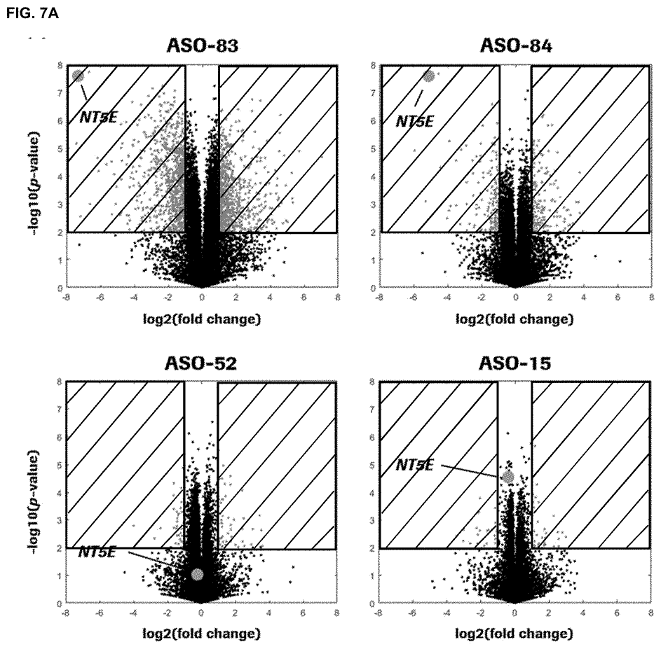

Moreover, we evaluated the cytotoxic effect of the splice switching ASO-15 and ASO-52, with the prior art anti-CD73 gapmers ASO-82 and ASO-83. Colo205 were treated for 4 days at increasing concentration of ASOs. Caspase 3 activation was measured and normalized for cell viability (to account for proliferation and cell number effect, FIG. 6). Splice switching ASO-15 and ASO-52 did not show significant induction of caspase 3 activity at any concentration evaluated (max=25 μM). Conversely, both prior art gapmers (ASO-83 and ASO-84) caused a marked increase of apoptosis induction in a dose-dependent fashion. These data indicate that splice switching ASOs possess an advantageous in vitro safety profile compared to the prior art anti-CD73 gapmers.

### Comparison of Differential Gene Expression

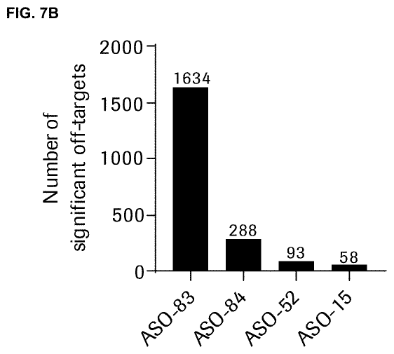

In order to characterize the global perturbation of gene expression caused by the treatment with the splice switching ASO-15 and ASO-52, and the prior art anti-CD73 gapmers ASO-83 and ASO-84, we performed RNA-seq and differential gene expression analysis. Colo205 cells were treated with different ASOs for 4 days at 10μM, or left untreated (n=3). Differential expression analysis was performed comparing each ASO-treated samples with untreated ones. The magnitude of expression change and the statistical significance level per each gene are represented as volcano plots. Treatment with ASO-15 and ASO-52 is associated with a reduced number of off-target genes compared to ASO-83 and ASO-84 treatment. The absolute number of differentially expressed genes is also indicated (FIGS. 7A and 7B).

### REFERENCES

de Andrade Mello P, Coutinho-Silva R, and Savio L E B. Multifaceted Effects of Extracellular Adenosine Triphosphate and Adenosine in the Tumor-Host Interaction and Therapeutic Perspectives. Front. Immunol. 8, 1526 (2017)

Caruthers M H, Barone A D, Beaucage S L, Dodds D R, Fisher E F, McBride L J, Matteucci M, Stabinsky Z, Tang J Y. Chemical Synthesis of Deoxyaligonucleotides by the Phosphoramidite, Method Methods in Enzymology vol. 154, pages 287-313 (1987)

Fredholm B B, Lizerman, A P, Jacobson K A, Klotz K N, and Linden J. Nomenclature and classification of adenosine receptors. Pharmacol. Rev. 53, 527-552 (2001)

Havens M A and Hastings M L. Splice-switching Antisense Oligonucieotides as Therapeutic Drugs. Nucleic Acids Res. Aug 19; 44(14): 6549-6563 (2016)

Leclerc BG, Charlebois R, Chouinard G, Allard B, Pommey S, Saad F, and Stagg J. CD73 Expression Is an Independent Prognostic Factor in Prostate Cancer. Clin Cancer Res. 22(1):158-66 (2016)

Loi S, Pommey S, Haibe-Kains B, Beavis P A, Darcy P K, Smyth M J, and Stagg J. CD73 promotes anthracycline resistance and poor prognosis in triple negative breast cancer. Proc Natl Acad Sci USA 110, 11091-11096 (2013)

Resta R, Hooker S W, Hansen K R, Laurent A B, Park J L, Blackburn M R, Knudsen T B, and Thompson L F. Murine ecto-5′-nucleotidase (CD73): cDNA cloning and tissue distribution. Gene 133, 171-177 (1993)

Silva-Vilches C, Ring S, and Mahnke K. ATP and Its Metabolite Adenosine as Regulators of Dendritic Cell Activity. Front. Immunol. 9, 2581 (2018)

Snider N T, Altshuler P J, Wan S, Welling T H, Cavalcoli J, and Omary M B. Alternative splicing of human NTSE in cirrhosis and hepatocellular carcinoma produces a negative regulator of ecto-5′-nucleotidase (CD73). Mol Biol Cell. 25(25):4024-33 (2014)

1. An antisense oligonucleotide targeting a mammalian CD73 pre-mRNA, wherein the antisense oligonucleotide modulates splicing of the mammalian CD73 pre-mRNA and comprises a contiguous nucleotide sequence of at least 10 nucleotides in length with at least 90% complementarity to the mammalian CD73 pre-mRNA.

2. The antisense oligonucleotide of claim 1, wherein the antisense oligonucleotide or contiguous nucleotide sequence thereof is capable of enhancing the expression of exon 7 deficient mammalian CD73 mRNA.

3. The antisense oligonucleotide of claim 1, wherein the antisense oligonucleotide or contiguous nucleotide sequence thereof is capable of reducing the expression of WT mammalian CD73 mRNA.

4. The antisense oligonucleotide of claim 1, wherein the antisense oligonucleotide or contiguous nucleotide sequence thereof is capable of enhancing the expression of exon 7 deficient mammalian CD73 mRNA and reducing the expression of WT mammalian CD73 mRNA.

5. The antisense oligonucleotide of claim 1, wherein the antisense oligonucleotide or contiguous nucleotide sequence thereof is 10-40 nucleotides in length.

6. The antisense oligonucleotide of claim 1, wherein the antisense oligonucleotide or contiguous nucleotide sequence thereof is complementary to SEQ ID NO: 1 or SEQ ID NO: 6.

7. (canceled)

8. The antisense oligonucleotide of claim 1, wherein the antisense oligonucleotide or contiguous nucleotide sequence thereof is complementary to a sequence selected from the group consisting of SEQ ID NO: 93-174.

9. The antisense oligonucleotide of claim 1, wherein the antisense oligonucleotide comprises an antisense oligonucleotide mixmer or totalmer.

10. The antisense oligonucleotide of claim 5, wherein the antisense oligonucleotide or contiguous nucleotide sequence thereof is 10-20 nucleotides in length.

11. The antisense oligonucleotide of claim 1, wherein the contiguous nucleotide sequence of the antisense oligonucleotide comprises a sequence selected from any one of SEQ ID NO: 7-88.

12. The antisense oligonucleotide of claim 1, wherein the antisense oligonucleotide comprises an antisense oligonucleotide selected from the group consisting of any one of ASO ID NO: ASO-1-ASO-82.

13. A conjugate comprising the antisense oligonucleotide of claim 1, and at least one conjugate moiety covalently attached to said antisense oligonucleotide.

14. A pharmaceutically acceptable salt of the antisense oligonucleotide of claim 1.

15. A pharmaceutical composition comprising the antisense oligonucleotide of claim 1, and a pharmaceutically acceptable diluent, solvent, carrier, salt and/or adjuvant.

16. An in vivo or in vitro method for modulating the splicing of a mammalian CD73 pre-mRNA in a target cell which is expressing mammalian CD73, said method comprising administering the antisense oligonucleotide of claim 1, in an effective amount to said cell.

17. The method of claim 16, wherein the administration of the antisense oligonucleotide results in enhanced expression of exon 7 deficient mammalian CD73 mRNA.

18. The method of claim 16, wherein the administration of the antisense oligonucleotide results in reduced expression of WT mammalian CD73 mRNA.

19. The method of claim 16, wherein the administration of the antisense oligonucleotide results in enhanced expression of exon 7 deficient mammalian CD73 mRNA and reduced expression of WT mammalian CD73 mRNA.

20-21. (canceled)

22. The method of claim 16, wherein the mammalian CD73 mRNA is a human CD73 mRNA.

23. A method for treating or preventing cancer comprising administering a therapeutically or prophylactically effective amount of the antisense oligonucleotide of claim 1, to a subject suffering from or susceptible to cancer.

24-26. (canceled)

27. The antisense oligonucleotide of claim 1, wherein the antisense oligonucleotide comprises a contiguous nucleotide sequence of at least 10 nucleotides in length with 100% complementarity to the mammalian CD73 pre-mRNA.

28. The method of claim 23, wherein the cancer is selected from the group consisting of colorectal cancer, non-small cell lung cancer, small-cell lung cancer, Merkel cell cancer, mesothelioma cancer, endometrial cancer, ovarian cancer, uveal cancer, adrenocortical cancer, germ-cell cancer, esophagogastric cancer, cutaneous squamous-cell cancer, anal cancer, melanoma cancer, pancreatic cancer, breast cancer, head and neck squamous carcinoma, sarcoma, hepatocellular carcinoma, prostate cancer, cervical cancer, glioblastoma cancer, urothelial cancer, and renal-cell cancer.
